(19) United States
(12) Patent Application Publication
(10) Pub. No.: US 2023/0134760 A1
(43) Pub. Date: May 4, 2023

(54) CAR AND NRF2 DUAL ACTIVATOR AGENTS FOR CYCLOPHOSPHAMIDE-BASED AND DOXORUBICIN-BASED TREATMENTS OF CANCER

(71) Applicants: Hongbing Wang, Ellicott City, MD (US); Fengtian Xue, Potomac, MD (US); Linhao LI, Ellicott City, MD (US)

(72) Inventors: Hongbing Wang, Ellicott City, MD (US); Fengtian Xue, Potomac, MD (US); Linhao LI, Ellicott City, MD (US)

(21) Appl. No.: 17/909,334

(22) PCT Filed: Mar. 4, 2021
(86) PCT No.: PCT/US2021/020900

§ 371 Date: Sep. 2, 2022

Related U.S. Application Data
(60) Provisional application No. 62/984,994, filed on Mar. 4, 2020.

(51) Int. Cl. A61K 31/429; C07D 498/04; C07D 513/04; A61K 31/675; A61K 31/704; A61K 31/475; A61K 31/573; A61K 31/424; A61P 35/00

(52) U.S. Cl. CPC A61K 31/429; C07D 498/04; C07D 513/04; A61K 31/675; A61K 31/704; A61K 31/475; A61K 31/573; A61K 31/424; A61P 35/00

(57) ABSTRACT

The disclosure relates to selective small molecule dual activators of human constitutive androstane receptor (hCAR) and nuclear factor erythroid 2-related factor 2 (Nrf2), pharmaceutical compositions thereof, and use thereof for the treatment of hematologic malignancies and other cancers. The small molecule dual hCAR and Nrf2 activators in combination with CPA based chemotherapy regimen provides a synergistic effect to help promote cytoxicity of the cyclophosphamide (CPA) based and doxorubicin (DOX) based anticancer treatments including CHOP regimen (CPA, doxorubicin, vincristine, and prednisone) while reducing cardiotoxicity associated with DOX.

### CROSS-REFERENCE TO RELATED APPLICATIONS

This application claims the benefit of U.S. Provisional Patent Applications No. 62/984,994, filed Mar. 4, 2020, incorporated by reference herein in its entirety.

### STATEMENT OF GOVERNMENT SUPPORT

This invention was made with government support under Grant Number GM107058 awarded by the National Institutes of Health. The government has certain rights in the invention.

### REFERENCE TO SEQUENCE LISTING SUBMITTED ELECTRONICALLY

The sequence listing contained in the file named “115834-5024-WO_seglisting.txt”, created on Mar. 4, 2021, and having a size of 1.89 kilobytes, has been submitted electronically herewith via EFS-Web, and the contents of the txt file are hereby incorporated by reference in their entirety.

### FIELD

The disclosure relates generally to the field of medicine, cancer biology, and in particular novel compounds/agents which can enhance the therapeutic efficacy of cyclophosphamide-based and doxorubicin-based chemotherapy, pharmaceutical compositions comprising such novel compounds/agents, and methods of making and use thereof.

### BACKGROUND

Cyclophosphamide (CPA) and doxorubicin (DOX) are widely used chemotherapeutics in many diseases, spanning from immune disorders to cancer. While CPA requires hepatic metabolism mediated by cytochrome P450 (CYP) 2B6, DOX intercalates into the DNA of rapidly dividing cells and is associated with generation of reactive oxygen species (ROS) leading to off-target cardiotoxicity. Unfortunately, a significant number of patients remain uncured due to development of drug resistance and/or intolerable toxicities. There is a need for further optimization of current treatment regimens.

### SUMMARY

In one embodiment, the disclosure provides a compound of formula (I), or a pharmaceutically acceptable salt, solvate, hydrate, cocrystal, or prodrug thereof:

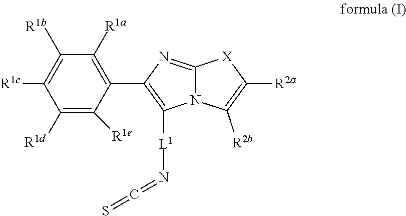

wherein in formula (I):

R^{1a}, R^{1b}, R^{1c}, R^{1d}, R^{1e}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl, and wherein R^{1a} and R^{1b}, R^{1b} and R^{1c}, R^{1c} and R^{1d}, R^{1d} and R^{1e}, and/or R^{2a} and R^{2b} are optionally joined together to form an optionally substituted aryl ring;

X is O or S;

L¹ is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and

R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In one embodiment, the disclosure provides a compound of formula (1), or a pharmaceutically acceptable salt, solvate, hydrate, cocrystal, or prodrug thereof:

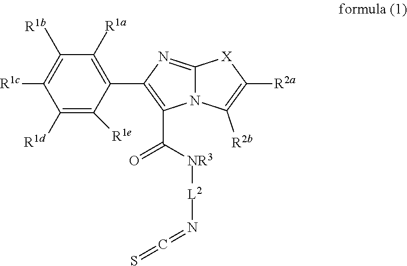

wherein in formula (1):

R³ is H or optionally substituted alkyl; and

L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

In one embodiment, R^{1a}, R^{1b}, R^{1c}, R^{1d}, and R^{1e} are each independently selected from H, halo, and trifluoromethyl. In some embodiments, R^{1c} is selected from F, Cl, and trifluoromethyl. In some embodiments, R^{1a}, R^{1b}, R^{1c}, R^{1d}, and R^{1e} are each H. In some embodiments, R^{1b} and R^{1c} are joined together to form an optionally substituted aryl ring. In one embodiment, the disclosure provides a compound of formula (2), or a pharmaceutically acceptable salt, solvate, hydrate, cocrystal, or prodrug thereof:

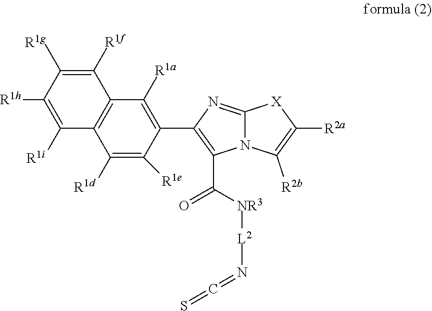

wherein in formula (2):

R^{1f}, R^{1g}, R^{1h}, and R¹ⁱ are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heterocyclyl;

R³ is H or optionally substituted alkyl; and

L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

In one embodiment, R^{1a}, R^{1d}, R^{1e}, R^{1f}, R^{1g}, R^{1h}, and R¹ⁱ are each H. In one embodiment, R^{2a} and R^{2b} are each H. In some embodiments, X is O. In one embodiment, X is S. In one embodiment, L² is selected from optionally substituted C₂-C₄ alkyl, optionally substituted cycloalkyl, and optionally substituted heterocyclyl.

In one embodiment, L² is selected from

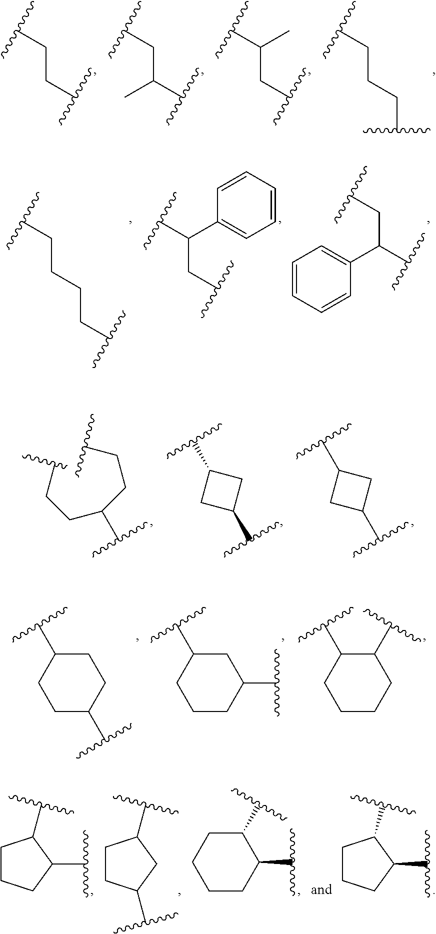

In one embodiment, the disclosure provides a compound of formula (11) or (12), or a pharmaceutically acceptable salt, solvate, hydrate, cocrystal, or prodrug thereof:

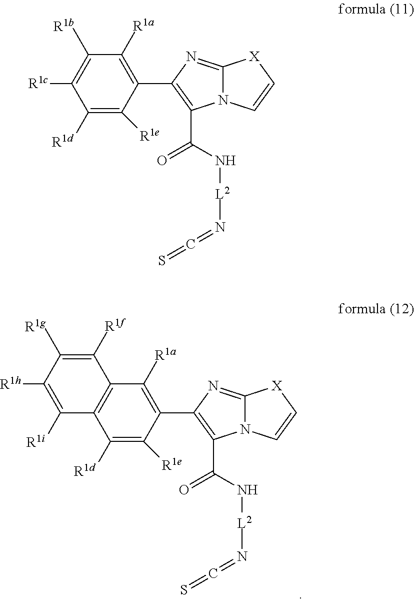

In one embodiment, the disclosure provides a compound of formula (21) or (22), or a pharmaceutically acceptable salt, solvate, hydrate, cocrystal, or prodrug thereof:

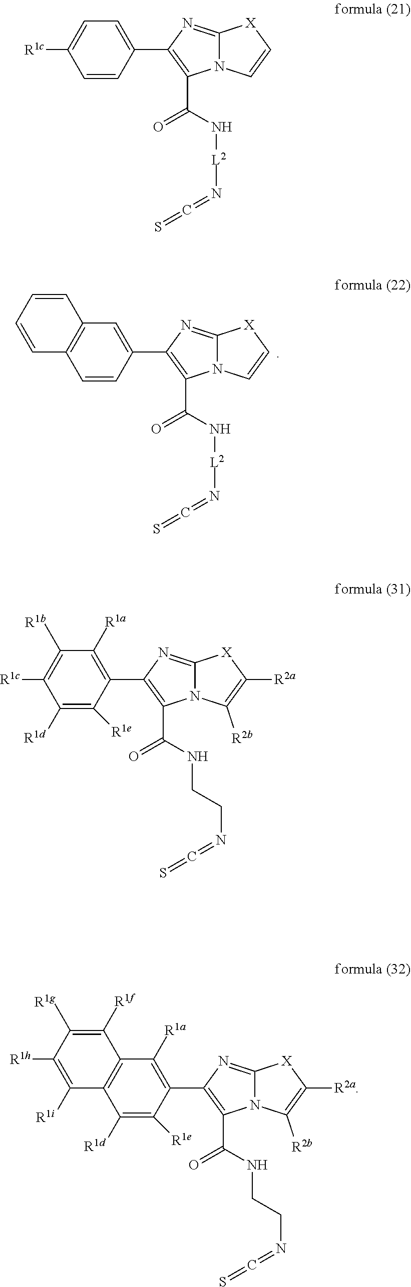

In one embodiment, R^{1c} is selected from F, Cl, and trifluoromethyl.

In one embodiment, the disclosure provides a compound of formula (31) or (32), or a pharmaceutically acceptable salt, solvate, hydrate, cocrystal, or prodrug thereof: In one embodiment, the disclosure provides a compound of formula 1001 to 1096, or a pharmaceutically acceptable salt, solvate, hydrate, cocrystal, or prodrug thereof: In one embodiment, the disclosure provides a compound of formula 2001 to 2024, or a pharmaceutically acceptable salt, solvate, hydrate, cocrystal, or prodrug thereof:

In one embodiment, the compound is

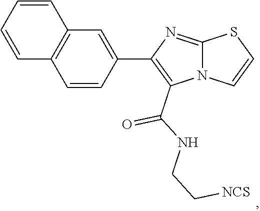

or a pharmaceutically acceptable salt, solvate, hydrate, cocrystal, or prodrug thereof.

In one embodiment, the compound is a hCAR activator. In some embodiments, the compound is a Nrf2 activator. In some embodiments, the compound is a dual hCAR and Nrf2 activator.

In one embodiment, the disclosure provides pharmaceutical composition comprising a compound disclosed herein, or a pharmaceutically acceptable salt, solvate, hydrate, cocrystal, or prodrug thereof, and a physiologically compatible carrier medium.

In one embodiment, the disclosure provides a method of treating a disease alleviated by activating hCAR in a patient in need thereof, the method comprising administering to the patient a therapeutically effective amount of a compound disclosed herein, or a pharmaceutically acceptable salt, solvate, hydrate, cocrystal, or prodrug thereof.

In one embodiment, the disclosure provides a method of treating a disease alleviated by activating Nrf2 in a patient in need thereof, the method comprising administering to the patient a therapeutically effective amount of a compound disclosed herein, or a pharmaceutically acceptable salt, solvate, hydrate, cocrystal, or prodrug thereof.

In one embodiment, the disclosure provides a method of treating a disease alleviated by activating hCAR and Nrf2 in a patient in need thereof, comprising administering to the patient a therapeutically effective amount of a compound disclosed herein, or a pharmaceutically acceptable salt, solvate, hydrate, cocrystal, or prodrug thereof.

In one embodiment, CYP2B6 is selectively induced over CYP3A4.

In one embodiment, the method further comprising administering to the patient a therapeutically effective amount of cyclophosphamide (CPA) and doxorubicin (DOX), or a pharmaceutically acceptable salt, solvate, hydrate, cocrystal, or prodrug thereof.

In one embodiment, CPA and DOX is administered as part of the CHOP regimen (CPA, doxorubicin, vincristine, and prednisone).

In one embodiment, co-administration of a compound of any one of claims 1-25, or a pharmaceutically acceptable salt, solvate, hydrate, cocrystal, or prodrug thereof, CPA, and DOX, promotes the formation of therapeutically active CPA metabolite 4-OH-CPA and decreased cleaved caspase-3 expression.

In one embodiment, the compound, or pharmaceutically acceptable salt, solvate, hydrate, cocrystal, or prodrug thereof, is administered in a dosage unit form.

In one embodiment, the dosage unit form comprises a physiologically compatible carrier medium.

In one embodiment, the disease is cancer.

In one embodiment, the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In one embodiment, the cancer is a triple negative breast cancer (TNBC).

### BRIEF DESCRIPTION OF THE DRAWINGS

FIG. 1 shows a general method for synthesis of compounds of Formula (I), including exemplary compounds prepared using same.

FIG. 2a-FIG. 2b show the ¹H NMR spectrum (FIG. 2a) and the ¹³C NMR spectrum (FIG. 2b) of N-(2-Isothiocyanatoethyl)-6-(naphthalen-2-yl)imidazo[2,1-b]oxazole-5-carboxamide (DL7009).

FIG. 3a-FIG. 3b show the ¹H NMR spectrum (FIG. 3a) and the ¹³C NMR spectrum (FIG. 3b) of N-(2-Isothiocyanatopropyl)-6-(naphthalen-2-yl)imidazo[2,1-b]oxazole-5-carboxamide (DL7092).

FIG. 4a-FIG. 4b show the ¹H NMR spectrum (FIG. 4a) and the ¹³C NMR spectrum (FIG. 4b) of N-(1-Isothiocyanatopropan-2-yl)-6-(naphthalen-2-yl)imidazo[2,1-b]oxazole-5-carboxamide (DL7091).

FIG. 5a-FIG. 5b show the ¹H NMR spectrum (FIG. 5a) and the ¹³C NMR spectrum (FIG. 5b) of N-(2-Isothiocyanato-2-phenylethyl)-6-(naphthalen-2-yl)imidazo[2,1-b]oxazole-5-carboxamide (DL70552).

FIG. 6a-FIG. 6b show the ¹H NMR spectrum (FIG. 6a) and the ¹³C NMR spectrum (FIG. 6b) of Tert-butyl N-(2-isothiocyanato-1-phenylethyl)-6-(naphthalen-2-yl)imidazo[2,1-b]oxazole-5-carboxamide (DL70562).

FIG. 7a-FIG. 7b show the ¹H NMR spectrum (FIG. 7a) and the ¹³C NMR spectrum (FIG. 7b) of (4-Isothiocyanatopiperidin-1-yl)(6-(naphthalen-2-yl)imidazo[2,1-b]oxazol-5-yl)methanone (DL7077).

FIG. 8a-FIG. 8b show the ¹H NMR spectrum (FIG. 8a) and the ¹³C NMR spectrum (FIG. 8b) of N-((1r,3r)-3-Isothiocyanatocyclobutyl)-6-(naphthalen-2-yl)imidazo[2,1-b]oxazole-5-carboxamide (DL7102).

FIG. 9a-FIG. 9b show the ¹H NMR spectrum (FIG. 9a) and the ¹³C NMR spectrum (FIG. 9b) of N-(4-Isothiocyanatocyclohexyl)-6-(naphthalen-2-yl)imidazo[2,1-b]oxazole-5-carboxamide (DL7086).

FIG. 10a-FIG. 10b show the ¹H NMR spectrum (FIG. 10a) and the ¹³C NMR spectrum (FIG. 10b) of N-((1R,2R)-2-Isothiocyanatocyclohexyl)-6-(naphthalen-2-yl)imidazo[2,1-b]oxazole-5-carboxamide (DL7096).

FIG. 11a-FIG. 11b show the ¹H NMR spectrum (FIG. 11a) and the ¹³C NMR spectrum (FIG. 11b) of N-((1S,2S)-2-Isothiocyanatocyclopentyl)-6-(naphthalen-2-yl)imidazo[2,1-b]oxazole-5-carboxamide (DL7097).

FIG. 12a-FIG. 12b show the ¹H NMR spectrum (FIG. 12a) and the ¹³C NMR spectrum (FIG. 12b) of N-(3-Isothiocyanatopropyl)-6-(naphthalen-2-yl)imidazo[2,1-b]oxazole-5-carboxamide (DL7087).

FIG. 13a-FIG. 13b show the ¹H NMR spectrum (FIG. 13a) and the ¹³C NMR spectrum (FIG. 13b) of N-(4-Isothiocyanatobutyl)-6-(naphthalen-2-yl)imidazo[2,1-b]oxazole-5-carboxamide (DL7101).

FIG. 14a-FIG. 14b show the ¹H NMR spectrum (FIG. 14a) and the ¹³C NMR spectrum (FIG. 14b) of N-(2-Isothiocyanatoethyl)-6-(naphthalen-2-yl)imidazo[2,1-b]thiazole-5-carboxamide (DL7076).

FIG. 15a-FIG. 15b show the ¹H NMR spectrum (FIG. 15a) and the ¹³C NMR spectrum (FIG. 15b) of N-(2-Isothiocyanatoethyl)-6-phenylimidazo[2,1-b]thiazole-5-carboxamide (DL7134).

FIG. 16a-FIG. 16b show the ¹H NMR spectrum (FIG. 16a) and the ¹³C NMR spectrum (FIG. 16b) of 6-(4-Fluorophenyl)-N-(2-isothiocyanatoethyl)imidazo[2,1-b]thiazole-5-carboxamide (DL7135).

FIG. 17a-FIG. 17b show the ¹H NMR spectrum (FIG. 17a) and the ¹³C NMR spectrum (FIG. 17b) of 6-(4-Chlorophenyl)-N-(2-isothiocyanatoethyl)imidazo[2,1-b]thiazole-5-carboxamide (DL7128).

FIG. 18a-FIG. 18b show the ¹H NMR spectrum (FIG. 18a) and the ¹³C NMR spectrum (FIG. 18b) of N-(2-Isothiocyanatoethyl)-6-(4-(trifluoromethyl)phenyl)imidazo[2,1-b]thiazole-5-carboxamide (DL7127).

FIG. 19a-FIG. 19b show the ¹H NMR spectrum (FIG. 19a) and the ¹³C NMR spectrum (FIG. 19b) of N-(2-Isothiocyanatopropyl)-6-(naphthalen-2-yl)imidazo[2,1-b]thiazole-5-carboxamide (DL7139).

FIG. 20a-FIG. 20b show the ¹H NMR spectrum (FIG. 20a) and the ¹³C NMR spectrum (FIG. 20b) of N-(1-Isothiocyanatopropan-2-yl)-6-(naphthalen-2-yl)imidazo[2,1-b]thiazole-5-carboxamide (DL7140).

FIG. 21a-FIG. 21b show the ¹H NMR spectrum (FIG. 21a) and the ¹³C NMR spectrum (FIG. 21b) of N⁵-(2,3-Dihydro-1H-inden-2-yl)-N⁶-(2-isothiocyanatoethyl)imidazo[2,1-b]thiazole-5,6-dicarboxamide (DL7021).

FIG. 22a-FIG. 22c show the structure (FIG. 22a), all curve (FIG. 22b), and EC₅₀ curve (FIG. 22c) of DL7055-2. Values: Emax=6.2, EC₅₀ (μM)=19±0.08.

FIG. 23a-FIG. 23c show the structure (FIG. 23a), all curve (FIG. 23b), and EC₅₀ curve (FIG. 23c) of DL7056-2. Values: EC₅₀ (μM)=>100.

FIG. 24a-FIG. 24c show the structure (FIG. 24a), all curve (FIG. 24b), and EC₅₀ curve (FIG. 24c) of DL7077. Values: Emax=1.8, EC₅₀ (μM)=4.8±0.21.

FIG. 25a-FIG. 25c show the structure (FIG. 25a), all curve (FIG. 25b), and EC₅₀ curve (FIG. 25c) of DL7086. Values: EC₅₀ (μM)=ND.

FIG. 26a-FIG. 26c show the structure (FIG. 26a), all curve (FIG. 26b), and EC₅₀ curve (FIG. 26c) of DL7087. Values: Emax=1.27, EC₅₀ (μM)=1.1±0.43.

FIG. 27a-FIG. 27c show the structure (FIG. 27a), all curve (FIG. 27b), and EC₅₀ curve (FIG. 27c) of DL7091. Values: Emax=3.6, EC₅₀ (μM)=4.1.

FIG. 28a-FIG. 28c show the structure (FIG. 28a), all curve (FIG. 28b), and EC₅₀ curve (FIG. 28c) of DL7092. Values: Emax=6.0, EC₅₀ (μM)=32.

FIG. 29a-FIG. 29c show the structure (FIG. 29a), all curve (FIG. 29b), and EC₅₀ curve (FIG. 29c) of DL7096. Values: Emax=1.1, EC₅₀ (μM)=0.33.

FIG. 30a-FIG. 30b show the structure (FIG. 30a) and all curve (FIG. 30b) of DL7097.

FIG. 31a-FIG. 31b show the structure (FIG. 31a) and all curve (FIG. 31b) of DL7102.

FIG. 32a-FIG. 32c show the structure (FIG. 32a), all curve (FIG. 32b), and EC₅₀ curve (FIG. 32c) of DL7101. Values: Emax=1.6, EC₅₀ (μM)=0.42.

FIG. 33a-FIG. 33c show the structure (FIG. 33a), all curve (FIG. 33b), and EC₅₀ curve (FIG. 33c) of DL7009. Values: Emax=1.4, EC₅₀ (μM)=0.93.

FIG. 34a-FIG. 34c show the structure (FIG. 34a), all curve (FIG. 34b), and EC₅₀ curve (FIG. 34c) of DL7076. Values: Emax=2.5, EC₅₀ (μM)=3.9.

FIG. 35a-FIG. 35c show the structure (FIG. 35a), all curve (FIG. 35b), and EC₅₀ curve (FIG. 35c) of DL7021.

FIG. 36a-FIG. 36c shows the screening, design, and strategy for the discovery of compound hybrids of the disclosure. FIG. 36a shows the design and strategy for hCAR-Nrf2 dual activators compounds 1-13. FIG. 36b shows the conditions and reagents for the exemplary synthesis of compounds 1-13. FIG. 36c shows a flowchart of an exemplary method for screening hybrid compounds.

FIG. 37a-FIG. 37c show experimental data demonstrating the results from screening using luciferase reporter assays to identify DL7076 as a hCAR and Nrf2 dual activator. After a transient transfection, HepG2 cells were treated with multiple compound hybrids in indicated concentrations for screening. DL7076 was identified for its potency and efficacy in both hCAR and Nrf2 luciferase assays. FIG. 37a shows a graph of experimental results from a hCAR agonist assay. FIG. 37b shows a graph of experimental results from a Nrf2 agonist assay. FIG. 37c shows structures of compounds of the disclosure that were tested. Results are expressed as mean±SD (n=3).

FIG. 38a shows experimental data demonstrating that DL7076 induces CYP2B6 expression in human primary hepatocytes. Luciferase activity of HepG2 cells transfected transiently and treated with a vehicle control (0.1% DMSO), RIF (10 μM), CITCO (1 μM), and DL7076 (1 μM, 5 μM, and 10 μM) for 24 hours was measured. FIG. 38b shows experimental data from human primary hepatocytes prepared from liver donor #159 were infected with Ad/EYFP-hCAR for 24 hours. Following the infection, cells were treated with a vehicle control (0.1% DMSO), PB (1 mM), and DL7076 (10 μM) for 6 hours and EYFP-hCAR was assessed for nuclear localization. FIGS. 38c-38h show experimental data from human hepatocytes prepared from liver donors #155 (FIGS. 38c-38e) and #156 (FIGS. 38f-38h) treated with a vehicle control, RIF (10 μM), CITCO (1 μM), and DL7076 (1 μM, 5 μM, and 10 μM) for 24 hours. Expression of CYP2B6 and CYP3A4 mRNA and protein were measured using real-time PCR and Western blotting assays. RNA was isolated and protein was harvested for CYP2B6 and CYP3A4 expression. Induction of genes was analyzed using qPCR. All values are presented as fold change vs. vehicle control. Results are expressed as mean±SD (n=3). ***, P<0.001.

FIG. 39a-FIG. 39g show experimental data demonstrating that DL7076 modulates Nrf2 and induces HO-1 in a tissue specific manner. H9c2 (FIGS. 39a and 39b) cardiomyocytes and two TNBC cell lines, BT549 (FIGS. 39c and 39d) and MDA-MB-231 (FIGS. 39e and 39f), were treated with a vehicle control (0.1% DMSO), SFN (2.5 μM), or DL7076 (1 μM, 5 μM, 10 μM) for 24 hours. Expression of HO-1 mRNA and protein were measured using real-time PCR and Western blotting assays. FIG. 39g shows experimental data demonstrating that H9c2 cardiomyocytes were treated with a vehicle control, SFN (2.5 μM), or DL7076 (1 μM, 5 μM, 10 μM) for 24 hours, then lysed and extraction was preformed to get the nuclear fraction. RNA was isolated and protein was harvested for HO-1 and Nrf2 expression. Induction of genes was analyzed using qPCR. All values are presented as fold change vs. vehicle control. Statistical significance was determined using a One-Way ANOVA with a Bonferroni post test. Results are expressed as mean±SD. *, P<0.05, ***, P<0.001.

FIG. 40a-FIG. 40d show experimental data demonstrating that DL7076 enhances 4-OH-CPA formation and TNBC cell cytotoxicity. FIG. 40a shows a schematic illustration of the coculture system containing HPH and BT549. FIG. 40b shows a graph of experimental data demonstrating that in the coculture model, CPA cytotoxicity in BT549 cells was measured in the presence and absence of CITCO (1 μM) or DL7076 (10 μM). FIG. 40c shows a graph of experimental data demonstrating the effects of dual activation on the concentration-dependent formation of 4-OH-CPA under treatments previously described. FIG. 40d shows a graph of experimental data demonstrating the effects of dual activation on 4-OH-CPA formation as function of time under treatments described in FIG. 40c. Following that incubation, coverslips with BT549 cells were inserted and co-administered with CPA (250 μM, 500 μM, and 1000 μM) for 48 hours. 6% CCK8 was used to determine viability. Statistical significance was determined by vehicle control vs. treatment group using a Two-Way ANOVA with a Bonferroni post test. **, P<0.01, ***, P<0.001.

FIG. 41a-FIG. 41h show experimental data demonstrating that DL7076 protects H9c2 cardiomyocytes but not TNBC cells. H9c2 and TNBC cells were pretreated with vehicle control (0.1% DMSO), SFN (2.5 μM), or DL7076 (1 μM, 5 μM, 10 μM) for 2 hours, followed by a cotreatment with DOX (2.5 μM) for 22 hours. Recovery of DOX-induced toxicity in H9c2 (FIG. 41a), BT549 (FIG. 41b), and MDA-MB-231 (FIG. 41c) cell was demonstrated in a concentration dependent manner. Western blotting assays were performed to measure the HO-1 and cleaved-caspase 3 protein in H9c2 (FIG. 41d), BT549 (FIG. 41e), and MDA-MB-231 (FIG. 41f) cells were harvested. Densitometry of H9c2 protein was normalized to that of β-actin. RNA was isolated and protein was harvested for HO-1 and Nrf2 expression. Induction of genes was analyzed using qPCR. All values are presented as fold change vs. vehicle control. Statistical significance was determined using a One-Way ANOVA with a Bonferroni post test. *, P<0.05, ***, P<0.001. FIGS. 41g and 41h shows graphs of experimental data demonstrating HO-1 relative mRNA induction (FIG. 41g) and percent cell viability (FIG. 41h) for IPSC-derived cardiomyocytes treated with control, SFN (2.5 μM), or DL7076 (1 μM, 5 μM, 10 μM).

FIG. 41a-FIG. 41c show experimental data demonstrating that DL7076 reduces oxidative stress selectively in H9c2. H9c2 (FIG. 41a), BT549 (FIG. 41b), and MDA-MB-231 (FIG. 41c) cells were pretreated for 2 hours with 0.1% DMSO, SFN (2.5 μM), or DL7076 (5 μM) and then treated again with 0.1% DMSO, SFN (2.5 μM), or DL7076 (5 μM) in the presence and absence of DOX (1 μM, 2.5 μM, and 5 μM) for 6 hours. Oxidative stress was assessed using 20 μM 2′,7′-dichlorofluorescein diacetate (DFDA) at 37° C. and 5% CO₂ for 30 minutes. The oxidation of DFDA to dichlorofluorescein was quantified using NIS-Element Analysis with Brightspot Detection and normalized to the vehicle control. Representative images via fluorescence microscopy are shown. Statistical significance was determined by vehicle control vs. treatment group using a Two-Way ANOVA with a Bonferroni post test. *, P<0.05, ***, P<0.001.

FIG. 42a-FIG. 42e show experimental data demonstrating that DL7076 enhances CPA cytotoxicity in BT549 cells and hinders DOX-induced toxicity in H9c2 cells. FIG. 42a shows a schematic illustration of the multi-organ coculture system containing BT549, h9c2, and HPH cells. FIGS. 42b-42e show experimental data of results from the coculture model. CPA/DOX cytotoxicity in BT549 (FIG. 42b) and H9c2 (FIG. 42c) cells was measured in the presence and absence of CITCO (1 μM) or DL7076 (10 μM). BT549 (FIG. 42d) and H9c2 (FIG. 42e) cells were removed from the co-culture and fixed with 4% paraformaldehyde, followed by an immune-staining for phosphorylation at serine-139 position of histone H2AX. Relative H2AX phosphorylation was quantified using NIS-Element Analysis using Brightspot Detection and normalized to the vehicle control. Representative images via fluorescence microscopy were shown. FITC channel was modified to red for viewers clarity. All values are presented as fold change vs. control. Statistical significance was determined by vehicle control vs. treatment group using Two-Way ANOVA with a Bonferroni post test. *,P<0.05, **, P<0.01, ***P<0.001.

FIG. 43 shows a schematic illustration of DL7076 enhancement of CPA/DOX anticancer activity in target cells (BT549 and MDA-MB-231) and reduction of off-target toxicity in off target cells (H9c2).

### DETAILED DESCRIPTION

The CYP2B6 and CYP3A4 are two primary enzymes in the human cytochrome P450 super family responsible for xenobiotic metabolism. The CYP2B6 is responsible for the metabolic conversion of CPA, a prodrug, to the pharmacologically active 4-hydroxylcyclophosphamide (4-OH-CPA). On the other hand, CYP3A4 can convert CPA to its inactive metabolite N-dechloroethyl-CPA (N-DCE-CPA) and the toxic byproduct chloroacetaldehyde. The human constitutive androstane receptor (hCAR, NR113) and the pregnane X receptor (PXR, NR112) are key modulators governing the inductive expression of CYP2B6. The selective activation of hCAR over PXR preferentially induces the expression of hepatic CYP2B6 over CYP3A4 and increases the formation of 4-OH-CPA without concomitant increasing the non-therapeutic metabolites. The selective transcription of CYP2B6 over CYP3A4 by hCAR may have clinical relevance with respect to drugs that are predominantly metabolized by CYP2B6. The activators of hCAR may function as co-administrated facilitators for such biotransformation. CYP2B6 selectively upregulated by hCAR, provides an attractive approach for improving CPA-based therapeutics.

The process of enhancement of the anticancer activity is synergetic when the anticancer activity of the combination is in excess of the simple addition of the activity of the two separate substances. The hCAR activity induction component of the mixture is a synergist.

A New Chemical Entity (NCE), DL5016 that acts as a selective hCAR activator to facilitate CPA-based chemotherapy for hematologic malignancies was developed. CPA, a DNA-alkylating prodrug, is extensively used in the treatment of various solid cancers and hematologic malignancies. However, its harsh side effects contribute significantly to patient morbidity and mortality. DL5016 is a useful addition to CPA-containing regimens, as hCAR activation potentiates the benefits of CPA, without altering its side effect profile. CPA is a mainstay of numerous drug combinations, most importantly the CHOP (CPA, doxorubicin, vincristine, and prednisone) regimen, which is the first-line chemotherapy for non-Hodgkin's lymphoma and a number of other hematologic malignancies. Mechanistically, the CYP2B6 enzyme expressed in hepatocytes converts CPA to pharmacologically active 4-hydroxyl-CPA (4-OH-CPA). CYP2B6 is selectively upregulated by hCAR, providing an attractive approach for improving CPA-based therapeutics.

CPA-based chemotherapy continues to be the mainstay of many front-line chemotherapeutic regimens for the CI treatment of non-Hodgkin lymphoma, chronic lymphocytic leukemia, triple negative breast cancers, and other solid tumors. As a prodrug, CPA relies heavily on hepatic CYP2B6-mediated bioactivation to generate the active alkylating moiety before exhibiting chemotherapeutic effects. Alternatively, CYP3A4 can convert CPA to its inactive metabolite N-dechloroethyl-CPA (N-DCE-CPA) and the toxic byproduct chloroacetaldehyde. It is shown that activation of hCAR preferentially induces the expression of CYP2B6 in the liver, without concurrent augmentation of its nontherapeutic metabolites.

Breast cancer is a heterogeneous malignancy, leading to variable prognoses based on clinical classification, stage of the disease, and the choice of treatment. In 2019, 331,530 new cases of breast cancer are estimated, while 41,760 women are expected to die in the United States. Triple negative breast cancer (TNBC) accounts for 10-20% of all breast cancers and is biologically more aggressive, due to the lack of estrogen and progesterone receptor, and human epidermal growth factor receptor 2 expression. TNBC is generally unresponsive to hormonal or molecular therapeutics making conventional cytotoxic chemotherapy the mainstay treatment. Cyclophosphamide (CPA) and doxorubicin (DOX) are commonly used as part of the backbone treatment of TNBC. Although initially responsive to this combination, a significant portion of TNBC patients succumb to drug resistance or intolerable side toxicities leading to the discontinuation of their vital regimen. The need for further optimization of the current treatment regimen is evident, with the aim to improve target cell cytotoxicity and decrease severe side toxicities.

CPA, an alkylating prodrug, is used in a variety of diseases, spanning from immune system disorders to cancer. Once administered, CPA gets bioactivated by hepatic drug-metabolism enzymes to form the rate-limiting metabolite 4-hydroxycyclophosphamide (4-OH-CPA). The primary cytochrome P450 (CYP) which mediates this conversion is CYP2B6 and to a lesser extent, CYP3A4. Through CYP3A4-mediated metabolism, a portion of the parent CPA can be converted into both an inactive byproduct, dechloroethyl-CPA, and a neurotoxic byproduct, chloroacetyldehyde. However, once oxidized by CYP2B6, 4-OH-CPA can tautomerize to aldophosphamide, followed by spontaneous β-elimination which can produce the phosphoramide mustard. That nitrogen mustard can alkylate into the nucleophilic groups of DNA in target cancer cells, leading to cell death via the apoptosis pathway.

The constitutive androstane receptor (CAR, NRli3) is a master hepatic xenobiotic regulator and the predominant modulator of the induction of CYP2B6. Previous work has shown selective activation of CAR leads to the preferential induction of CYP2B6, thereby, enhancing the formation of the 4-OH-CPA in isolated human primary hepatocytes (HPH), leading to increased conversion of the parent CPA to 4-OH-CPA and enhanced cytotoxicity of CPA towards HL-60 human leukemia cells and SU-DHL-4 lymphoma cells in a coculture system.

DOX, an anti-neoplastic agent, is widely used and considered highly effective in combination with CPA as the backbone treatment of TNBC. However, DOX is limited by its dosage-dependent accumulative cardiotoxicity, which can aid in the progression of cardiomyopathy and congestive heart failure (CHF). This DOX-induced cardiotoxicity has been associated with many mechanisms including oxidative stress, autophagy, lipid peroxidation, iron metabolism, among others. The production of reactive oxygen species (ROS) and mitochondrial dysfunction caused by DOX being reduced by mitochondrial nicotinamide adenine dinucleotide phosphate-oxidase (NADPH oxidase) to a semi-quinone free radical has been widely accepted as a central mediator in cardiotoxicity. These free radicals can interconvert in the presence of oxygen to form superoxide radicals in the mitochondria of cardiomyocytes, leading to apoptosis. DOX-induced ROS in cardiomyocytes contributes to the progressive heart failure in patients.

The nuclear factor erythroid-2 related factor 2 (Nrf2) is a key transcription factor in the antioxidant pathway and activation can lead to the induction of target genes, such as heme oxygenase-1 (HO-1), which can decrease DOX-induced ROS seen in cardiomyocytes. Under basal conditions, Nrf2 is bound to a regulatory protein, Kelch-like ECH-associated protein (Keap1) and destine for proteasomal degradation. However, when this interaction is disrupted, through modification of reactive cysteine residues of Keap1, Nrf2 can translocate into the nucleus leading to the induction of protective antioxidant enzymes, such as HO-1. Previous work has shown that selective activation of CAR can increase the cytotoxicity of CPA in target cells without enhancing toxicity in cardiomyocytes.

Currently, CPA, an alkylator, and DOX, an anthracycline, are a significant part of the standard backbone treatment for triple negative breast cancer (TNBC). Despite the evolution of the application of this regimen, a significant number of initially responsive patients still succumb to their disease due to discontinuation of their vital chemotherapeutic treatment due to drug resistance and/or intolerable toxicities, such as cardiotoxicity. A common component of TNBC treatment includes the alkylating prodrug, CPA, which requires hepatic oxidation catalyzed by CYP2B6 to the pharmacologically active metabolite, 4-OH-CPA. In addition to the alkylator, the anthracycline, DOX, is responsible for the majority of the cardiotoxicity related to the CPA/DOX containing regimens. It was previously demonstrated that activation of CAR, the nuclear receptor, leads to the selective induction of CYP2B6 and subsequent formation of 4-OH-CPA. Additionally, it has been demonstrated that activation of Nrf2 leads to a reduction in DOX-induced cardiotoxicity through the modulation of ROS in cardiomyocytes.

Cardiotoxicity is the major concern clinically when an anthracycline, DOX, is given to TNBC patients. It is estimated that 10-25% of patients experience cardiotoxicity leading to a cardiac event after anthracycline treatment. Secondary to malignancy, cardiovascular disease is the leading cause of mortality. DOX-induced cardiotoxicity is mediated through multifaceted mechanisms, including the generation of free radicals, mitochondrial dysfunction, oxidative stress, iron metabolism, and more. Although there is varied mechanism for cardiotoxicity, the generation of reactive oxygen species is widely recognized to contribute to the overall toxicity of the anthracycline treatment.

As described herein, the impact of a selective CAR and Nrf2 dual activator on the anticancer activity and off-target toxicity of CPA and DOX-containing treatment regimens for the treatment of TNBC was examined. To fully illuminate the impact of a dual activator, a novel multi-organ co-culture system was utilized. This system provides a shared cellular environment that includes metabolically competent HPH, target TNBC cells, and off-target cardiomyocytes, allowing for further evaluation of the impact of hepatic-mediated anticancer activity of CPA and DOX while monitoring off-target toxicity concurrently. Also described herein is experimental evidence that the inclusion of a selective dual activator of CAR and Nrf2 significantly enhances the efficacy of CPA and DOX in the TNBC treatment while simultaneously easing DOX-induced toxicity in cardiomyocytes.

### Definitions

As used in the preceding sections and throughout the rest of this specification, unless defined otherwise, all technical and scientific terms used herein have the same meaning as is commonly understood by one of skill in the art to which this disclosure belongs. All patents and publications referred to herein are incorporated by reference in their entireties.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or P03(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or P03(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R² of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or P03(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R, —C(O)OR, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R, —C(O)OR, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocyclo,” “heterocycle,” or “heterocyclic ring,” refers to an unsubstituted or substituted stable 5- to 7-membered monocyclic ring system which may be saturated or unsaturated, and which consists of carbon atoms and from one to four heteroatoms selected from N, O or S, and wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but are not limited to, piperidinyl, piperazinyl, oxopiperazinyl, oxopiperidinyl, oxopyrrolidinyl, oxoazepinyl, azepinyl, pyrrolyl, pyrrolidinyl, benzothiophene, chromone, benzopyrene, benzopyrone, furanyl, thienyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isooxazolyl, isoxazolidinyl, morpholinyl, thiazolyl, thiazolidinyl, isothiazolyl, thiadiazolyl, tetrahydropyranyl, thiamorpholinyl, thiamorpholinylsulfoxide, thiamorpholinylsulfone, and oxadiazolyl.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO³(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula.

“Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S′). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

The terms “substituted” or “optionally substituted” refer to chemical moieties, wherein one or more hydrogen atoms may be replaced by a halogen atom, a NH₂, SH, NO₂ or OH group, or by an alkyl, alkenyl, alkanoyl, heteroalkyl, aryl, heteroaryl, cycloalkyl or heterocycle group as defined herein. The last-mentioned groups may be optionally substituted.

The terms “subject” and “patient” are used interchangeably herein to refer to a warm blooded animal such as a mammal, preferably a human, or a human child, which is afflicted with, or has the potential to be afflicted with one or more diseases and/or conditions described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the terms “treat,” “treatment,” and/or “treating” may refer to the management of a disease, disorder, or pathological condition, or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “combination” or “pharmaceutical combination” refers to the combined administration of the anticancer agents. Combinations of the disclosure include a compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, and at least one anti-cancer agent, such as a cyclophosphamide (CPA, an alkylating prodrug) based chemotherapy regimen and/or doxorubicin (DOX, an anti-neoplastic agent), e.g. CHOP regimen (CPA, doxorubicin, vincristine, and prednisone); which anti-cancer agents may be administered to a subject in need thereof, e.g., concurrently or sequentially.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, ALL, CLL, SLL, acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance and/or toxicity. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

“Pharmaceutically acceptable” refers to those compounds, materials, compositions, and/or dosage forms which are, within the scope of sound medical judgment, suitable for contact with the tissues of human beings and non-human animals without excessive toxicity, irritation, allergic response, or other adverse complications commensurate with a reasonable benefit/risk ratio.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

Base addition salts include those derived from inorganic bases such as ammonium and alkali and alkaline earth metal hydroxides, carbonates, bicarbonates, and the like, as well as salts derived from basic organic compounds such as aliphatic and aromatic amines, aliphatic diamines, hydroxy alkamines, and the like. Such bases useful in preparing the salts of this disclosure thus include ammonium hydroxide, potassium carbonate, sodium bicarbonate, calcium hydroxide, methylamine, diethylamine, ethylenediamine, cyclohexylamine, ethanolamine and the like.

In addition to pharmaceutically-acceptable salts, other salts are included within the scope of this disclosure. They may serve as intermediates in the purification of the compounds, in the preparation of other salts, or in the identification and characterization of the compounds or intermediates.

The pharmaceutically acceptable salts of compounds of the present disclosure can also exist as various solvates, such as with water, methanol, ethanol, dimethylformamide, ethyl acetate and the like. Mixtures of such solvates can also be prepared. The source of such solvates can be from the solvent of crystallization, inherent in the solvent of preparation or crystallization, or adventitious to such solvent. Such solvates are also within the scope of the present disclosure.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” r “having” or “including”) includes those embodiments such as, for example, an embodiment of any composition of matter, method or process that “consist of” or “consist essentially of” the described features.

All other terms used in the description of the present disclosure have their art recognized meanings.

As will be apparent to anyone skilled in the art, the compounds of the present disclosure may have one or more chiral centers, and in that case, exist in various stereoisomeric forms. The compounds of the present disclosure encompass all such optical isomers, diastereomers and enantiomers. The compounds are normally prepared as a racemic mixture or racemate and can conveniently be used as such, but individual enantiomers can be isolated or synthesized by conventional techniques if so desired. Such racemates and individual enantiomers and mixtures thereof form part of the present disclosure.

It is well known in the art how to prepare and isolate such optically active forms from a mixture of enantiomers. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994, and Jacques, J, et al. Enantiomers, Racemates, and Resolutions; Wiley: New York, 1981, each incorporated by reference herein in their entireties.

“Solvate” refers to a compound in physical association with one or more molecules of a pharmaceutically acceptable solvent.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

### Compounds

In one aspect, the disclosure relates to a compound of formula (I), or a pharmaceutically acceptable salt, solvate, hydrate, cocrystal, or prodrug thereof:

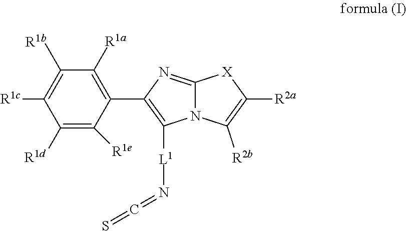

wherein in formula (I):

R^{1a}, R^{1b}, R^{1c}, R^{1d}, R^{1e}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl, and wherein R^{1a} and R^{1b}, R^{1b} and R^{1c}, R^{1c} and R^{1d}, Rid and R^{1e}, and/or R^{2a} and R^{2b} are optionally joined together to form an optionally substituted aryl ring;

X is O or S;

L¹ is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and

R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In one aspect, the disclosure relates to a compound of formula (1), or a pharmaceutically acceptable salt, solvate, hydrate, cocrystal, or prodrug thereof:

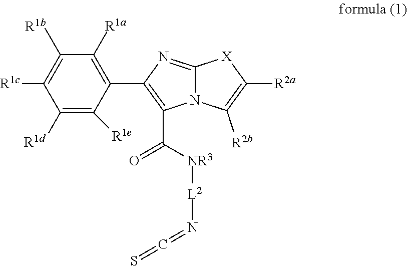

wherein in formula (1):

R³ is H or optionally substituted alkyl; and

L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

In some embodiments, R^{1a}, R^{1b}, R^{1c}, R^{1d}, and R^{1e} are each independently selected from H, halo, and trifluoromethyl. In some embodiments, R^{1a}, R^{1b}, R^{1c}, R^{1d}, and R^{1e} are each H.

In some embodiments, R^{1c} is selected from F, Cl, and trifluoromethyl.

In some embodiments, R^{1b} and R^{1c} are joined together to form an optionally substituted aryl ring.

In another aspect, the disclosure relates to a compound of formula (2), or a pharmaceutically acceptable salt, solvate, hydrate, cocrystal, or prodrug thereof:

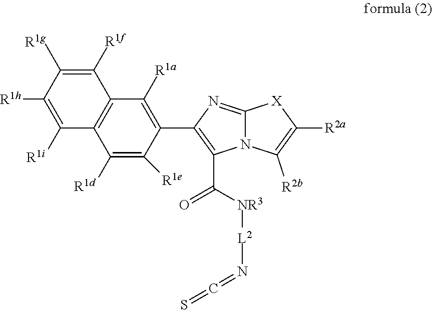

wherein in formula (2):

R^{1f}, R^{1g}, R^{1h}, and Ru are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heterocyclyl;

R³ is H or optionally substituted alkyl; and

L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

In some embodiments, R^{1a}, R^{1d}, R^{1e}, R^{1f}, R^{1g}, R^{1h}, and R¹ⁱ are each H.

In some embodiments, R^{2a} and R^{2b} are each H.

In some embodiments, R³ is H.

In some embodiments, X is O. In some embodiments, X is S.

In some embodiments, L² is selected from optionally substituted C₂-C₄ alkyl, optionally substituted cycloalkyl, and optionally substituted heterocyclyl.

In some embodiments, L² is selected from

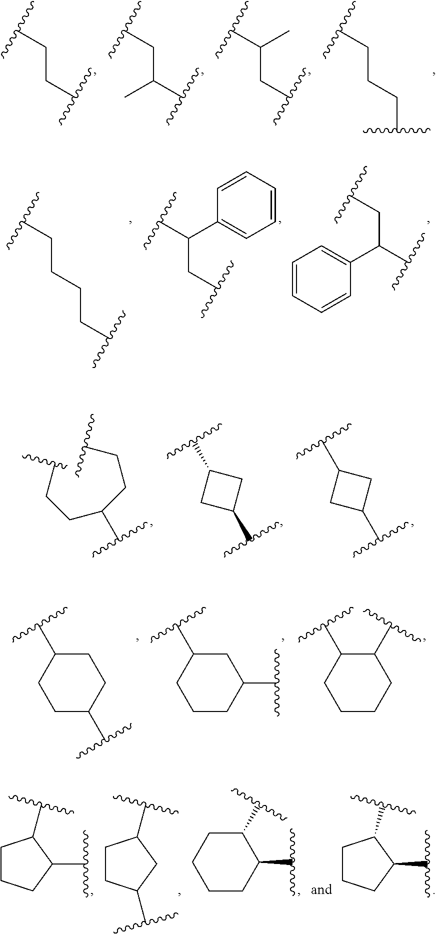

In another aspect, the disclosure relates to a compound of formula (11) or (12), or a pharmaceutically acceptable salt, solvate, hydrate, cocrystal, or prodrug thereof.

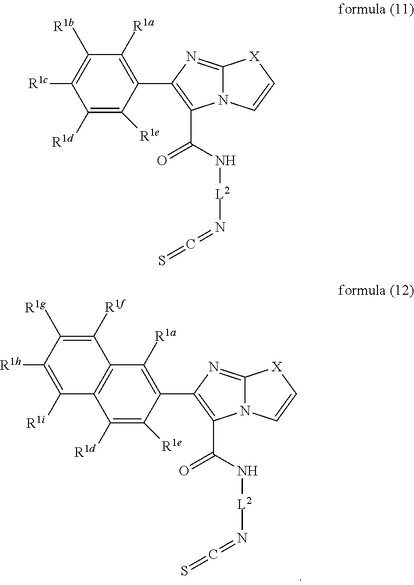

In another aspect, the disclosure relates to a compound of formula (31) or (32), or a pharmaceutically acceptable salt, solvate, hydrate, cocrystal, or prodrug thereof:

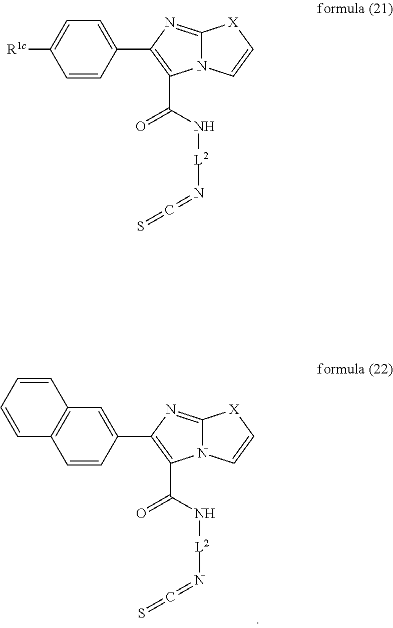

In another aspect, the disclosure relates to a compound of formula (21) or (22), or a pharmaceutically acceptable salt, solvate, hydrate, cocrystal, or prodrug thereof:

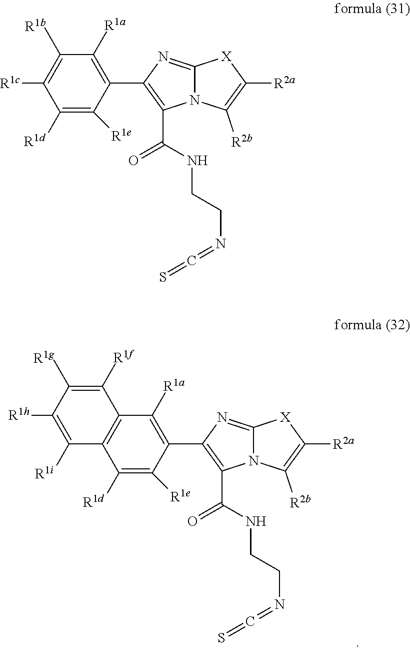

In some embodiments, R^{1c} is selected from F, Cl, and trifluoromethyl.

In another aspect, the disclosure relates to a compound of any one of formulas 1001 to 1096, or a pharmaceutically acceptable salt, solvate, hydrate, cocrystal, or prodrug thereof:

| | |
|---|---|
| | formula (21) |
| 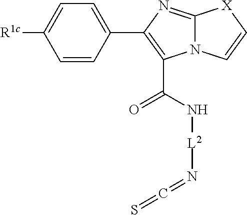 | |

| Compound | | | |
|---|---|---|---|
| # | R^{1c} | X | L² |
| 1001 | H | O | 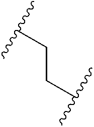 [SMILES: *CC*] |
| 1002 | —F | O | 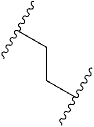 [SMILES: *CC*] |
| 1003 | —Cl | O | 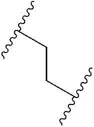 [SMILES: *CC*] |
| 1004 | —CF₃ | O | 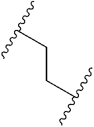 [SMILES: *CC*] |
| 1005 | H | O | 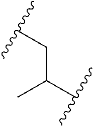 [SMILES: *CC(*)C] |
| 1006 | —F | O | 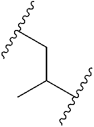 [SMILES: *CC(*)C] |
| 1007 | —Cl | O | 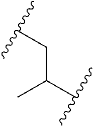 [SMILES: *CC(*)C] |
| 1008 | —CF₃ | O | 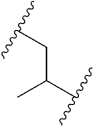 [SMILES: *CC(*)C] |
| 1009 | H | O | 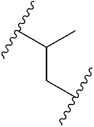 [SMILES: *CC(*)C] |
| 1010 | —F | O | 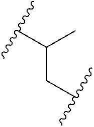 [SMILES: *CC(*)C] |
| 1011 | —Cl | O | 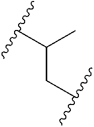 [SMILES: *CC(*)C] |
| 1012 | —CF₃ | O | 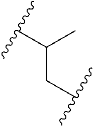 [SMILES: *CC(*)C] |
| 1013 | H | O | 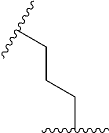 [SMILES: *CCC*] |
| 1014 | —F | O | 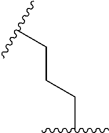 [SMILES: *CCC*] |
| 1015 | —Cl | O | 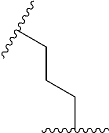 [SMILES: *CCC*] |
| 1016 | —CF₃ | O | 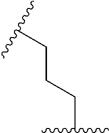 [SMILES: *CCC*] |
| 1017 | H | O | 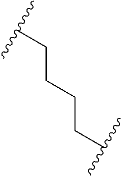 [SMILES: *CCCC*] |
| 1018 | —F | O | 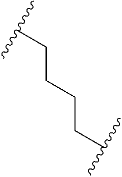 [SMILES: *CCCC*] |
| 1019 | —Cl | O | 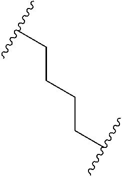 [SMILES: *CCCC*] |
| 1020 | —CF₃ | O | 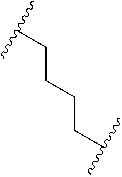 [SMILES: *CCCC*] |
| 1021 | H | O | 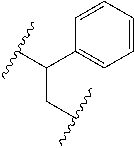 [SMILES: *CC(*)c1ccccc1] |
| 1022 | —F | O | 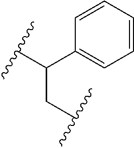 [SMILES: *CC(*)c1ccccc1] |
| 1023 | —Cl | O | 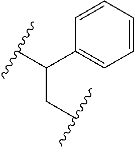 [SMILES: *CC(*)c1ccccc1] |
| 1024 | —CF₃ | O | 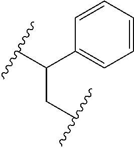 [SMILES: *CC(*)c1ccccc1] |
| 1025 | H | O | 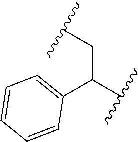 [SMILES: *CC(*)c1ccccc1] |
| 1026 | —F | O | 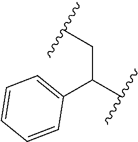 [SMILES: *CC(*)c1ccccc1] |
| 1027 | —Cl | O | 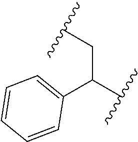 [SMILES: *CC(*)c1ccccc1] |
| 1028 | —CF₃ | O | 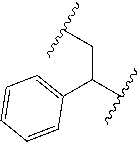 [SMILES: *CC(*)c1ccccc1] |
| 1029 | H | O | 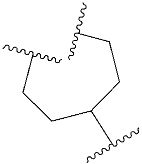 [SMILES: *CCC(*)CC*] |
| 1030 | —F | O | 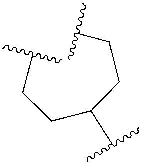 [SMILES: *CCC(*)CC*] |
| 1031 | —Cl | O | 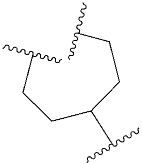 [SMILES: *CCC(*)CC*] |
| 1032 | —CF₃ | O | 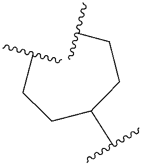 [SMILES: *CCC(*)CC*] |
| 1033 | H | O | 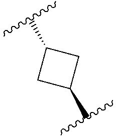 [SMILES: *[C@H]1C[C@H](*)C1] |
| 1034 | —F | O | 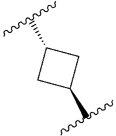 [SMILES: *[C@H]1C[C@H](*)C1] |
| 1035 | —Cl | O | 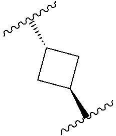 [SMILES: *[C@H]1C[C@H](*)C1] |
| 1036 | —CF₃ | O | 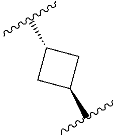 [SMILES: *[C@H]1C[C@H](*)C1] |
| 1037 | H | O | 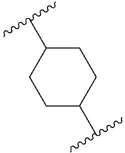 [SMILES: *C1CCC(*)CC1] |
| 1038 | —F | O | 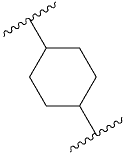 [SMILES: *C1CCC(*)CC1] |
| 1039 | —Cl | O | 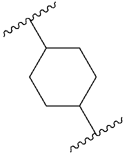 [SMILES: *C1CCC(*)CC1] |
| 1040 | —CF₃ | O | 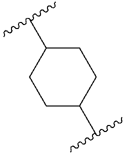 [SMILES: *C1CCC(*)CC1] |
| 1041 | H | O | 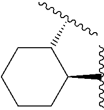 |
| 1042 | —F | O | 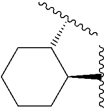 |
| 1043 | —Cl | O | 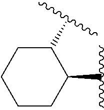 |
| 1044 | —CF₃ | O | 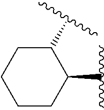 |
| 1045 | H | O | 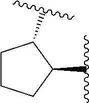 [SMILES: *[C@H]1CCC[C@@H]1*] |
| 1046 | —F | O | 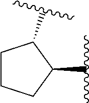 [SMILES: *[C@H]1CCC[C@@H]1*] |
| 1047 | —Cl | O | 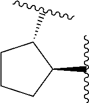 [SMILES: *[C@H]1CCC[C@@H]1*] |
| 1048 | —CF₃ | O | 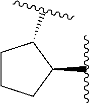 [SMILES: *[C@H]1CCC[C@@H]1*] |
| 1049 | H | S | 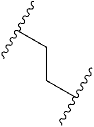 [SMILES: *CC*] |
| 1050 | —F | S | 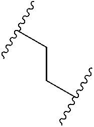 [SMILES: *CC*] |
| 1051 | —Cl | S | 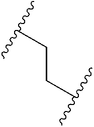 [SMILES: *CC*] |
| 1052 | —CF₃ | S | 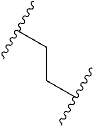 [SMILES: *CC*] |
| 1053 | H | S | 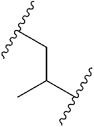 [SMILES: *CC(*)C] |
| 1054 | —F | S | 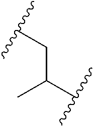 [SMILES: *CC(*)C] |
| 1055 | —Cl | S | 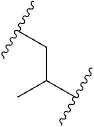 [SMILES: *CC(*)C] |
| 1056 | —CF₃ | S | 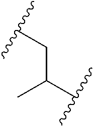 [SMILES: *CC(*)C] |
| 1057 | H | S | 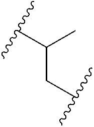 [SMILES: *CC(*)C] |
| 1058 | —F | S | 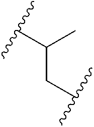 [SMILES: *CC(*)C] |
| 1059 | —Cl | S | 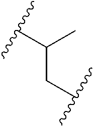 [SMILES: *CC(*)C] |
| 1060 | —CF₃ | S | 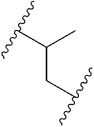 [SMILES: *CC(*)C] |
| 1061 | H | S | 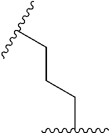 [SMILES: *CCC*] |
| 1062 | —F | S | 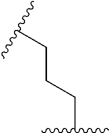 [SMILES: *CCC*] |
| 1063 | —Cl | S | 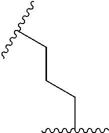 [SMILES: *CCC*] |
| 1064 | —CF₃ | S | 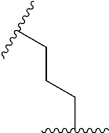 [SMILES: *CCC*] |
| 1065 | H | S | 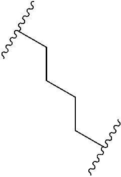 [SMILES: *CCCC*] |
| 1066 | —F | S | 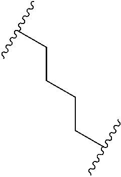 [SMILES: *CCCC*] |
| 1067 | —Cl | S | 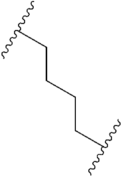 [SMILES: *CCCC*] |
| 1068 | —CF₃ | S | 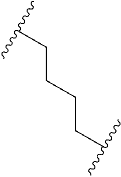 [SMILES: *CCCC*] |
| 1069 | H | S | 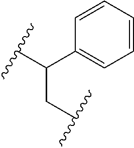 [SMILES: *CC(*)c1ccccc1] |
| 1070 | —F | S | 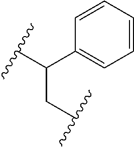 [SMILES: *CC(*)c1ccccc1] |
| 1071 | —Cl | S | 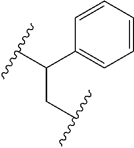 [SMILES: *CC(*)c1ccccc1] |
| 1072 | —CF₃ | S | 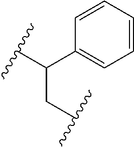 [SMILES: *CC(*)c1ccccc1] |
| 1073 | H | S | 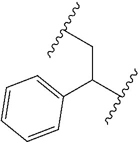 [SMILES: *CC(*)c1ccccc1] |
| 1074 | —F | S | 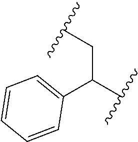 [SMILES: *CC(*)c1ccccc1] |
| 1075 | —Cl | S | 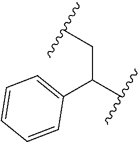 [SMILES: *CC(*)c1ccccc1] |
| 1076 | —CF₃ | S | 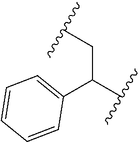 [SMILES: *CC(*)c1ccccc1] |
| 1077 | H | S | 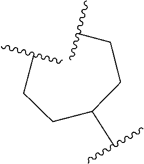 [SMILES: *CCC(*)CC*] |
| 1078 | —F | S | 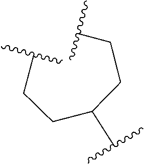 [SMILES: *CCC(*)CC*] |
| 1079 | —Cl | S | 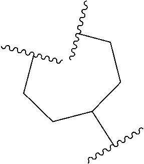 [SMILES: *CCC(*)CC*] |
| 1080 | —CF₃ | S | 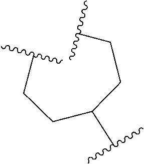 [SMILES: *CCC(*)CC*] |
| 1081 | H | S | 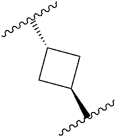 [SMILES: *[C@H]1C[C@H](*)C1] |
| 1082 | —F | S | 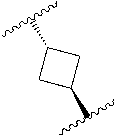 [SMILES: *[C@H]1C[C@H](*)C1] |
| 1083 | —Cl | S | 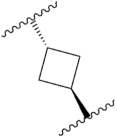 [SMILES: *[C@H]1C[C@H](*)C1] |
| 1084 | —CF₃ | S | 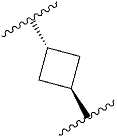 [SMILES: *[C@H]1C[C@H](*)C1] |
| 1085 | H | S | 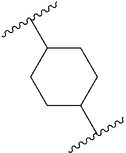 [SMILES: *C1CCC(*)CC1] |
| 1086 | —F | S | 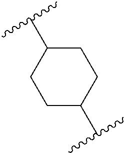 [SMILES: *C1CCC(*)CC1] |
| 1087 | —Cl | S | 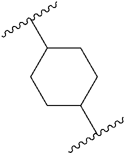 [SMILES: *C1CCC(*)CC1] |
| 1088 | —CFs | S | 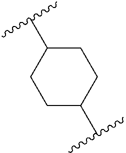 [SMILES: *C1CCC(*)CC1] |
| 1089 | H | S | 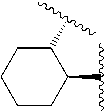 |
| 1090 | —F | S | 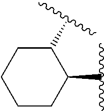 |
| 1091 | —Cl | S | 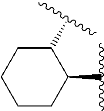 |
| 1092 | —CF₃ | S | 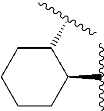 |
| 1093 | H | S | 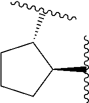 [SMILES: *[C@H]1CCC[C@@H]1*] |
| 1094 | —F | S | 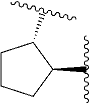 [SMILES: *[C@H]1CCC[C@@H]1*] |
| 1095 | —Cl | S | 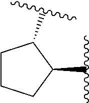 [SMILES: *[C@H]1CCC[C@@H]1*] |
| 1096 | —CF₃ | S | 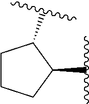 [SMILES: *[C@H]1CCC[C@@H]1*] |

In another aspect, the disclosure relates to a compound of any one of formulas 2001 to 2024, or a pharmaceutically acceptable salt, solvate, hydrate, cocrystal, or prodrug thereof:

| | | formula (22) |
|---|---|---|
| 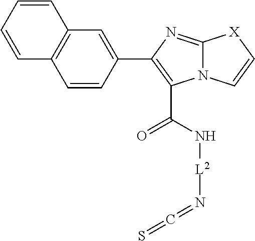 | | |
| Compound | | |
| # | X | L² |
| 2001 | O | 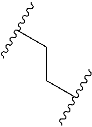 [SMILES: *CC*] |
| 2002 | O | 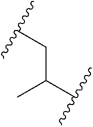 [SMILES: *CC(*)C] |
| 2003 | O | 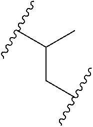 [SMILES: *CC(*)C] |
| 2004 | O | 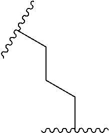 [SMILES: *CCC*] |
| 2005 | O | 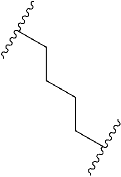 [SMILES: *CCCC*] |
| 2006 | O | 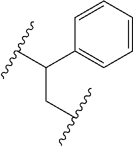 [SMILES: *CC(*)c1ccccc1] |
| 2007 | O | 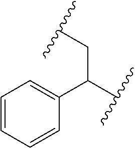 [SMILES: *CC(*)c1ccccc1] |
| 2008 | O | 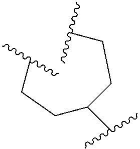 [SMILES: *CCC(*)CC*] |
| 2009 | O | 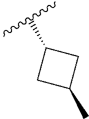 [SMILES: *[C@H]1C[C@H](C)C1] |
| 2010 | O | 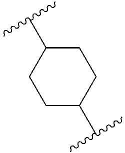 [SMILES: *C1CCC(*)CC1] |
| 2011 | O | 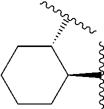 [SMILES: *[C@H]1CCCC[C@@H]1*] |
| 2012 | O | 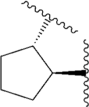 [SMILES: *[C@H]1CCC[C@@H]1*] |
| 2013 | S | 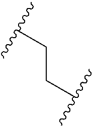 [SMILES: *CC*] |
| 2014 | S | 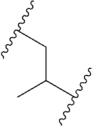 [SMILES: *CC(*)C] |
| 2015 | S | 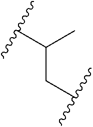 [SMILES: *CC(*)C] |
| 2016 | S | 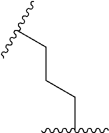 [SMILES: *CCC*] |
| 2017 | S | 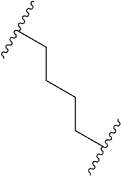 [SMILES: *CCCC*] |
| 2018 | S | 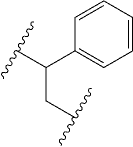 [SMILES: *CC(*)c1ccccc1] |
| 2019 | S | 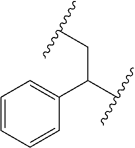 [SMILES: *CC(*)c1ccccc1] |
| 2020 | S | 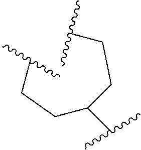 [SMILES: *CCC(*)CC*] |
| 2021 | S | 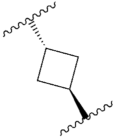 [SMILES: *[C@H]1C[C@H](*)C1] |
| 2022 | S | 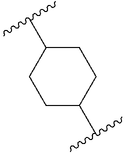 [SMILES: *C1CCC(*)CC1] |
| 2023 | S | 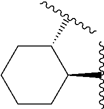 [SMILES: *[C@H]1CCCC[C@@H]1*] |
| 2024 | S | 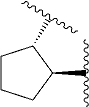 [SMILES: *[C@H]1CCC[C@@H]1*] |

In some embodiments, the compound is selected from:

| Compound # | Structure |
|---|---|
| 2013 (DL7076) | 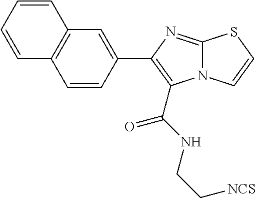 |
| 2001 (DL7009) | 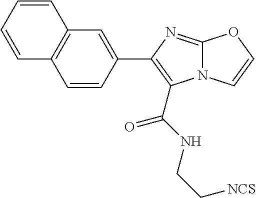 |
| 2002 (DL7092) | 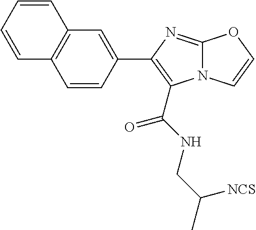 |
| 2003 (DL7091) | 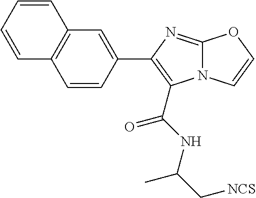 |
| 2007 (DL70552) | 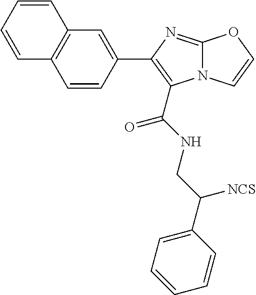 |
| 2006 (DL70562) | 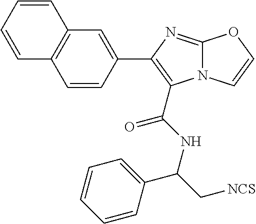 |
| 2008 (DL7077) | 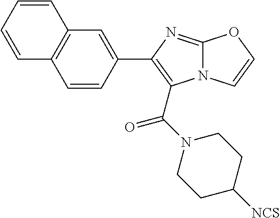 |
| 2009 (DL7102) | 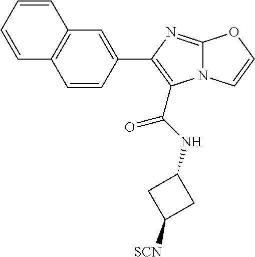 |
| 2010 (DL7086) | 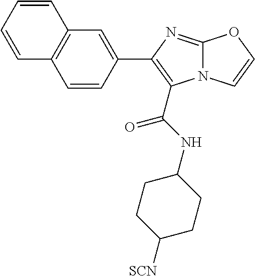 |
| 2011 (DL7096) | 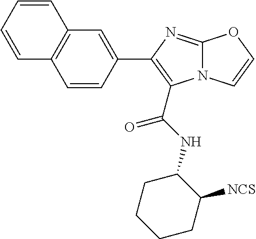 |
| 2012 (DL7097) | 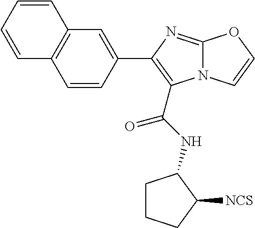 |
| 2004 (DL7087) | 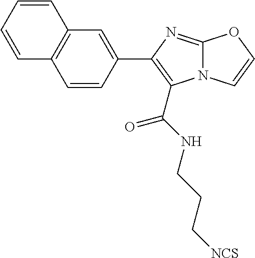 |
| 2005 (DL7101) | 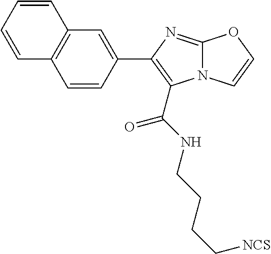 |
| 1049 (DL7134) | 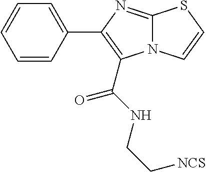 |
| 1050 (DL7135) | 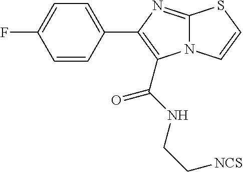 |
| 1051 (DL7128) | 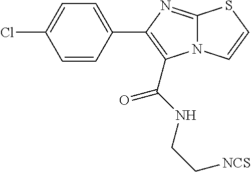 |
| 1052 (DL7127) | 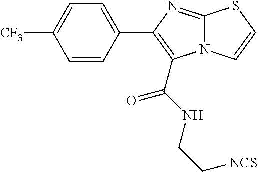 |
| 2015 (DL7139) | 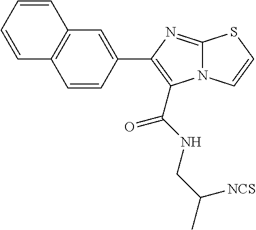 |
| 2014 (DL7140) | 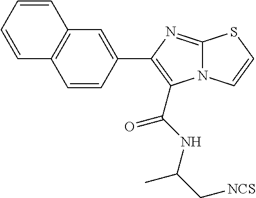 |

In some embodiments, the compound is

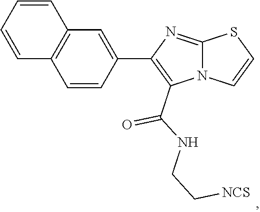

or a pharmaceutically acceptable salt, solvate, hydrate, cocrystal, or prodrug thereof.

In some embodiments, the compound is a hCAR activator.

In some embodiments, the compound is a Nrf2 activator.

In some embodiments, the compound is a dual hCAR and Nrf2 activator.

Methods of Treatment

In one aspect, the disclosure includes a method of treating a disease alleviated by activating hCAR in a patient in need thereof, the method comprising administering to the patient a therapeutically effective amount of a compound of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof.

In one aspect, the disclosure includes a method of treating a disease alleviated by activating Nrf2 in a patient in need thereof, the method comprising administering to the patient a therapeutically effective amount of a compound of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, solvate, hydrate, cocrystal, or prodrug thereof.

In one aspect, the disclosure includes a method of treating a disease alleviated by activating hCAR and Nrf2 in a patient in need thereof, comprising administering to the patient a therapeutically effective amount of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof.

Co-Culture System

In another aspect, the disclosure includes a novel multi-organ co-culture system. In some embodiments, the co-culture system comprises at least one hepatocyte, at least one cancer cell, and at least one cardiomyocyte. In a non-limiting example, the co-culture system can be used to measure efficacy in vitro while also mimicking in vivo conditions of the human body, allowing studies of hepatic metabolism, drug-drug interaction, and extrahepatic effects such as, anti-cancer effect and side toxicities.

In some embodiments, the hepatocyte is a human primary hepatocyte (HPH). In some embodiments, the cancer cell is a breast cancer cell. In some embodiments, the breast cancer cell is a triple negative breast cancer cell. In some embodiments, the cardiomyocyte is a H9c2 cardiomyocyte.

Pharmaceutical Compositions and Routes of Administration

In one embodiment, the disclosure provides a pharmaceutical composition for use in the treatment of the diseases and conditions described herein. In a preferred embodiment, the disclosure provides pharmaceutical compositions, including those described below, for use in the treatment of hematological malignancy. In one embodiment, the disclosure provides a pharmaceutical composition comprising a compound of formula (I). In one embodiment, the disclosure provides a pharmaceutical composition comprising a compound of formula (1). In one embodiment, the disclosure provides a pharmaceutical composition comprising a compound of formula (2). In one embodiment, the disclosure provides a pharmaceutical composition comprising a compound of formula (11). In one embodiment, the disclosure provides a pharmaceutical composition comprising a compound of formula (12). In one embodiment, the disclosure provides a pharmaceutical composition comprising a compound of formula (21). In one embodiment, the disclosure provides a pharmaceutical composition comprising a compound of formula (22). In one embodiment, the disclosure provides a pharmaceutical composition comprising a compound of formula (31). In one embodiment, the disclosure provides a pharmaceutical composition comprising a compound of formula (32). In one embodiment, the disclosure provides a pharmaceutical composition comprising a compound of any one of formulas 1001 to 1096. In one embodiment, the disclosure provides a pharmaceutical composition comprising a compound of any one of formulas 2001 to 2024. In one embodiment, the disclosure provides a pharmaceutical composition comprising a compound of any one of formulas 1049, 1050, 1051, 1052, 2001, 2002, 2003, 2004, 2005, 2006, 2007, 2008, 2009, 2010, 2011, 2012, 2013, 2014, and 2015.

Any compound disclosed herein, for example a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, may be administered to a subject by itself, or in the form of a pharmaceutical composition. Pharmaceutical compositions comprising the compounds of the disclosure may be manufactured by means of conventional mixing, dissolving, granulating, dragee-making, levigating, emulsifying, encapsulating, entrapping or lyophilizing processes. Pharmaceutical compositions may be formulated in conventional manner using one or more physiological acceptable carriers, diluents, excipients, or auxiliaries which facilitate processing of the compounds into preparations which can be used pharmaceutically. A specific formulation method will be dependent upon the route of administration chosen.

The pharmaceutical compositions are typically formulated to provide a therapeutically effective amount of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, or a fragment, derivative, conjugate, variant, radioisotope-labeled complex, or biosimilar thereof, or pharmaceutically acceptable salts, prodrugs, solvates, or hydrates thereof, as the active ingredients. Where desired, the pharmaceutical compositions contain a pharmaceutically acceptable salt and/or coordination complex of one or more of the active ingredients.

Where desired, other active pharmaceutical ingredient(s) may be mixed into a preparation or two or more components of the combination may be formulated into separate preparations for use in combination separately or at the same time. A kit containing the components of the combination, formulated into separate preparations for said use, is also provided by the disclosure.

Where desired, other active pharmaceutical ingredient(s) may be mixed into a preparation or two or more components of the combination may be formulated into separate preparations for use in combination separately or at the same time. A kit containing the components of the combination, formulated into separate preparations for said use, is also provided by the disclosure.

In some embodiments, the concentration of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, provided in a pharmaceutical composition of the disclosure, is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v of the pharmaceutical composition.

In some embodiments, the concentration of a compound of formula (I), formula (1), formula (2), formula (11), formula (12), formula (21), formula (22), formula (31), formula (32), formulas 1001 to 1096, or formulas 2001 to 2024, provided in a pharmaceutical composition of the disclosure, is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v of the pharmaceutical composition.

In some embodiments, the concentration of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, provided in a pharmaceutical composition of the disclosure, is independently in the range from about 0.0001% to about 50%, from about 0.001% to about 40%, from about 0.01% to about 30%, from about 0.02% to about 29%, from about 0.03% to about 28%, from about 0.04% to about 27%, from about 0.05% to about 26%, from about 0.06% to about 25%, from about 0.07% to about 24%, from about 0.08% to about 23%, from about 0.09% to about 22%, from about 0.1% to about 21%, from about 0.2% to about 20%, from about 0.3% to about 19%, from about 0.4% to about 18%, from about 0.5% to about 17%, from about 0.6% to about 16%, from about 0.7% to about 15%, from about 0.8% to about 14%, from about 0.9% to about 12%, or from about 1% to about 10% w/w, w/v, or v/v of the pharmaceutical composition.

In some embodiments, the concentration of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, provided in a pharmaceutical composition of the disclosure, is independently in the range from about 0.001% to about 10%, from about 0.01% to about 5%, from about 0.02% to about 4.5%, from about 0.03% to about 4%, from about 0.04% to about 3.5%, from about 0.05% to about 3%, from about 0.06% to about 2.5%, from about 0.07% to about 2%, from about 0.08% to about 1.5%, from about 0.09% to about 1%, from about 0.1% to about 0.9% w/w, w/v, or v/v of the pharmaceutical composition.

In some embodiments, the amount or dose of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, provided in a pharmaceutical composition of the disclosure, is independently equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the amount or dose of a compound of formula (I), formula (1), formula (2), formula (11), formula (12), formula (21), formula (22), formula (31), formula (32), formulas 1001 to 1096, or formulas 2001 to 2024, or pharmaceutically acceptable salt thereof, provided in a pharmaceutical composition of the disclosure, is independently more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5, about 3 g, about 3.5, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

Each of the compounds of formula (I), formula (1), formula (2), formula (11), formula (12), formula (21), formula (22), formula (31), formula (32), formulas 1001 to 1096, or formulas 2001 to 2024, or pharmaceutically acceptable salt thereof is effective over a wide dosage range. For example, in the treatment of adult humans, dosages independently ranging from about 0.01 to about 1000 mg, from about 0.5 to about 100 mg, from about 1 to about 50 mg per day, and from about 5 to about 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Cyclophosphamide (CPA), an alkylating prodrug, has been used extensively in the treatment of various types of cancers. CPA is a mainstay of numerous drug combinations, most importantly the CHOP regimen. CHOP is named after the initials of the drugs used which are CPA, doxorubicin, Oncovin (vincristine), and prednisone. DOX, an anti-neoplastic agent, is widely used and considered highly effective in combination with CPA. CHOP consists of CAP, an alkylating agent which damages DNA by binding to it and causing the formation of cross-links hydroxydaunorubicin (also called doxorubicin or adriamycin), an intercalating agent which damages DNA by inserting itself between DNA bases Oncovin (vincristine), which prevents cells from duplicating by binding to the protein tubulin prednisone or prednisolone, which are corticosteroids. CHOP is the first-line chemotherapy for non-Hodgkin's lymphoma and a number of other hematologic malignancies. A unit dose of CHOP consists of 750-1200 mg/m² of CPA, 50-75 mg mg/m² of doxorubicin, 1.4 mg/m² (maximum 2 mg) of vincristine and 40-100 mg/m² of prednisone.

In one aspect, the disclosure relates generally to novel compounds/agents which can enhance the therapeutic efficacy of cyclophosphamide-based and doxorubicin-based chemotherapy, pharmaceutical compositions comprising such novel compounds/agents and methods of use thereof.

In another aspect, the disclosure provides combination therapies for the treatment of cancer. In particular, the disclosure provides combination therapies of known CYP2B6 substrate anticancer agent e.g. cyclophosphamide and/or docyrubicin for treating cancer. In one aspect the disclosure provides compositions and methods for treating cancer with a dual human constitutively active receptor (hCAR) and erythroid 2-related factor 2 (Nrf2) activator that induces the expression of CYP2B6 and reduces DOX-induced cardiotoxicity, e.g., DL7076 in combination with a cyclophosphamide-based and doxorubicin-based chemotherapy. Such combination provides sensitization effects in the treatment of cancers and a protective effect against DOX-induced cardiotoxicity and particularly treatment of hematologic malignancies.

In another aspect the disclosure provides novel compounds that are dual hCAR and Nrf2 activators, such as DL7076, acting as a selective hCAR activator and reducing DOX-induced cardiotoxicity, that potentiates the efficacy: toxicity ratio of CPA-based and DOX-based chemotherapy to facilitate cyclophosphamide (CPA)-based and DOX-based chemotherapy for hematologic malignancies. DL5016 induced hCAR activation potentiates the benefits of CPA, without altering its side effect profile.

In another aspect the disclosure provides the methods for synthesis of these new molecules, including DL7076. In a further aspect the disclosure provides biological effects of compounds, e.g., DL7076 on the selective induction of CYP2B6 over CYP3A4 at both mRNA and protein levels, as well as a reduction in DOX-induced cardiotoxicity.

A further embodiment of the pharmaceutical composition comprises the dual hCAR and Nrf2 activators described herein, for example a compound of formula (I), formula (1), formula (2), formula (11), formula (12), formula (21), formula (22), formula (31), formula (32), formulas 1001 to 1096, or formulas 2001 to 2024, a pharmaceutically acceptable salt, solvate, hydrate, cocrystal, or prodrug thereof, at least one oxazaphosphorine class of antineoplastic agent based chemotherapy, at least one anthracycline class of antineoplastic agent based chemotherapy, and a physiologically compatible carrier. Non-limiting examples of oxazaphosphorine antineoplastic agents include cyclophosphamide, ifosfamide and trofosfamide. Non-limiting examples of anthracycline antineoplastic agents include doxorubicin, epirubicin, daunorubicine, and mitoxantrone.

In a further embodiment, the disclosure provides pharmaceutical compositions comprising the combination of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, with cyclophosphamide and doxorubicin for the treatment of non-Hodgkin lymphoma, chronic lymphocytic leukemia, triple negative breast cancers, and other solid tumors.

In one embodiment, the disclosure provides pharmaceutical compositions including the combination of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, with cyclophosphamide and doxorubicin for the treatment of hematologic malignancies. In one embodiment, the disclosure provides pharmaceutical compositions including the combination of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, with cyclophosphamide-based and docyrubicin-based chemotherapy regimen (e.g. CHOP) for the treatment of non-Hodgkin lymphoma, chronic lymphocytic leukemia, triple negative breast cancers, and other solid tumors. In one embodiment, the disclosure provides the combination of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof, a CPA based chemotherapy, and a DOX-based chemotherapy. In one embodiment, the disclosure provides the combination of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof and the CHOP treatment regimen.

Described below are non-limiting pharmaceutical compositions and methods for preparing the same.

Pharmaceutical Compositions for Oral Administration

In preferred embodiments, the disclosure provides a pharmaceutical composition for oral administration containing one or more of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, and a pharmaceutical excipient suitable for administration. In other embodiments, the disclosure provides a pharmaceutical composition for oral administration containing one or more of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, a pharmaceutical excipient suitable for administration, and one or more additional active pharmaceutical ingredient.

In some embodiments, the pharmaceutical composition may be a solid pharmaceutical composition suitable for oral consumption. In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the compounds of formula (I), formula (1), formula (2), formula (11), formula (12), formula (21), formula (22), formula (31), formula (32), formulas 1001 to 1096, or formulas 2001 to 2024 used as active ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, 0-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of about 10%, about 25%, about 50%, about 100%, or up to about 200% by weight, based on the combined weight of the drug, and other excipients. If desired, very small amounts of solubilizer may also be used, such as about 5%, about 2%, about 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not be limited to, sodium, potassium, lithium, magnesium, calcium, and/or ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

Pharmaceutical Compositions for Injection

In preferred embodiments, the disclosure provides a pharmaceutical composition for injection of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, and a pharmaceutical excipient suitable for injection. Components and amounts of agents in the compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an hCAR activator described herein, for example of a compound of formula (I), formula (1), formula (2), formula (11), formula (12), formula (21), formula (22), formula (31), formula (32), formulas 1001 to 1096, or formulas 2001 to 2024, according to the disclosure, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical Delivery

In some embodiments, the disclosure provides a pharmaceutical composition for transdermal delivery containing a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of the compounds of the disclosure, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

Pharmaceutical Compositions for Inhalation

In some embodiments, the disclosure provides a pharmaceutical composition for inhalation or insufflation delivery containing a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, or pharmaceutical compositions of these compounds, can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compounds or compositions thereof can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include each of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, or pharmaceutical compositions thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient.

Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

In some embodiments, the disclosure provides a kit comprising a composition comprising a therapeutically effective amount of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, according to the disclosure, or a fragment, derivative, conjugate, variant, radioisotope-labeled complex, biosimilar, pharmaceutically acceptable salt, solvate, hydrate, cocrystal, or prodrug thereof. These compositions are typically pharmaceutical compositions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery system may be employed. Liposomes and emulsions are well known examples of delivery vehicles that may be used to deliver the compounds of the disclosure. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. A variety of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed. As the compounds of the disclosure may contain charged side chains or termini, they may be included in any of the above-described formulations as the free acids or bases or as pharmaceutically acceptable salts. Pharmaceutical salts tend to be more soluble in aqueous and other protic solvents than are the corresponding free acid or base forms.

The methods of the present disclosure will normally include medical follow-up to determine the therapeutic or prophylactic effect brought about in the patient undergoing treatment with the compound(s) and/or composition(s) described herein. Efficacy of the methods may be assessed on the basis of tumor regression, e.g., reducing the size and/or number of neoplasms, inhibition of tumor metastasis, decrease in a serological marker of disease, or other indicator of an inhibitory or remedial effect.

Dosages and Dosing Regimens

The amount of the compound of formula (I), formula (1), formula (2), formula (11), formula (12), formula (21), formula (22), formula (31), formula (32), formulas 1001 to 1096, or formulas 2001 to 2024, to be administered, will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day. The dosage of the compound of formula (I), formula (1), formula (2), formula (11), formula (12), formula (21), formula (22), formula (31), formula (32), formulas 1001 to 1096, or formulas 2001 to 2024, may be provided in units of mg/kg of body mass, or in mg/m² of body surface area.

In some embodiments, the compound of formula (I), formula (1), formula (2), formula (11), formula (12), formula (21), formula (22), formula (31), formula (32), formulas 1001 to 1096, or formulas 2001 to 2024, is administered in a single dose. Such administration may be by injection, e.g., intravenous injection, in order to introduce the hCAR activator quickly. However, other routes, including the oral route, may be used as appropriate. A single dose of the hCAR activator may also be used for treatment of an acute condition.

In some embodiments, the compound of formula (I), formula (1), formula (2), formula (11), formula (12), formula (21), formula (22), formula (31), formula (32), formulas 1001 to 1096, or formulas 2001 to 2024, is administered in multiple doses. In a preferred embodiment, the hCAR activator is administered in multiple doses. Dosing may be once, twice, three times, four times, five times, six times, or more than six times per day. Dosing may be once a month, once every two weeks, once a week, or once every other day. In other embodiments, the compound of formula (I), formula (1), formula (2), formula (11), formula (12), formula (21), formula (22), formula (31), formula (32), formulas 1001 to 1096, or formulas 2001 to 2024, is administered about once per day to about 6 times per day. In some embodiments, the compound of formula (I), formula (1), formula (2), formula (11), formula (12), formula (21), formula (22), formula (31), formula (32), formulas 1001 to 1096, or formulas 2001 to 2024, is administered once daily, while in other embodiments, the compound of formula (I), formula (1), formula (2), formula (11), formula (12), formula (21), formula (22), formula (31), formula (32), formulas 1001 to 1096, or formulas 2001 to 2024, is administered twice daily, and in other embodiments the compound of formula (I), formula (1), formula (2), formula (11), formula (12), formula (21), formula (22), formula (31), formula (32), formulas 1001 to 1096, or formulas 2001 to 2024, is administered three times daily.

Administration of the active pharmaceutical ingredients of the disclosure may continue as long as necessary. In some embodiments, the compound of formula (I), formula (1), formula (2), formula (11), formula (12), formula (21), formula (22), formula (31), formula (32), formulas 1001 to 1096, or formulas 2001 to 2024, is administered for more than 1, 2, 3, 4, 5, 6, 7, 14, or 28 days. In some embodiments, the compound of formula (I), formula (1), formula (2), formula (11), formula (12), formula (21), formula (22), formula (31), formula (32), formulas 1001 to 1096, or formulas 2001 to 2024, is administered for less than 28, 14, 7, 6, 5, 4, 3, 2, or 1 day. In some embodiments, the compound of formula (I), formula (1), formula (2), formula (11), formula (12), formula (21), formula (22), formula (31), formula (32), formulas 1001 to 1096, or formulas 2001 to 2024, is administered chronically on an ongoing basis, e.g., for the treatment of chronic effects. In another embodiment the administration of the hCAR activator continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of a compound of formula (I), formula (1), formula (2), formula (11), formula (12), formula (21), formula (22), formula (31), formula (32), formulas 1001 to 1096, or formulas 2001 to 2024, is in the range of about 1 mg to about 50 mg, about 5 mg to about 45 mg, about 10 mg to about 40 mg, about 15 mg to about 35 mg, about 20 mg to about 30 mg, about 23 mg to about 28 mg, about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, about 198 to about 202 mg, or about 198 to about 207 mg.

In some embodiments, an effective dosage of a compound of formula (I), formula (1), formula (2), formula (11), formula (12), formula (21), formula (22), formula (31), formula (32), formulas 1001 to 1096, or formulas 2001 to 2024, is about 25 mg, about 50 mg, about 75 mg, about 100 mg, about 125 mg, about 150 mg, about 175 mg, about 200 mg, about 225 mg, or about 250 mg.

In some embodiments, an effective dosage of a compound of formula (I), formula (1), formula (2), formula (11), formula (12), formula (21), formula (22), formula (31), formula (32), formulas 1001 to 1096, or formulas 2001 to 2024, is in the range of about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of a compound of formula (I), formula (1), formula (2), formula (11), formula (12), formula (21), formula (22), formula (31), formula (32), formulas 1001 to 1096, or formulas 2001 to 2024, is about 0.35 mg/kg, about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of a compound of formula (I), formula (1), formula (2), formula (11), formula (12), formula (21), formula (22), formula (31), formula (32), formulas 1001 to 1096, or formulas 2001 to 2024, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

Methods of Treating Hematological Malignancies and Solid Tumors

In some embodiments, the disclosure relates to a method of treating a disease alleviated by activating hCAR enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating hCAR enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating hCAR enzyme in a patient in need thereof, including administering to the patient a therapeutically effective amount of one or more compounds of Formulas (I) and (II), or a pharmaceutically acceptable salt, solvate, hydrate, cocrystal, or prodrug thereof.

In a preferred embodiment, the patient or subject is a mammal, such as a human. In an embodiment, the patient or subject is a human. In an embodiment, the patient or subject is a companion animal. In an embodiment, the patient or subject is a canine, feline, or equine.

In some embodiments, the disclosure relates to a method of treating a disease alleviated by activating hCAR enzyme, in a patient in need thereof, including administering to the patient dosage unit form including a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof. In some embodiments, the dosage unit form includes a physiologically compatible carrier medium.

In some embodiments, the disclosure relates to a method of treating a cancer alleviated by activating hCAR enzyme, in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating Nrf2 enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating Nrf2 enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating Nrf2 enzyme in a patient in need thereof, including administering to the patient a therapeutically effective amount of one or more compounds of Formulas (I) and (II), or a pharmaceutically acceptable salt, solvate, hydrate, cocrystal, or prodrug thereof.

In some embodiments, the disclosure relates to a method of treating a disease alleviated by activating Nrf2 enzyme, in a patient in need thereof, including administering to the patient dosage unit form including a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof. In some embodiments, the dosage unit form includes a physiologically compatible carrier medium.

In some embodiments, the disclosure relates to a method of treating a cancer alleviated by activating Nrf2 enzyme, in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating hCAR enzyme and Nrf2 enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating hCAR enzyme and Nrf2 enzyme in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, the disclosure relates to a method of treating a disease alleviated by activating hCAR enzyme and Nrf2 enzyme in a patient in need thereof, including administering to the patient a therapeutically effective amount of one or more compounds of Formulas (I) and (II), or a pharmaceutically acceptable salt, solvate, hydrate, cocrystal, or prodrug thereof.

In some embodiments, the disclosure relates to a method of treating a disease alleviated by activating hCAR enzyme and Nrf2 enzyme, in a patient in need thereof, including administering to the patient dosage unit form including a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof. In some embodiments, the dosage unit form includes a physiologically compatible carrier medium.

In some embodiments, the disclosure relates to a method of treating a cancer alleviated by activating hCAR enzyme and Nrf2 enzyme, in a patient in need thereof, including administering to the patient a therapeutically effective amount of one or more compounds of formula (I), formula (1), formula (2), formula (11), formula (12), formula (21), formula (22), formula (31), formula (32), formulas 1001 to 1096, and/or formulas 2001 to 2024, or a pharmaceutically acceptable salt, solvate, hydrate, cocrystal, or prodrug thereof.

In some embodiments, CYP2B6 is selectively induced over CYP3A4.

In some embodiments, the method further comprising administering to the patient a therapeutically effective amount of cyclophosphamide (CPA) and doxorubicin (DOX), or a pharmaceutically acceptable salt, solvate, hydrate, cocrystal, or prodrug thereof.

In some embodiments, CPA and DOX are administered as part of the CHOP regimen (CPA, doxorubicin, vincristine, and prednisone).

In some embodiments, co-administration of a compound of any one of claims 1-25, or a pharmaceutically acceptable salt, solvate, hydrate, cocrystal, or prodrug thereof, CPA, and DOX, promotes the formation of therapeutically active CPA metabolite 4-OH-CPA and decreased cleaved caspase-3 expression.

In some embodiments, the compound, or pharmaceutically acceptable salt, solvate, hydrate, cocrystal, or prodrug thereof, is administered in a dosage unit form.

In some embodiments, the dosage unit form comprises a physiologically compatible carrier medium.

In some embodiments, the disease is cancer.

In some embodiments, the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the cancer is a triple negative breast cancer (TNBC).

In some embodiments, co-administration of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof, CPA, and DOX enhances the efficacy of CPA and DOX and reduces cardiotoxicity than administering CPA and DOX alone.

In some embodiments, co-administration of a compound of formula (I), formula (1), formula (2), formula (11), formula (12), formula (21), formula (22), formula (31), formula (32), formulas 1001 to 1096, or formulas 2001 to 2024, or a pharmaceutically acceptable salt, solvate, hydrate, cocrystal, or prodrug thereof, CPA, and DOX permits the administration of a lower dose of CPA and/or DOX than when CPA and DOX are administered alone.

In some embodiments, the patient or subject is a mammal, such as a human. In an embodiment, the patient or subject is a human. In an embodiment, the patient or subject is a companion animal. In an embodiment, the patient or subject is a canine, feline, or equine.

In some embodiments, the hematological malignancies can be breast cancer, non-Hodgkin lymphoma, and chronic lymphocytic leukemia.

Combinations of hCAR and Nrf2 Dual Activators with CPA Based and DOX Based Treatment Regimen

In one aspect, the hCAR and Nrf2 dual activators described herein can also be co-administered with additional chemotherapeutic active pharmaceutical ingredients, for example doxorubicin, vincristine, and prednisone. In some embodiments, the disclosure provides a method of treating a hematological malignancy or a solid tumor cancer in a human including the step of administering to said human a compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, and further including the step of administering a therapeutically-effective amount of gemcitabine, or a pharmaceutically acceptable salt, prodrug, cocrystal, solvate or hydrate thereof.

In some embodiments, the disclosure provides a method of treating a hematological malignancy or a solid tumor cancer in a human including the step of administering to said human a compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, prodrug, cocrystal, solvate or hydrate thereof, and further including the step of administering a therapeutically-effective amount of gemcitabine, or a pharmaceutically acceptable salt, prodrug, cocrystal, solvate or hydrate thereof. In an embodiment, the solid tumor cancer in any of the foregoing embodiments is pancreatic cancer.

As used herein, the term “combination” or “pharmaceutical combination” refers to the combined administration of therapeutic agents, for example a compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, and an anticancer agent. Combinations of the disclosure include a compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, and at least one anticancer agent, for example CPA and/or DOX. The term “synergistic” or “synergistic effect” or “synergism” as used herein, generally refers to an effect such that the one or more effects of the combination of compositions is greater than the one or more effects of each component al one, or they can be greater than the sum of the one or more effects of each component alone. The synergistic effect can be greater than about 10%, 20%, 30%, 50%, 75%, 100%, 110%, 120%, 150%, 200%, 250%, 350%, or 500%, or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

In any of the foregoing embodiments, the chemotherapeutic active pharmaceutical ingredient, for example CPA and/or DOX, or combinations thereof, may be administered before, concurrently, or after administration of the compound of formula (I), formula (1), formula (2), formula (11), formula (12), formula (21), formula (22), formula (31), formula (32), formulas 1001 to 1096, and/or formulas 2001 to 2024, or a pharmaceutically acceptable salt, described herein.

While preferred embodiments of the disclosure are shown and described herein, such embodiments are provided by way of example only and are not intended to otherwise limit the scope of the disclosure, and various alternatives to the described embodiments of the disclosure may be employed in practice.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: CAR and Nrf2 Dual Activator DL7076: A Novel Improvement to Cyclophosphamide and Doxorubicin-Based Treatment of TNBC

As described herein, DL7076 has been identified as a CAR-Nrf2 dual activator, which activates Nrf2 in cardiomyocytes and CAR in human primary hepatocytes. Coadministration of DL7076 demonstrates enhanced cytotoxicity of the CPA/DOX combination in TNBC, while alleviating cytotoxicity in cardiomyocytes.

DL7076 concentration-dependently induced the expression of CYP2B6 in human primary hepatocytes and HO-1, a Nrf2 target gene, in cardiomyocytes and human iPSC-derived cardiomyocytes. DOX-induced cytotoxicity in H9c2 cells and iPSC-derived cardiomyocytes was efficiently reversed by co-administration of DL7076, which was not seen in TNBC cells.

DL7076 exhibits selective activation of Nrf2 in H9c2 over TNBC cells, which is further supplemented by DL7076 reduction in reactive oxygen species. Using a hepatocyte/cardiomyocytes/TNBC co-culture model, it was demonstrated that co-administration of DL7076 enhanced the anticancer activity of CPA and reduced the cardiotoxicity. Taken together, these findings show that DL7076 is a potent dual activator of hCAR/Nrf2, which shows tissue specific induction of CYP2B6 in primary hepatocytes and activation of Nrf2 in the off-target cardiomyocytes showing potential improvement of CPA/DOX efficacy: toxicity ratio in CPA/DOX containing regimens for the treatment of TNBC.

### Chemical Synthesis Methods

The compounds of this disclosure may be made by various methods known in the art. Such methods include those of the following reaction schemes, as well as the methods specifically exemplified below. Modifications of such methods and schemes that involve techniques commonly practiced in the art of organic synthesis may also be used. The variable numbering and structure numbering shown in the synthetic schemes are distinct from, and should not be confused with, the variables or structure numbering in the claims or other parts of the specification. The variables in the schemes are meant only to illustrate how to make certain of the compounds of this disclosure. General routes suitable to prepare chemical species exemplified herein are shown in the schemes 1-4 below. The reagents and starting materials are commercially available, or readily synthesized by well-known techniques by one of ordinary skill in the art. All preparative methods disclosed herein are contemplated to be implemented on any scale, including milligram, gram, multigram, kilogram, multikilogram or commercial industrial scale.

Abbreviations

The symbols and conventions used in the reaction schemes and preparative examples set forth below are consistent with those used in the contemporary scientific literature, for example, the journal of the American Chemical Society or the Journal of Biological Chemistry. Specifically, the following abbreviations may be used in the examples and throughout the specification:
g (grams);

DCM (dichloromethane);

EtOAc (ethylacetate);

NaHCO₃ (sodium bicarbonate);

Na₂SO₄ (sodium sulfate);

POCl₃ (phosphoryl chloride);

CSCl₂ (thiophosgene)

EtOH (ethanol);

DMF (N,N-dimethylformamide);

CHCl₃ (chloroform);

AcOH (acetic acid);

TFA (trifluoroacetic acid);

TEA (triethylamine);

H₂O (water);

THF (tetrahydrofuran);

HATU (1-[Bis(dimethylamino)methylene]-1H-1,2,3-triazolo[4,5-b]pyridinium 3-oxide hexafluorophosphate)

DMAP (4-Dimethylaminopyridine);

CH₃OH (methanol);

DIPEA (N,N-diisopropylethylamine);

Boc (tert-butyloxycarbonyl);

K₂CO₃ (potassium carbonate);

EA/HE (ethyl acetate/hexane);

h (hour);

mg (milligrams);

mL (milliliters);

mmol (millimole);

equiv. (equivalent);

Ac (acetyl);

Et (ethyl);

TLC (thin-layer chromatography);

HPLC (high-performance liquid chromatography);

NMR spectra (nuclear magnetic resonance spectroscopy);

¹H NMR (proton nuclear magnetic resonance spectroscopy);

¹³C NMR (carbon 13 nuclear magnetic resonance spectroscopy);

UV spectra (ultra violet spectra);

HRMS (high-resolution mass spectra);

ESI (electron spray ionization);

APCI (atmospheric pressure chemical ionization);

Vilsmeier reagent (chloromethylene)dimethyliminium chloride;

Arnold's reagent, dimethylchloroformiminium chloride).

Unless otherwise indicated in the examples, all temperature is expressed in Centigrade (C). All reactions were conducted under an inert atmosphere at ambient temperature unless otherwise noted. Reagents employed without synthetic details are commercially available or made according to literature procedures.

### Materials and Methods:

### General Procedures

### General Information

All reagents were purchased without further purification unless otherwise noted. Reactions were monitored using thin-layer chromatography (TLC) on commercial silica-gel plates (GF254). Visualization of the developed plates was performed under UV light (254 nm). Flash column chromatography was performed on silica gel (200-300 mesh). ¹H and ¹³C NMR spectra were recorded on a Varian INOVA 400 MHz NMR spectrometer at 25° C. Chemical shifts (δ) are reported in ppm and referenced to the CDCl₃ residual peak (δ 7.26) or DMSO-d6 residual peak (δ 2.50) for ¹H NMR. Chemical shifts of ¹³C NMR are reported relative to CDCl₃ (δ 77.0) or DMSO-d6 (δ 39.5). The following abbreviations were used to describe peak-splitting patterns when appropriate: br s, broad singlet; s, singlet; d, doublet; t, triplet; q, quartet; and m, multiplet. Coupling constants, J, were reported in hertz units (Hz). High-resolution mass spectra (HRMS) were obtained on a JEOL AccuTOF with ESI and APCI ion sources and coupled to an Agilent 1100 HPLC system.

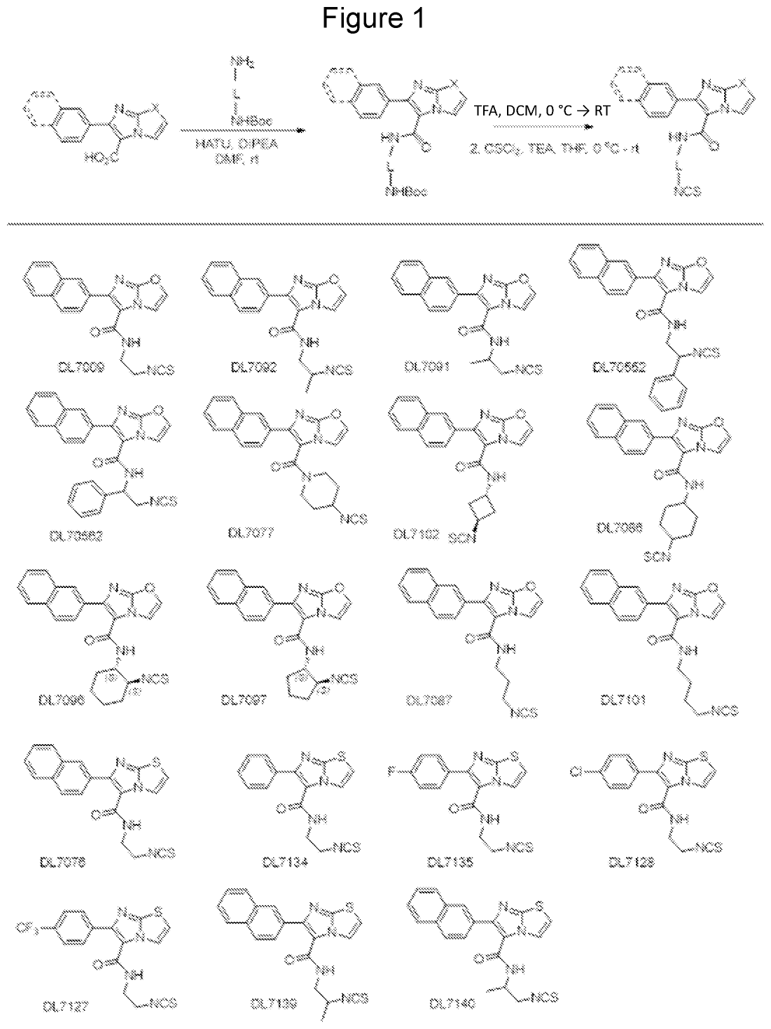

A general method for synthesis of compounds of Formula (I), including exemplary compounds prepared using same, is shown in FIG. 1.

### General Synthesis Procedure

To a solution of substituted imidazo[2,1-b]oxazole-5-carboxylic acid or imidazo[2,1-b]thiazole-5-carboxylic acid (1 mmol, which were synthesized according to ACS Med. Chem. Lett. 2019, 10, 1039-1044 and Euro. J. Med. Chem. 2019, 179, 84-99, which is incorporated by reference herein in its entirety) in DMF (5 mL) was added HATU (1.2 mmol) and DIPEA (3 mmol), followed by the corresponding mono Boc protected diamine (1 mmol). Upon completion of the reaction (followed by TLC), the reaction mixture was partitioned between NaCl (25 mL) and EtOAc (25 mL). The aqueous layer was extracted by EtOAc (25 mL×2). The combined organic layers were dried over Na₂SO₄, and concentrated. The crude material was purified by flash chromatography to give amide compound with Boc protected amine.

The Boc protected amine (0.5 mmol) was taken up in Trifluoroacetic acid (1 ml) and dichloromethane (1 ml) mixture at 0° C. and allowed to stir at room temperature for 2 h. The reaction mixture was diluted with EtOAc and concentrated under reduced pressure to afford the TFA salt of amine. To a solution of the TFA salt of amine (0.5 mmol) in 5 mL of THF was added dropwise triethylamine (1.5 mmol) at 0° C. After stirring for 5 min, thiophosgene (0.5 mmol) was added dropwise at 0° C. The resulting solution was slowly warmed to room temperature, stirred for 1.5 h and then cooled to 0° C. The reaction mixture was partitioned between H₂O (25 mL) and EtOAc (25 mL). The aqueous layer was extracted by EtOAc (25 mL×2). The combined organic layers were dried over Na₂SO₄, and concentrated. The crude material was purified by flash chromatography to give final compounds.

### Characterization of the Main Intermediates and Final Compounds

### Tert-butyl (2-(6-(naphthalen-2-yl)imidazo[2,1-b]oxazole-5-carboxamido)ethyl)carbamate

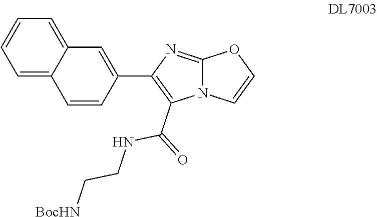 [SMILES: CC(C)(C)OC(=O)NCCNC(=O)c1c(-c2ccc3ccccc3c2)nc2occn12]

¹H NMR (400 MHz, CDCl₃): δ 8.18 (s, 1H), 7.99-7.89 (m, 4H), 7.76 (d, J=7.6 Hz, 1H), 7.57-7.55 (m, 1H), 7.49 (d, J=1.6 Hz, 1H), 6.22 (br s, 1H), 4.79 (br s, 1H), 3.39 (d, J=5.6 Hz, 2H), 3.20 (d, J=5.2 Hz, 2H), 1.35 (s, 9H).

### N-(2-Isothiocyanatoethyl)-6-(naphthalen-2-yl)imidazo[2,1-b]oxazole-5-carboxamide

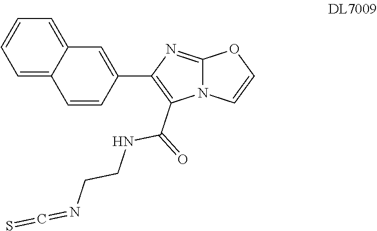 [SMILES: O=C(NCCN=C=S)c1c(-c2ccc3ccccc3c2)nc2occn12]

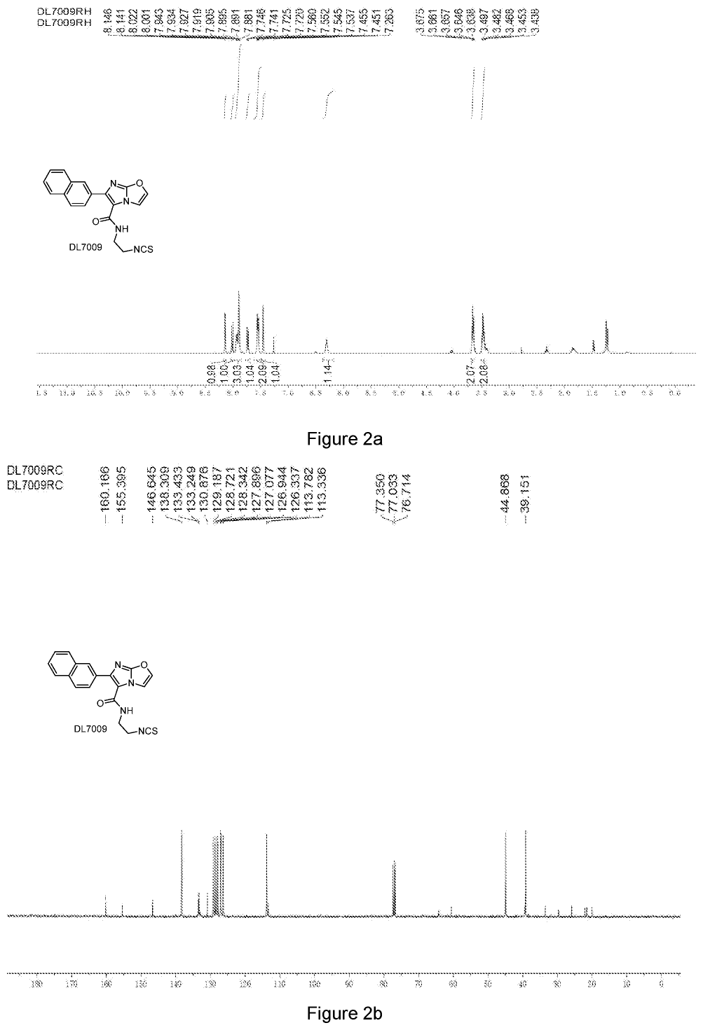

¹H NMR (400 MHz, CDCl₃): δ 8.14 (s, 1H), 8.01 (d, J=8.0 Hz, 1H), 7.94-7.89 (m, 3H), 7.73 (d, J=8.8 Hz, 1H), 7.56-7.54 (m, 2H), 7.45 (s, 1H), 6.31 (d, J=5.6 Hz, 1H), 3.66 (t, J=5.6 Hz, 2H), 3.50-3.45 (m, 2H); ¹³C NMR (100 MHz, CDCl₃): δ 160.2, 155.4, 146.7, 138.3, 133.5, 133.3, 133.0, 130.9, 129.2, 128.7, 128.3, 127.9, 127.1, 127.0, 126.4, 113.8, 113.4, 44.9, 39.2. HRMS (ESI): Exact mass calcd for C₁₉H₁₅N₄O₂S [M+H]⁺ 363.0916, found 363.0908. The ¹H NMR spectrum is shown in FIG. 2a and the ¹³C NMR spectrum is shown in FIG. 2b.

### Tert-butyl (1-(6-(naphthalen-2-yl)imidazo[2,1-b]oxazole-5-carboxamido)propan-2-yl)carbamate

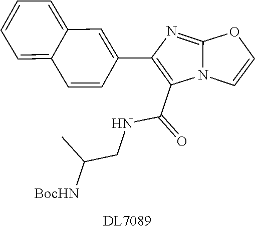 [SMILES: CC(CNC(=O)c1c(-c2ccc3ccccc3c2)nc2occn12)NC(=O)OC(C)(C)C]

¹H NMR (400 MHz, CDCl₃): δ 8.15 (s, 1H), 7.95-7.85 (m, 4H), 7.73 (d, J=8.8 Hz, 1H), 7.54-7.51 (m, 2H), 7.43 (s, 1H), 6.30 (br s, 1H), 4.65 (d, J=6.4 Hz, 1H), 3.69 (br s, 1H), 3.37 (br s, 1H), 3.24 (br s, 1H), 1.31 (s, 9H), 1.05 (d, J=6.4 Hz, 3H).

### N-(2-Isothiocyanatopropyl)-6-(naphthalen-2-yl)imidazo[2,1-b]oxazole-5-carboxamide

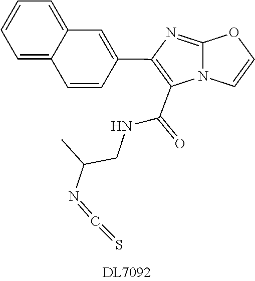 [SMILES: CC(CNC(=O)c1c(-c2ccc3ccccc3c2)nc2occn12)N=C=S]

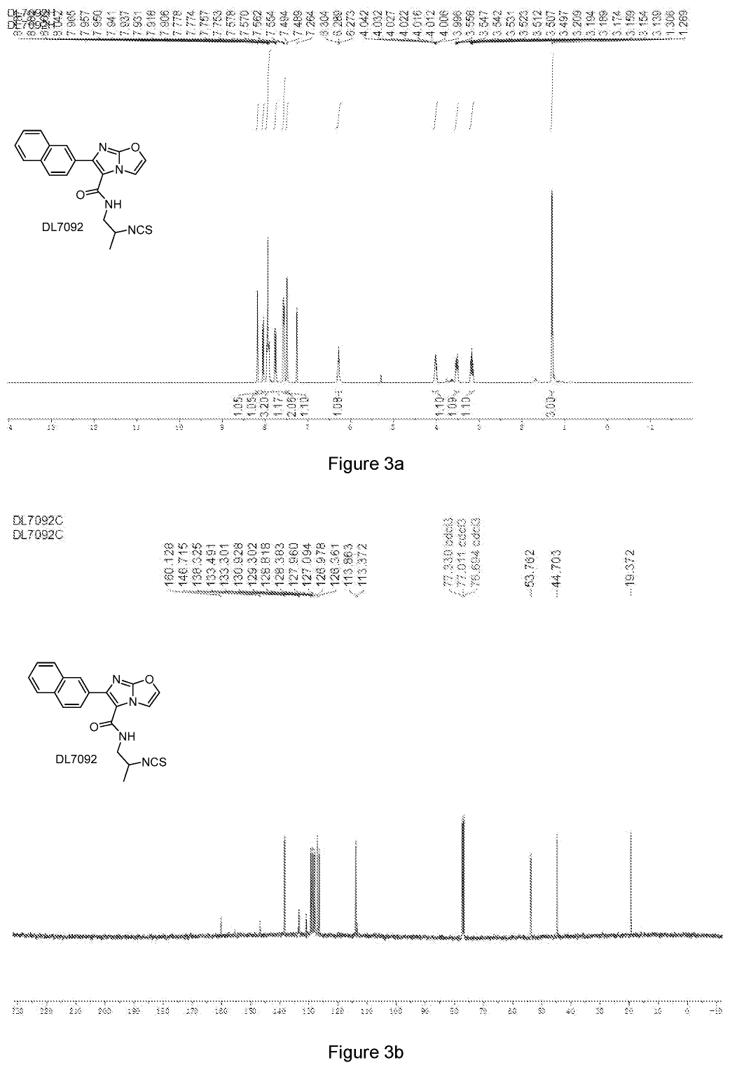

¹H NMR (400 MHz, CDCl₃): δ 8.18 (s, 1H), 8.05 (d, J=8.8 Hz, 1H), 7.97-7.91 (m, 3H), 7.77 (d, J=8.0 Hz, 1H), 7.58-7.55 (m, 2H), 7.49 (d, J=1.6 Hz, 1H), 6.29 (br s, 1H), 4.04-3.98 (m, 1H), 3.56-3.50 (m, 1H), 3.21-3.14 (m, 1H), 1.30 (d, J=6.4 Hz, 3H); ¹³C NMR (100 MHz, CDCl₃): δ 160.1, 157.5, 155.5, 146.7, 138.3, 133.5, 133.3, 130.9, 129.3, 128.8, 128.4, 128.0, 127.1, 127.0, 126.4, 113.9, 113.4, 53.8, 44.7, 19.4. HRMS (ESI): Exact mass calcd for C₂₀H₁₇N₄O₂S [M+H]⁺ 377.1072, found 377.1066. The ¹H NMR spectrum is shown in FIG. 3a and the ¹³C NMR spectrum is shown in FIG. 3b.

### Tert-butyl (2-(6-(naphthalen-2-yl)imidazo[2,1-b]oxazole-5-carboxamido)propyl)carbamate

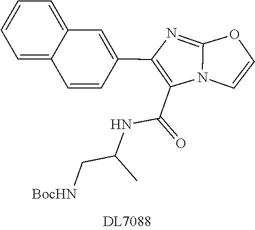 [SMILES: CC(CNC(=O)OC(C)(C)C)NC(=O)c1c(-c2ccc3ccccc3c2)nc2occn12]

¹H NMR (400 MHz, CDCl₃): δ 8.14 (s, 1H), 7.94-7.86 (m, 4H), 7.74 (d, J=9.6 Hz, 1H), 7.54-7.52 (m, 2H), 7.40 (s, 1H), 6.11 (d, J=4.0 Hz, 1H), 4.92 (br s, 1H), 4.20-4.13 (m, 1H), 3.17-3.11 (m, 1H), 3.06-2.99 (m, 1H), 1.33 (s, 9H), 1.00 (d, J=7.2 Hz, 3H).

### N-(1-Isothiocyanatopropan-2-yl)-6-(naphthalen-2-yl)imidazo[2,1-b]oxazole-5-carboxamide

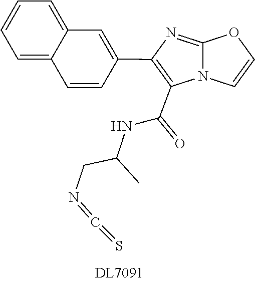 [SMILES: CC(CN=C=S)NC(=O)c1c(-c2ccc3ccccc3c2)nc2occn12]

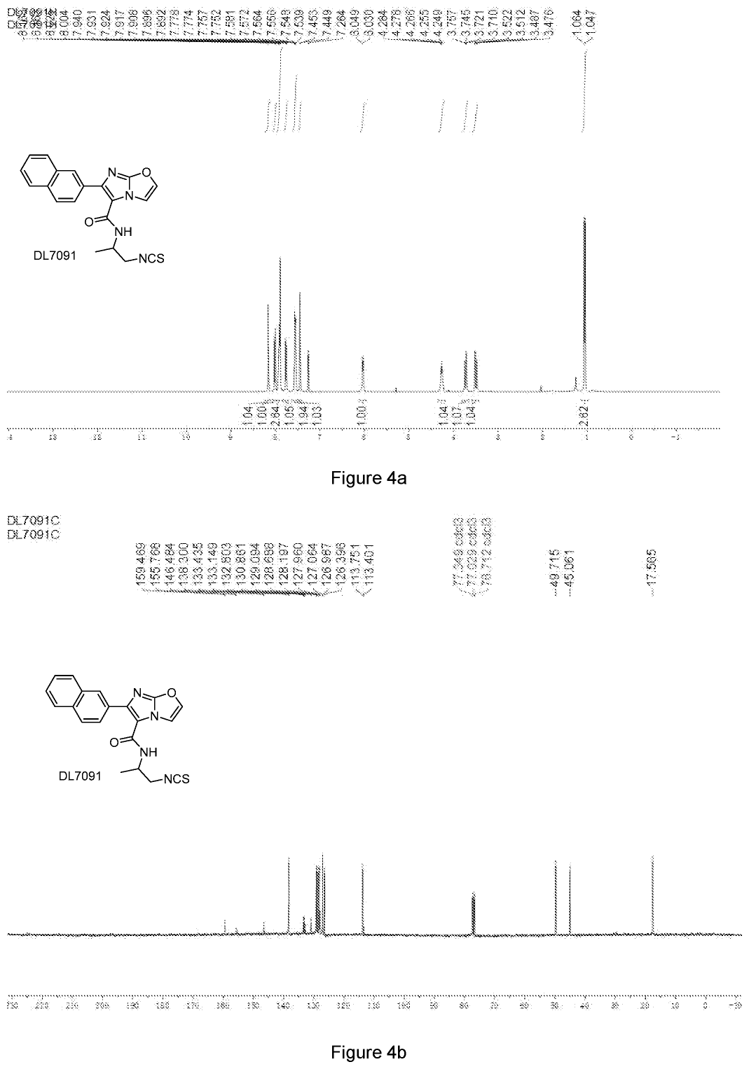

¹H NMR (400 MHz, CDCl₃): δ 8.17 (s, 1H), 8.01 (d, J=8.8 Hz, 1H), 7.94-7.89 (m, 3H), 7.77 (d, J=8.4 Hz, 1H), 7.57-7.55 (m, 2H), 7.45 (s, 1H), 6.04 (d, J=7.2 Hz, 1H), 4.30-4.24 (m, 1H), 3.76-3.71 (m, 1H), 3.52-3.48 (m, 1H), 1.05 (d, J=6.4 Hz, 3H); ¹³C NMR (100 MHz, CDCl₃): δ 159.5, 155.4, 146.5, 138.3, 133.4, 133.2, 132.8, 130.9, 129.1, 128.7, 128.2, 128.0, 127.1, 127.0, 126.4, 113.8, 113.4, 49.7, 45.1, 17.6. HRMS (ESI): Exact mass calcd for C₂₀H₁₇N₄O₂S [M+H]⁺ 377.1072, found 377.1078. The ¹H NMR spectrum is shown in FIG. 4a and the ¹³C NMR spectrum is shown in FIG. 4b.

### Tert-butyl (2-(6-(naphthalen-2-yl)imidazo[2,1-b]oxazole-5-carboxamido)-1-phenylethyl)carbamate

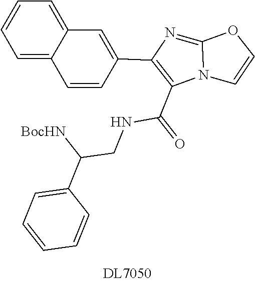 [SMILES: CC(C)(C)OC(=O)NC(CNC(=O)c1c(-c2ccc3ccccc3c2)nc2occn12)c1ccccc1]

¹H NMR (400 MHz, CDCl₃): δ 8.02 (s, 1H), 7.90 (s, 1H), 7.82 (d, J=8.8 Hz, 3H), 7.57-7.51 (m, 3H), 7.42 (s, 1H), 7.16-7.10 (m, 5H), 6.22 (d, J=5.6 Hz, 1H), 5.54 (d, J=4.4 Hz, 1H), 4.74 (br s, 1H), 3.65 (br s, 1H), 3.54-3.48 (m, 1H), 1.36 (s, 9H).

### N-(2-Isothiocyanato-2-phenylethyl)-6-(naphthalen-2-yl)imidazo[2,1-b]oxazole-5-carboxamide

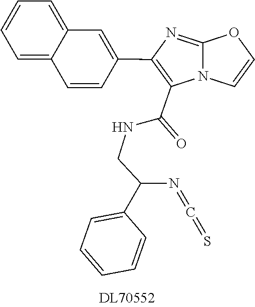 [SMILES: O=C(NCC(N=C=S)c1ccccc1)c1c(-c2ccc3ccccc3c2)nc2occn12]

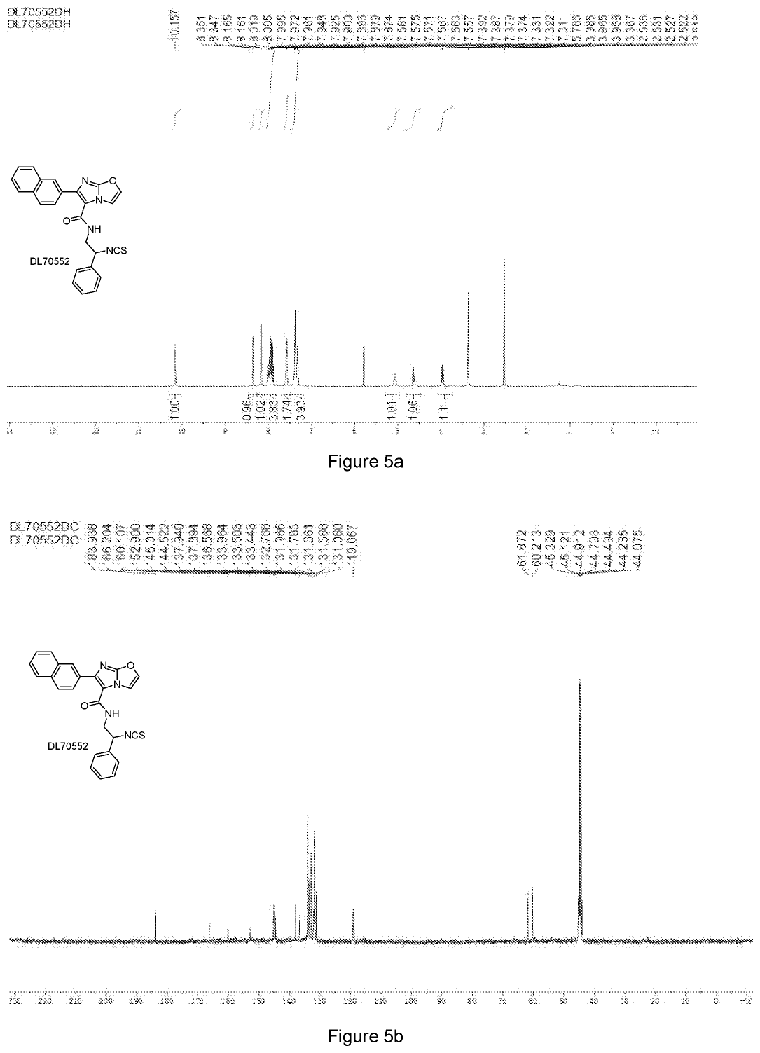

¹H NMR (400 MHz, DMSO-d6): δ 10.16 (s, 1H), 8.35 (s, 1H), 8.16 (d, J=1.6 Hz, 1H), 8.01-7.88 (m, 5H), 7.58-7.56 (m, 2H), 7.38-7.33 (m, 5H), 5.07 (br s, 1H), 4.63 (t, J=10.4 Hz, 1H), 3.99-3.94 (m, 1H); ¹³C NMR (100 MHz, DMSO-d6): δ 184.0, 166.2, 160.1, 152.9, 145.0, 144.5, 137.9, 136.6, 134.0, 133.5, 133.45, 132.8, 132.0, 131.8, 131.7, 131.6, 131.1, 119.1, 61.9, 60.2. HRMS (ESI): Exact mass calcd for C₂₅H₁₉N₄O₂S [M+H]⁺ 439.1229, found 439.1232. The ¹H NMR spectrum is shown in FIG. 5a and the ¹³C NMR spectrum is shown in FIG. 5b.

### Tert-butyl N-(2-isothiocyanato-1-phenylethyl)-6-(naphthalen-2-yl)imidazo[2,1-b]oxazole-5-carboxamide

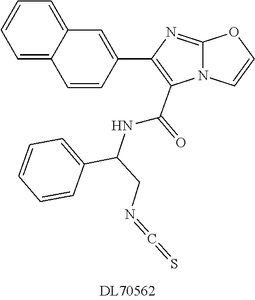 [SMILES: O=C(NC(CN=C=S)c1ccccc1)c1c(-c2ccc3ccccc3c2)nc2occn12]

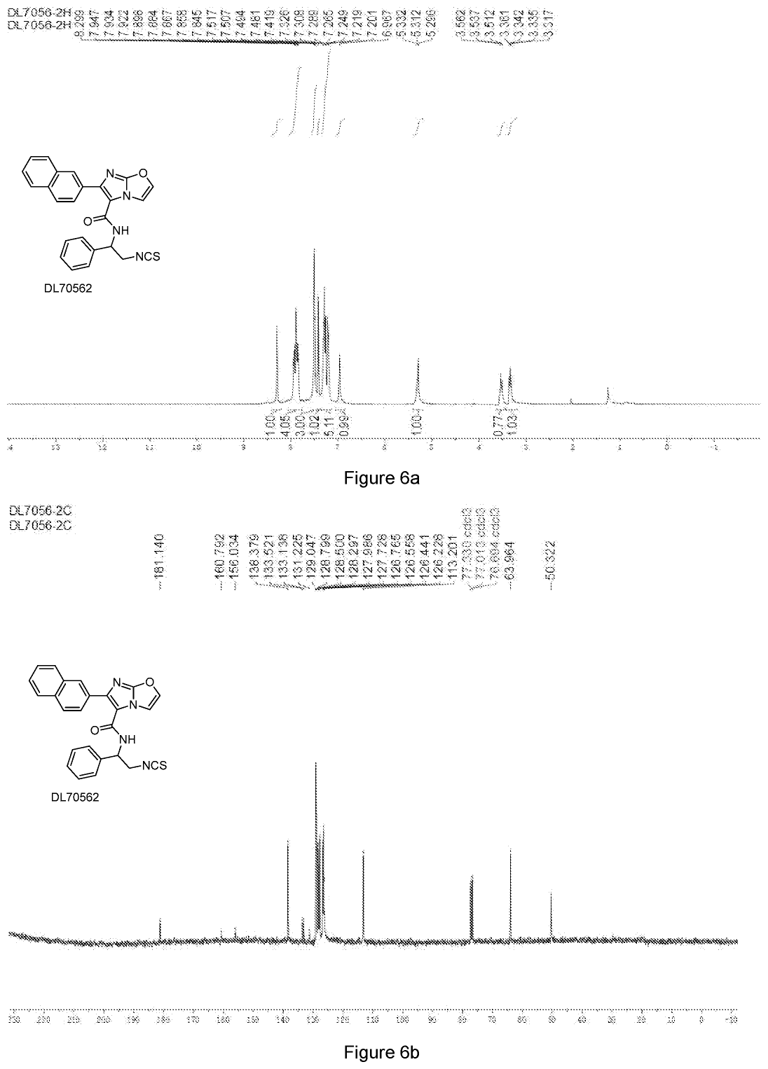

¹H NMR (400 MHz, CDCl₃): δ 8.30 (s, 1H), 7.95-7.85 (m, 4H), 7.51 (s, 3H), 7.42 (s, 1H), 7.31-7.20 (m, 5H), 6.97 (s, 1H), 5.30 (s, 1H), 3.54 (t, J=8.4 Hz, 1H), 3.34 (t, J=10.0 Hz, 1H); ¹³C NMR (100 MHz, CDCl₃): δ 181.2, 160.8, 156.1, 138.4, 133.5, 133.2, 131.3, 129.1, 128.8, 128.5, 128.3, 128.0, 127.7, 126.8, 126.6, 126.5, 126.3, 113.2, 64.0, 50.3. HRMS (ESI): Exact mass calcd for C₂₅H₁₉N₄O₂S [M+H]⁺ 439.1229, found 439.1228. The ¹H NMR spectrum is shown in FIG. 6a and the ¹³C NMR spectrum is shown in FIG. 6b.

### Tert-butyl (1-(6-(naphthalen-2-yl)imidazo[2,1-b]oxazole-5-carbonyl)piperidin-4-yl)carbamate

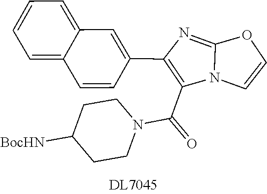 [SMILES: CC(C)(C)OC(=O)NC1CCN(C(=O)c2c(-c3ccc4ccccc4c3)nc3occn23)CC1]

¹H NMR (400 MHz, CDCl₃): δ 8.11 (s, 1H), 7.89-7.83 (m, 3H), 7.73 (d, J=8.0 Hz, 1H), 7.60 (s, 1H), 7.52-7.46 (m, 3H).

### (4-Isothiocyanatopiperidin-1-yl)(6-(naphthalen-2-yl)imidazo[2,1-b]oxazol-5-yl)methanone

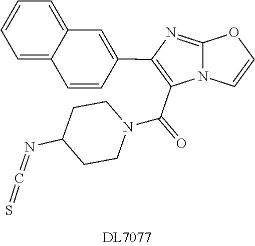 [SMILES: O=C(c1c(-c2ccc3ccccc3c2)nc2occn12)N1CCC(N=C=S)CC1]

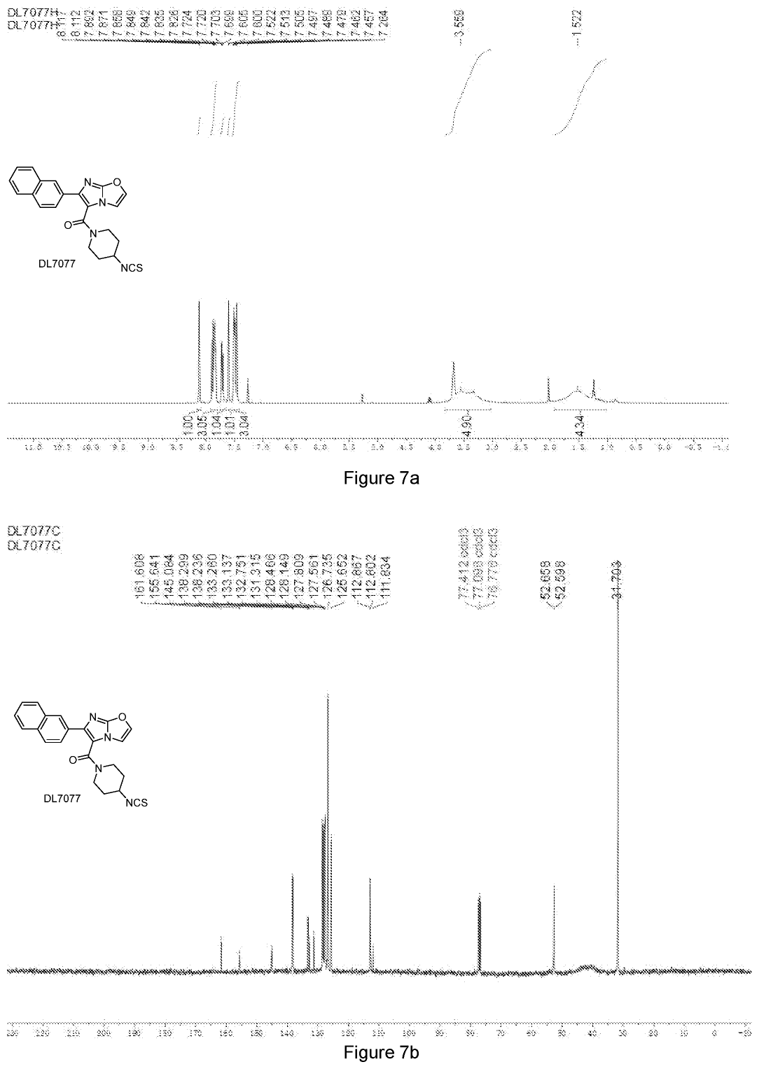

¹H NMR (400 MHz, CDCl₃): δ 8.10 (s, 1H), 7.88-7.82 (m, 3H), 7.70 (d, J=8.4 Hz, 1H), 7.59 (s, 1H), 7.50-7.45 (m, 3H), 3.55 (br s, 4H), 1.51 (br s, 4H); ¹³C NMR (100 MHz, CDCl₃): δ 161.6, 155.6, 145.1, 138.31, 138.26, 133.1, 132.7, 131.3, 128.5, 128.2, 127.8, 127.5, 126.7, 125.7, 112.8, 111.8, 52.6, 31.7. HRMS (ESI): Exact mass calcd for C₂₂H₁₉N₄O₂S [M+H]⁺ 403.1229, found 403.1235. The ¹H NMR spectrum is shown in FIG. 7a and the ¹³C NMR spectrum is shown in FIG. 7b.

### Tert-butyl ((1r,3r)-3-(6-(naphthalen-2-yl)imidazo[2,1-b]oxazole-5-carboxamido)cyclobutyl)carbamate

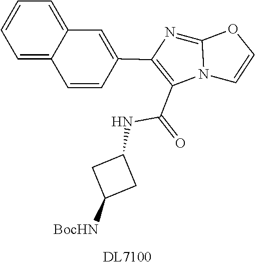 [SMILES: CC(C)(C)OC(=O)N[C@H]1C[C@H](NC(=O)c2c(-c3ccc4ccccc4c3)nc3occn23)C1]

¹H NMR (400 MHz, CDCl₃): δ 8.09 (s, 1H), 7.90 (t, J=7.2 Hz, 2H), 7.84-7.82 (m, 2H), 7.77 (s, 1H), 7.70 (d, J=8.4 Hz, 1H), 7.51-7.48 (m, 2H), 7.33 (d, J=1.6 Hz, 1H), 6.39 (d, J=6.4 Hz, 1H), 4.99 (br s, 1H), 4.46 (d, J=6.4 Hz, 1H), 3.96 (br s, 1H), 2.26-2.20 (m, 2H), 2.06-2.03 (m, 2H), 1.36 (s, 9H).

### N-((1r,3r)-3-Isothiocyanatocyclobutyl)-6-(naphthalen-2-yl)imidazo[2,1-b]oxazole-5-carboxamide

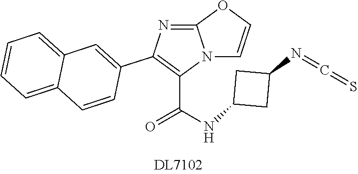 [SMILES: O=C(N[C@H]1C[C@H](N=C=S)C1)c1c(-c2ccc3ccccc3c2)nc2occn12]

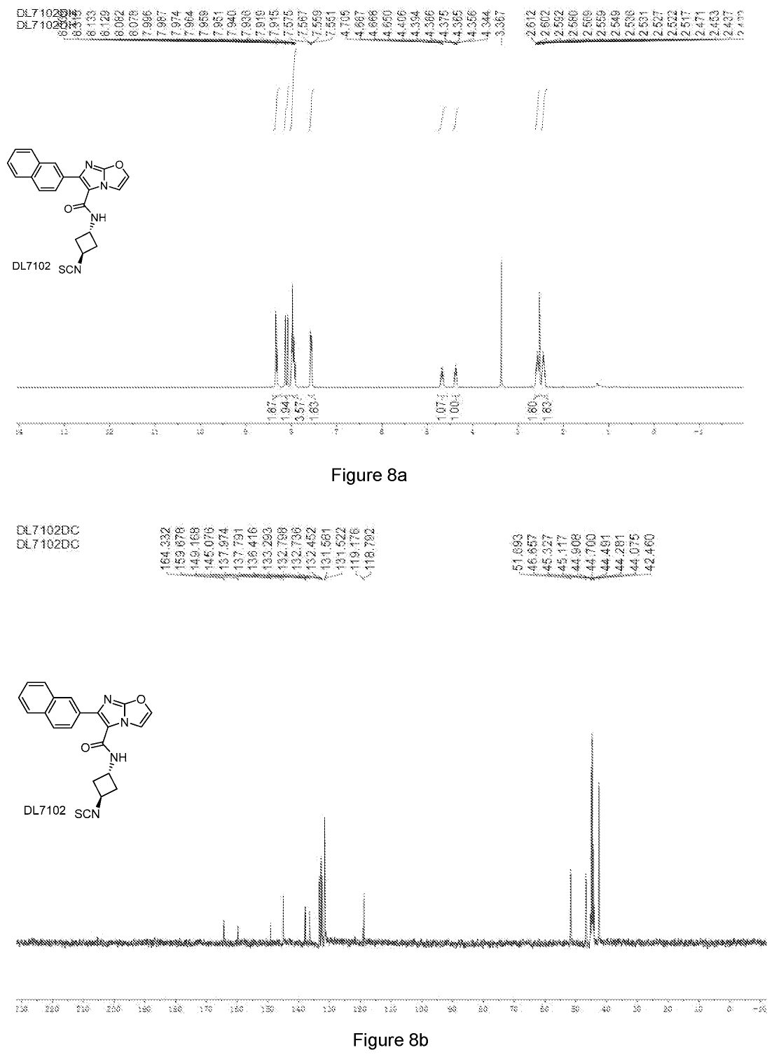

¹H NMR (400 MHz, DMSO-d6): δ 8.32 (d, J=8.8 Hz, 2H), 8.13 (s, 1H), 8.08 (s, 1H), 8.00-7.92 (m, 4H), 7.58-7.55 (m, 2H), 4.70-4.65 (m, 1H), 4.39-4.37 (m, 1H), 2.58-2.55 (m, 2H), 2.47-2.40 (m, 2H); ¹³C NMR (100 MHz, DMSO-d6): δ 164.4, 159.7, 149.2, 145.1, 138.0, 137.8, 136.4, 133.3, 132.8, 132.75, 132.5, 131.6, 119.2, 118.8, 51.7, 46.7, 42.5. HRMS (ESI): Exact mass calcd for C21H17N₄O₂S [M+H]⁺ 389.1072, found 389.1072. The ¹H NMR spectrum is shown in FIG. 8a and the ¹³C NMR spectrum is shown in FIG. 8b.

### Tert-butyl (4-(6-(naphthalen-2-yl)imidazo[2,1-b]oxazole-5-carboxamido)cyclohexyl)carbamate

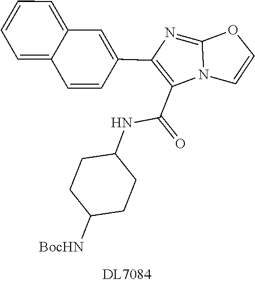 [SMILES: CC(C)(C)OC(=O)NC1CCC(NC(=O)c2c(-c3ccc4ccccc4c3)nc3occn23)CC1]

¹H NMR (400 MHz, CDCl₃): δ 8.14 (s, 1H), 7.99-7.83 (m, 4H), 7.75 (d, J=6.8 Hz, 1H), 7.59-7.55 (m, 2H), 5.89 (d, J=7.2 Hz, 1H), 4.40 (s, 1H), 3.85-3.83 (m, 1H), 3.25 (br s, 1H), 1.93 (br s, 4H), 1.41 (s, 9H), 1.25-1.15 (m, 2H), 1.03-0.94 (m, 2H).

### N-(4-Isothiocyanatocyclohexyl)-6-(naphthalen-2-yl)imidazo[2,1-b]oxazole-5-carboxamide

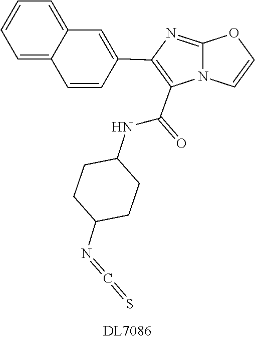 [SMILES: O=C(NC1CCC(N=C=S)CC1)c1c(-c2ccc3ccccc3c2)nc2occn12]

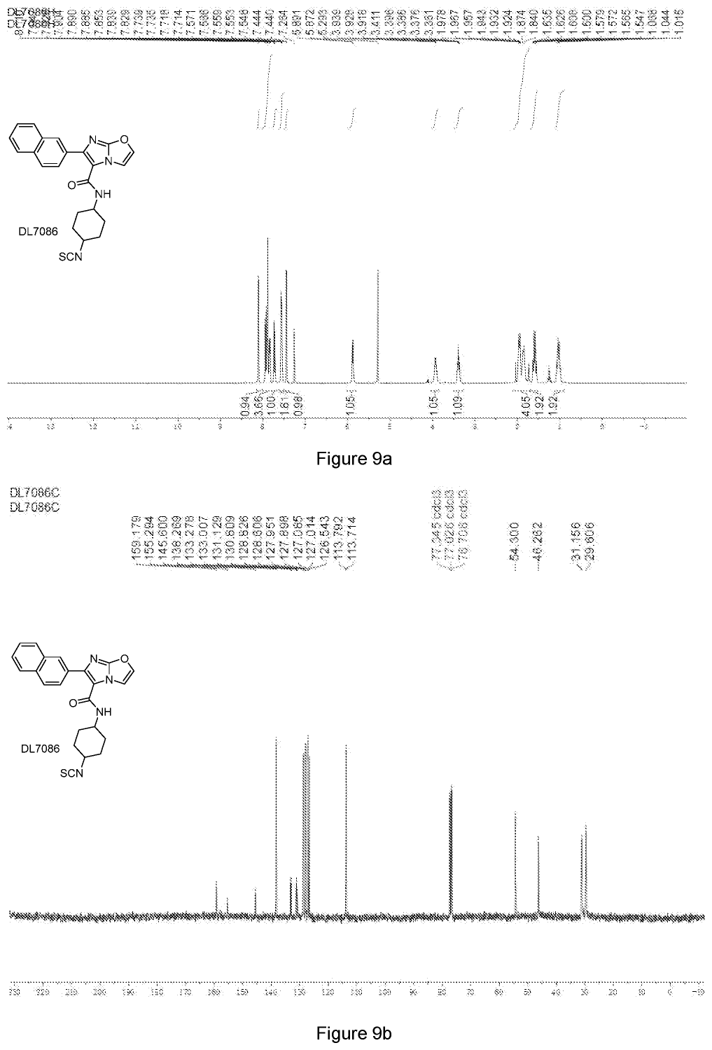

¹H NMR (400 MHz, CDCl₃): δ 8.11 (s, 1H), 7.95-7.83 (m, 4H), 7.73 (d, J=8.0 Hz, 1H), 7.57-7.55 (m, 2H), 7.44 (d, J=1.6 Hz, 1H), 5.88 (d, J=8.0 Hz, 1H), 3.94-3.91 (m, 1H), 3.41-3.36 (m, 1H), 1.97-1.84 (m, 4H), 1.63-1.54 (m, 2H), 1.07-0.99 (m, 2H); ¹³C NMR (100 MHz, CDCl₃): δ 159.2, 155.3, 145.6, 138.3, 133.3, 133.0, 131.1, 130.8, 128.8, 128.6, 128.0, 127.9, 127.1, 127.0, 126.6, 113.7, 54.3, 46.3, 31.2, 29.6. HRMS (ESI): Exact mass calcd for C₂₃H₂₁N₄O₂S [M+H]⁺ 417.1385, found 417.1384. The ¹H NMR spectrum is shown in FIG. 9a and the ¹³C NMR spectrum is shown in FIG. 9b.

### Tert-butyl ((1R,2R)-2-(6-(Naphthalen-2-yl)imidazo[2,1-b]oxazole-5-carboxamido)cyclohexyl)carbamate

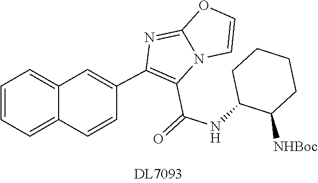 [SMILES: CC(C)(C)OC(=O)N[C@@H]1CCCC[C@H]1NC(=O)c1c(-c2ccc3ccccc3c2)nc2occn12]

¹H NMR (400 MHz, CDCl₃): δ 8.21 (s, 1H), 7.98-7.88 (m, 4H), 7.80-7.77 (m, 1H), 7.55-7.52 (m, 2H), 7.45 (d, J=1.6 Hz, 1H), 6.23 (d, J=8.8 Hz, 1H), 4.92 (d, J=8.8 Hz, 1H), 3.86-3.77 (m, 1H), 3.14 (br s, 1H), 2.04-1.87 (m, 3H), 1.66-1.63 (m, 2H), 1.29 (s, 9H), 1.18-1.13 (m, 2H), 0.99-0.90 (m, 1H); ¹³C NMR (100 MHz, CDCl₃): δ 160.2, 156.0, 155.3, 146.1, 138.1, 133.4, 133.2, 130.9, 128.9, 128.6, 128.3, 127.8, 126.8, 126.7, 126.6, 113.7, 79.1, 55.0, 52.8, 32.8, 32.2, 28.2, 24.7, 24.5.

### N-((1R,2R)-2-Isothiocyanatocyclohexyl)-6-(naphthalen-2-yl)imidazo[2,1-b]oxazole-5-carboxamide

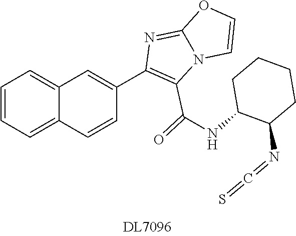 [SMILES: O=C(N[C@@H]1CCCC[C@H]1N=C=S)c1c(-c2ccc3ccccc3c2)nc2occn12]

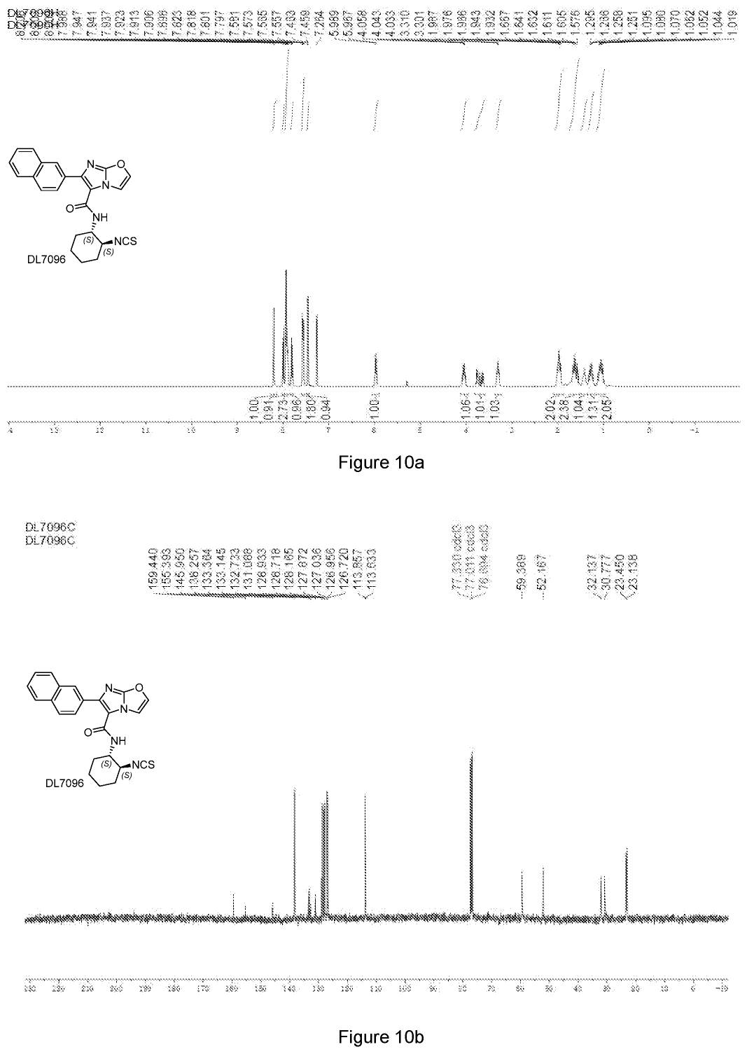

¹H NMR (400 MHz, CDCl₃): δ 8.21 (s, 1H), 8.00 (d, J=8.0 Hz, 1H), 7.94-7.90 (m, 3H), 7.81 (d, J=8.4 Hz, 1H), 7.58-7.57 (m, 2H), 7.46 (s, 1H), 5.98 (d, J=9.6 Hz, 1H), 4.09-4.01 (m, 1H), 3.78-3.62 (m, 1H), 3.31 (t, J=6.4 Hz, 1H), 2.01-1.93 (m, 2H), 1.67-1.54 (m, 2H), 1.41 (br s, 1H), 1.32-1.23 (m, 1H), 1.12-0.98 (m, 2H); ¹³C NMR (100 MHz, CDCl₃): δ 159.5, 155.8, 146.0, 138.3, 133.4, 133.2, 132.6, 131.1, 128.9, 128.7, 128.2, 127.9, 127.1, 127.0, 126.7, 113.9, 113.6, 59.4, 52.2, 32.1, 30.8, 23.5, 23.1. HRMS (ESI): Exact mass calcd for C₂₃H₂₁N₄O₂S [M+H]⁺ 417.1385, found 417.1394. The ¹H NMR spectrum is shown in FIG. 10a and the ¹³C NMR spectrum is shown in FIG. 10b.

### Tert-butyl ((1S,2S)-2-(6-(naphthalen-2-yl)imidazo[2,1-b]oxazole-5-carboxamido)cyclopentyl)carbamate

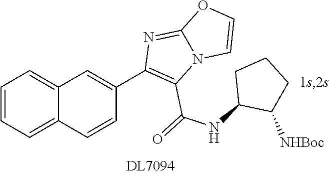 [SMILES: CC(C)(C)OC(=O)N[C@H]1CCC[C@@H]1NC(=O)c1c(-c2ccc3ccccc3c2)nc2occn12]

¹H NMR (400 MHz, CDCl₃): δ 8.11 (s, 1H), 7.96-7.81 (m, 3H), 7.76 (d, J=1.6 Hz, 1H), 7.72-7.69 (m, 1H), 7.52-7.48 (m, 2H), 7.29 (d, J=1.6 Hz, 1H), 6.46 (d, J=7.6 Hz, 1H), 5.11 (d, J=6.8 Hz, 1H), 4.17-4.09 (m, 1H), 3.52 (br s, 1H), 2.08-1.97 (m, 2H), 1.68-1.46 (m, 2H), 1.35 (s, 9H), 1.25-1.15 (m, 1H); ¹³C NMR (100 MHz, CDCl₃): δ 160.3, 155.9, 155.1, 145.8, 138.0, 133.2, 133.1, 130.9, 128.6, 128.4, 128.2, 127.8, 126.7, 126.6, 126.5, 113.6, 79.3, 57.6, 55.7, 30.1, 29.3, 28.3, 19.8.

### N-((1S,2S)-2-Isothiocyanatocyclopentyl)-6-(naphthalen-2-yl)imidazo[2,1-b]oxazole-5-carboxamide

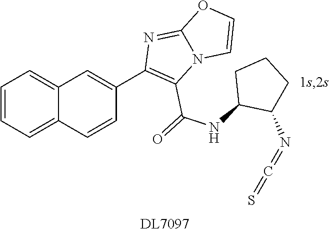 [SMILES: O=C(N[C@H]1CCC[C@@H]1N=C=S)c1c(-c2ccc3ccccc3c2)nc2occn12]

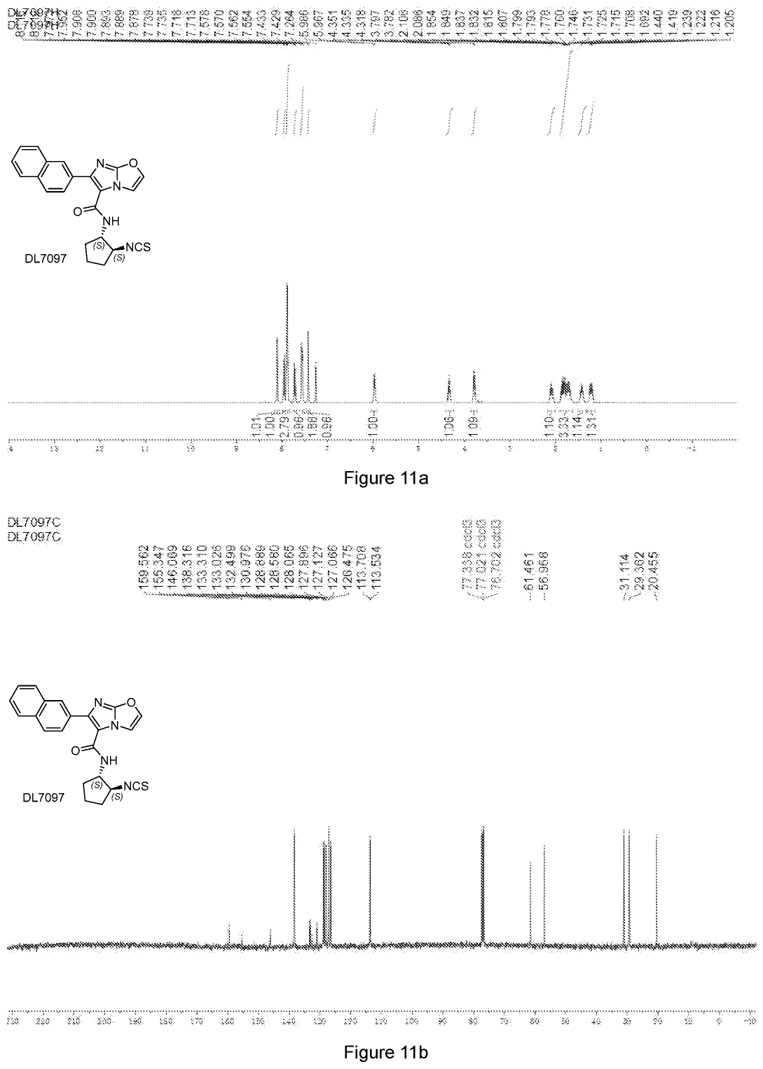

¹H NMR (400 MHz, CDCl₃): δ 8.11 (s, 1H), 7.96 (d, J=8.8 Hz, 1H), 7.89 (s, 3H), 7.73 (d, J=8.8 Hz, 1H), 7.58-7.55 (m, 2H), 7.43 (s, 1H), 5.98 (d, J=8.0 Hz, 1H), 4.37-4.30 (m, 1H), 3.81-3.77 (m, 1H), 2.14-2.05 (m, 1H), 1.91-1.68 (m, 3H), 1.46-1.39 (m, 1H), 1.26-1.17 (m, 1H); ¹³C NMR (100 MHz, CDCl₃): δ 159.6, 155.4, 146.1, 138.3, 133.3, 133.0, 132.5, 131.0, 128.9, 128.6, 128.1, 127.9, 127.14, 127.08, 126.5, 113.7, 113.6, 61.5, 57.0, 31.1, 29.4, 20.5. HRMS (ESI): Exact mass calcd for C₂₂H₁₉N₄O₂S [M+H]⁺ 403.1229, found 403.1226. The ¹H NMR spectrum is shown in FIG. 11 a and the ¹³C NMR spectrum is shown in FIG. 11b.

### Tert-butyl (3-(6-(naphthalen-2-yl)imidazo[2,1-b]oxazole-5-carboxamido)propyl)carbamate

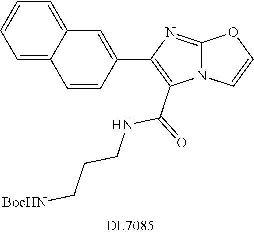 [SMILES: CC(C)(C)OC(=O)NCCCNC(=O)c1c(-c2ccc3ccccc3c2)nc2occn12]

¹H NMR (400 MHz, CDCl₃): δ 8.13 (s, 1H), 7.96-7.85 (m, 4H), 7.73 (d, J=8.8 Hz, 1H), 7.53-7.51 (m, 2H), 7.44 (s, 1H); 6.30 (s, 1H), 4.90 (s, 1H), 3.30 (q, J=6.4 Hz, 2H), 3.05 (d, J=5.6 Hz, 2H), 1.55 (t, J=6.4 Hz, 2H), 1.30 (s, 9H).

### N-(3-Isothiocyanatopropyl)-6-(naphthalen-2-yl)imidazo[2,1-b]oxazole-5-carboxamide

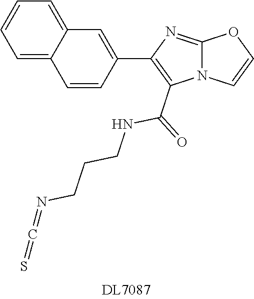 [SMILES: O=C(NCCCN=C=S)c1c(-c2ccc3ccccc3c2)nc2occn12]

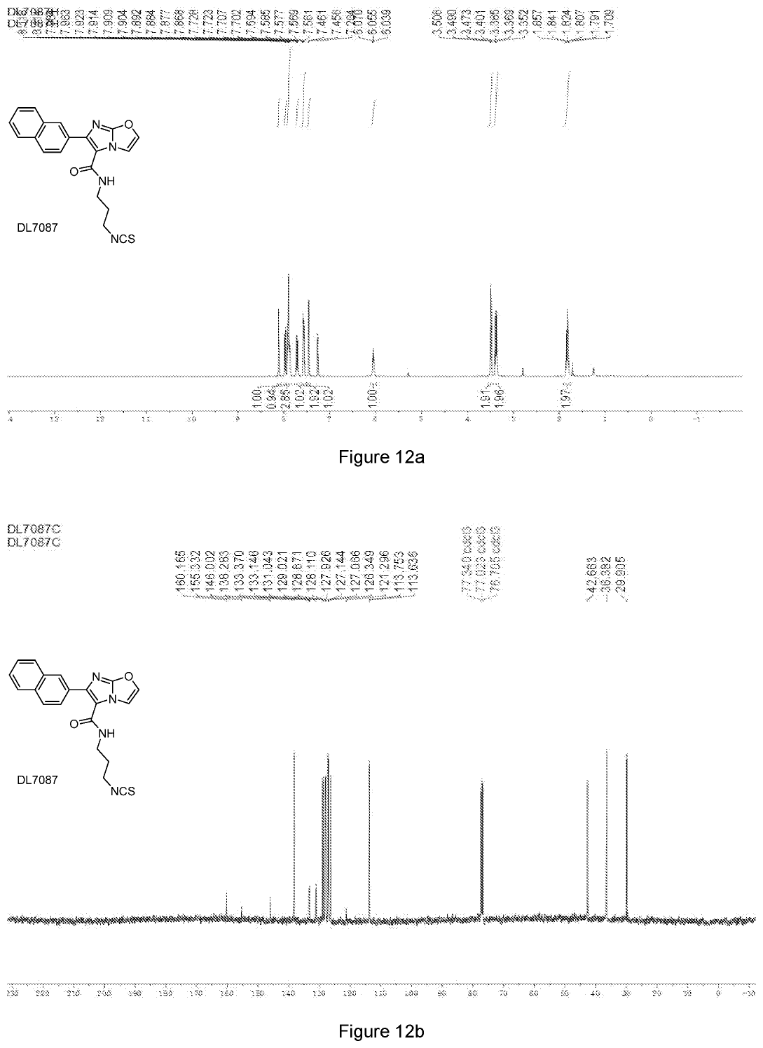

¹H NMR (400 MHz, CDCl₃): δ 8.12 (s, 1H), 7.97 (d, J=8.8 Hz, 1H), 7.92-7.87 (m, 3H), 7.71 (d, J=8.4 Hz, 1H), 7.59-7.56 (m, 2H), 7.46 (s, 1H), 6.05 (br s, 1H), 3.49 (t, J=6.4 Hz, 2H), 3.40-3.35 (m, 2H), 1.86-1.79 (m, 2H); ¹³C NMR (100 MHz, CDCl₃): δ 160.2, 155.3, 146.0, 138.3, 133.4, 133.2, 131.1, 129.0, 128.7, 128.1, 127.9, 127.2, 127.1, 126.4, 121.3, 113.8, 113.6, 42.7, 36.4, 29.9. HRMS (ESI): Exact mass calcd for C₂₀H₁₇N₄O₂S [M+H]⁺ 377.1072, found 377.1076. The ¹H NMR spectrum is shown in FIG. 12a and the ¹³C NMR spectrum is shown in FIG. 12b.

### Tert-butyl (4-(6-(naphthalen-2-yl)imidazo[2,1-b]oxazole-5-carboxamido)butyl)carbamate

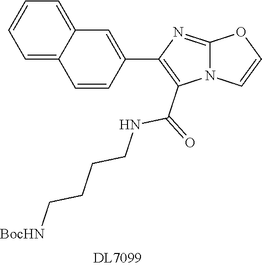 [SMILES: CC(C)(C)OC(=O)NCCCCNC(=O)c1c(-c2ccc3ccccc3c2)nc2occn12]

¹H NMR (400 MHz, CDCl₃): δ 8.10 (s, 1H), 7.96-7.84 (m, 4H), 7.71 (d, J=8.4 Hz, 1H), 7.54-7.52 (m, 2H), 7.43 (d, J=1.6 Hz, 1H), 5.98 (d, J=5.6 Hz, 1H), 4.47 (br s, 1H), 3.24 (q, J=6.0 Hz, 2H), 2.98-2.96 (m, 2H), 1.39-1.31 (m, 11H), 1.29-1.24 (m, 2H).

### N-(4-Isothiocyanatobutyl)-6-(naphthalen-2-yl)imidazo[2,1-b]oxazole-5-carboxamide

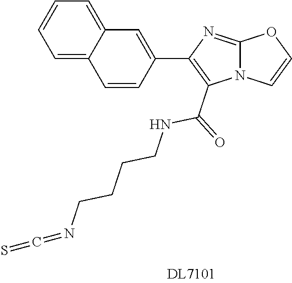 [SMILES: O=C(NCCCCN=C=S)c1c(-c2ccc3ccccc3c2)nc2occn12]

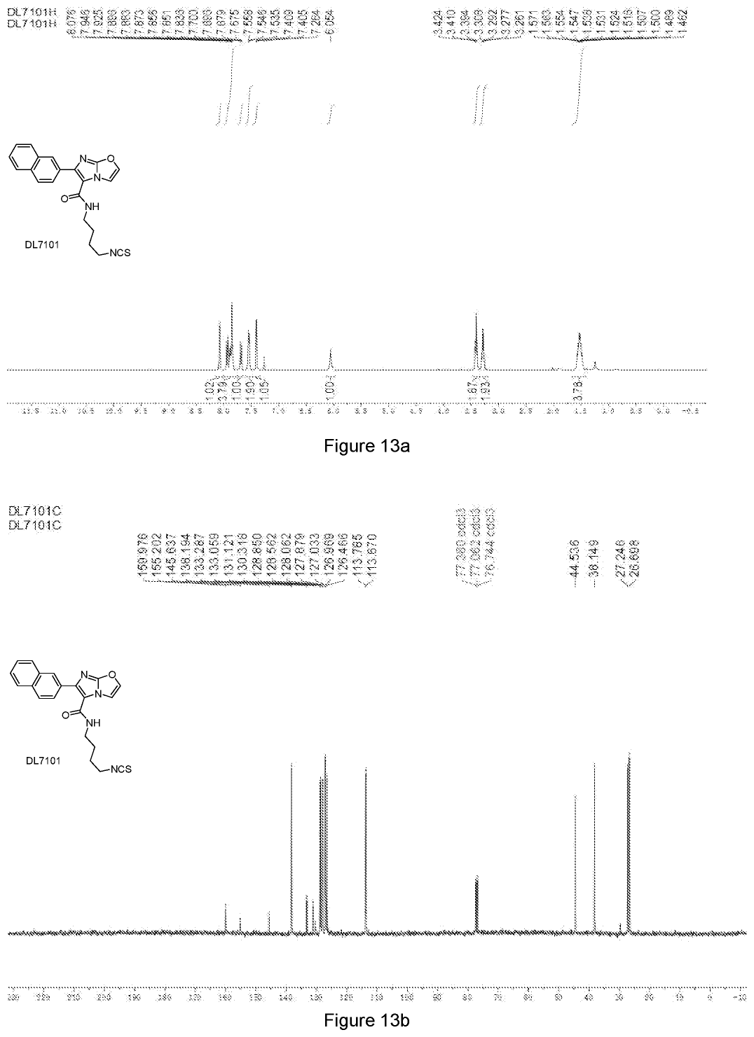

¹H NMR (400 MHz, CDCl₃): δ 8.08 (s, 1H), 7.95-7.84 (m, 4H), 7.69 (d, J=8.8 Hz, 1H), 7.56-7.52 (m, 2H), 7.41 (d, J=1.6 Hz, 1H), 6.05 (br s, 1H), 3.41 (t, J=5.6 Hz, 2H), 3.28 (q, J=6.0 Hz, 2H), 1.57-1.48 (m, 4H); ¹³C NMR (100 MHz, CDCl₃): δ 160.0, 155.2, 145.7, 138.2, 133.3, 133.0, 131.1, 130.3, 128.9, 128.6, 128.1, 127.9, 127.04, 126.98, 126.5, 113.8, 113.7, 44.5, 38.1, 27.3, 26.7. HRMS (ESI): Exact mass calcd for C₂₁H₁₉N₄O₂S [M+H]⁺ 391.1229, found 391.1226. The ¹H NMR spectrum is shown in FIG. 13a and the ¹³C NMR spectrum is shown in FIG. 13b.

### Tert-butyl (2-(6-(naphthalen-2-yl)imidazo[2,1-b]thiazole-5-carboxamido)ethyl)carbamate

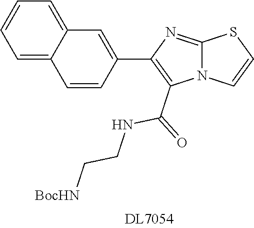 [SMILES: CC(C)(C)OC(=O)NCCNC(=O)c1c(-c2ccc3ccccc3c2)nc2sccn12]

¹H NMR (400 MHz, CDCl₃): δ 8.35 (d, J=3.6 Hz, 1H), 8.18 (s, 1H), 7.99-7.89 (m, 3H), 7.75 (d, J=8.4 Hz, 1H), 7.56 (d, J=4.0 Hz, 2H), 6.95 (d, J=4.0 Hz, 1H), 6.18 (br s, 1H), 4.77 (br s, 1H), 3.40 (t, J=4.8 Hz, 2H), 3.19 (t, J=4.8 Hz, 2H), 1.34 (s, 9H).

### N-(2-Isothiocyanatoethyl)-6-(naphthalen-2-yl)imidazo[2,1-b]thiazole-5-carboxamide

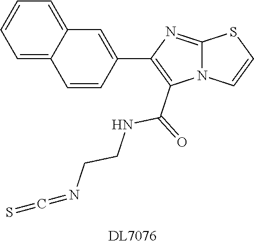 [SMILES: O=C(NCCN=C=S)c1c(-c2ccc3ccccc3c2)nc2sccn12]

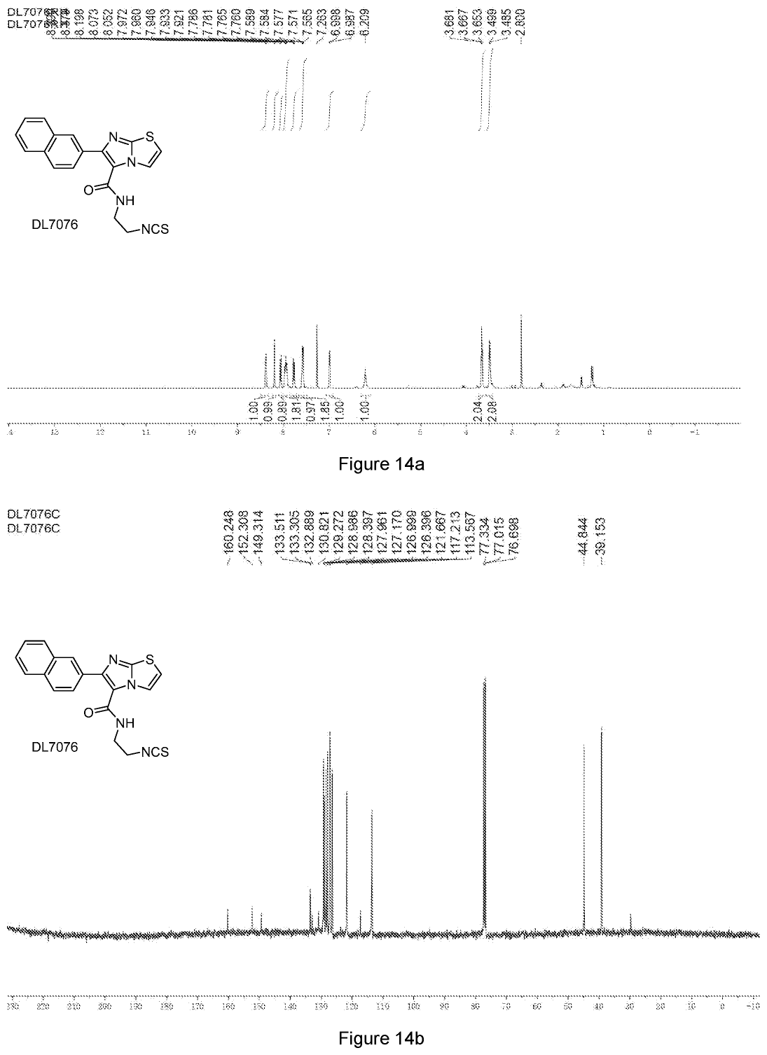

¹H NMR (400 MHz, CDCl₃): δ 8.37 (d, J=4.0 Hz, 1H), 8.19 (s, 1H), 8.05 (d, J=8.8 Hz, 1H), 7.97-7.91 (m, 2H), 7.76 (d, J=8.4 Hz, 1H), 7.59-7.56 (m, 2H), 6.98 (d, J=3.2 Hz, 1H), 6.23 (br s, 1H), 3.66 (t, J=5.6 Hz, 2H), 3.51-3.47 (m, 2H); ¹³C NMR (100 MHz, CDCl₃): δ 160.3, 152.3, 149.3, 133.5, 133.3, 130.8, 129.3, 129.0, 128.4, 128.0, 127.2, 127.0, 126.4, 121.7, 117.2, 113.6, 44.8, 39.2. HRMS (ESI): Exact mass calcd for C₁₉H₁₅N₄OS₂ [M+H]⁺ 379.0687, found 379.0687. The ¹H NMR spectrum is shown in FIG. 14a and the ¹³C NMR spectrum is shown in FIG. 14b.

### Tert-butyl (2-(6-phenylimidazo[2,1-b]thiazole-5-carboxamido)ethyl)carbamate

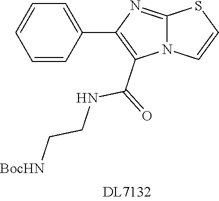 [SMILES: CC(C)(C)OC(=O)NCCNC(=O)c1c(-c2ccccc2)nc2sccn12]

¹H NMR (400 MHz, CDCl₃): δ 8.35 (d, J=4.0 Hz, 1H), 7.66 (d, J=6.8 Hz, 2H), 7.54-7.47 (m, 3H), 6.96 (d, J=4.8 Hz, 1H), 6.05 (s, 1H), 4.79 (s, 1H), 3.43-3.39 (m, 2H), 3.22-3.12 (m, 3H), 1.41 (s, 9H); ¹³C NMR (100 MHz, CDCl₃): δ 160.6, 156.0, 151.9, 149.0, 134.0, 133.9, 129.4, 129.3, 129.1, 121.6, 113.2, 79.2, 40.6, 39.1, 28.3.

### N-(2-Isothiocyanatoethyl)-6-phenylimidazo[2,1-b]thiazole-5-carboxamide

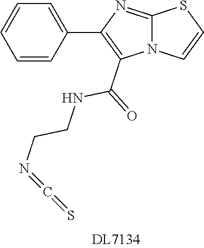 [SMILES: O=C(NCCN=C=S)c1c(-c2ccccc2)nc2sccn12]

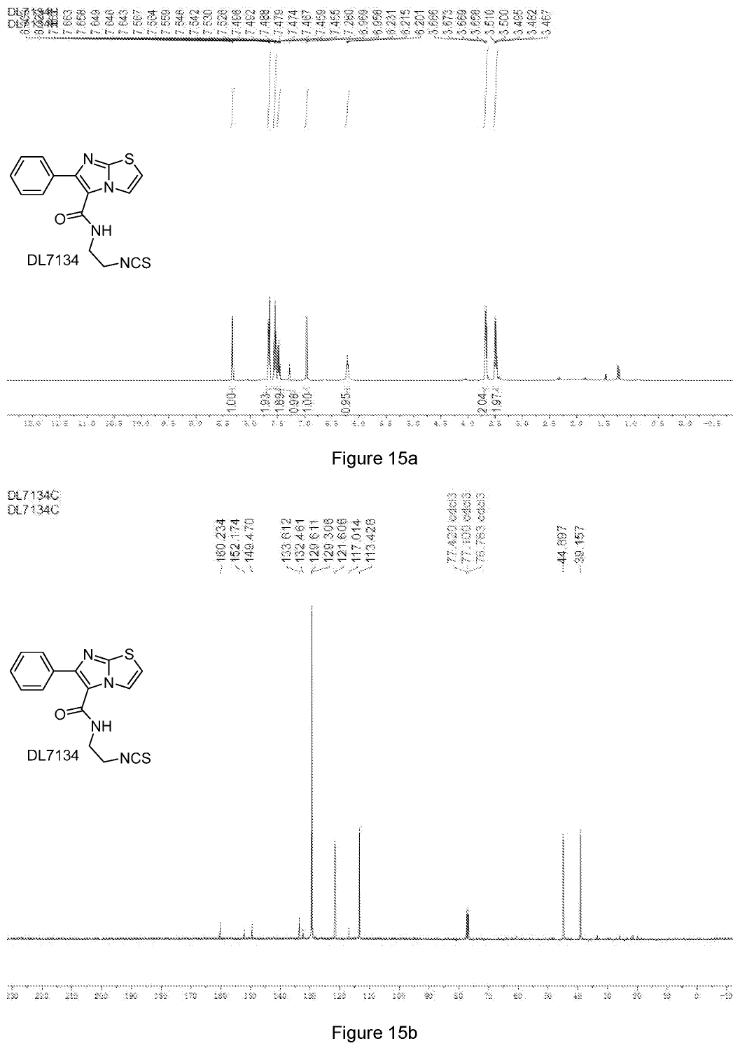

¹H NMR (400 MHz, CDCl₃): δ 8.32 (d, J=4.8 Hz, 1H), 7.65 (d, J=7.2 Hz, 2H), 7.55 (t, J=7.2 Hz, 2H), 7.47 (t, J=6.8 Hz, 1H), 6.96 (d, J=4.8 Hz, 1H), 6.21 (t, J=4.8 Hz, 1H), 3.67 (t, J=4.8 Hz, 2H), 3.49 (q, J=4.8 Hz, 2H); ¹³C NMR (100 MHz, CDCl₃): δ 160.3, 152.2, 149.5, 133.6, 132.5, 129.6, 129.3, 121.6, 117.0, 113.4, 44.9, 39.2. HRMS (ESI): Exact mass calcd for C₁₅H₁₃N₄OS₂ [M+H]⁺ 329.0531, found 329.0537. The ¹H NMR spectrum is shown in FIG. 15a and the ¹³C NMR spectrum is shown in FIG. 15b.

### Tert-butyl (2-(6-(4-fluorophenyl)imidazo[2,1-b]thiazole-5-carboxamido)ethyl)carbamate

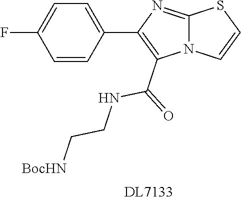 [SMILES: CC(C)(C)OC(=O)NCCNC(=O)c1c(-c2ccc(F)cc2)nc2sccn12]

¹H NMR (400 MHz, CDCl₃): δ 8.28 (d, J=4.4 Hz, 1H), 7.64-7.61 (m, 2H), 7.17 (t, J=8.4 Hz, 2H), 6.93 (d, J=4.8 Hz, 1H), 6.08 (s, 1H), 4.83 (s, 1H), 3.43-3.37 (m, 2H), 3.21-3.17 (m, 2H), 1.37 (s, 9H); ¹³C NMR (100 MHz, CDCl₃): δ 164.5, 162.0, 160.4, 156.1, 151.9, 147.8, 131.3, 131.2, 129.9, 121.5, 117.4, 116.3, 116.1, 113.3, 79.6, 40.6, 39.3, 28.3.

### 6-(4-Fluorophenyl)-N-(2-isothiocyanatoethyl)imidazo[2,1-b]thiazole-5-carboxamide

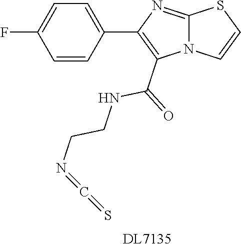 [SMILES: O=C(NCCN=C=S)c1c(-c2ccc(F)cc2)nc2sccn12]

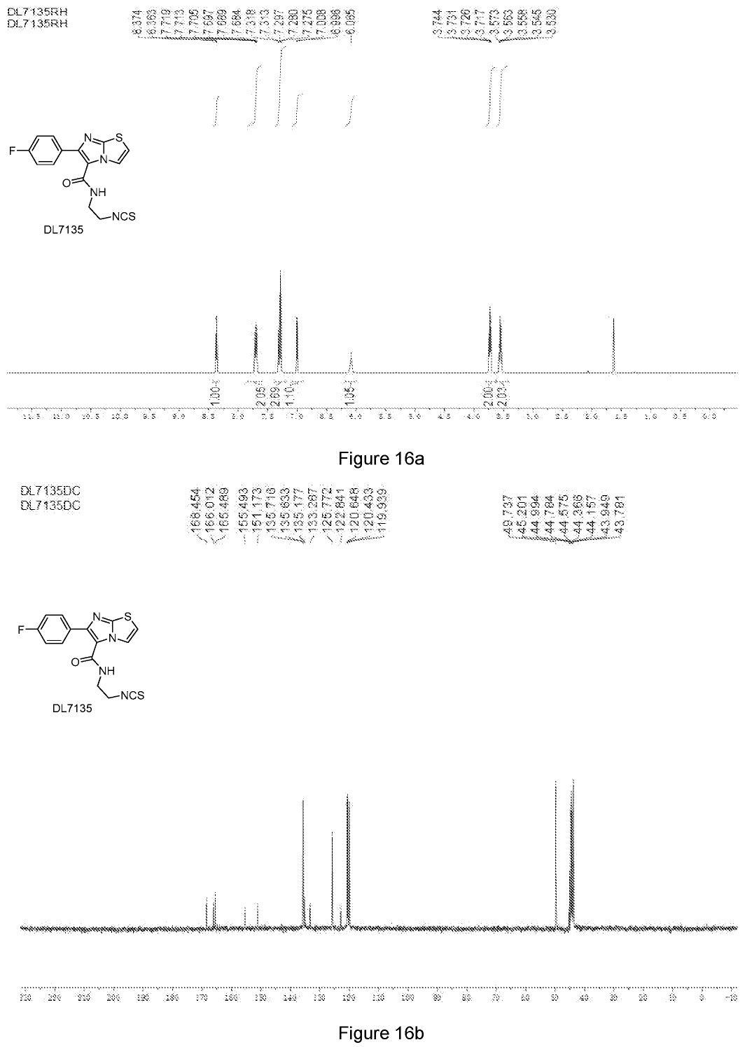

¹H NMR (400 MHz, CDCl₃): δ 8.36 (d, J=4.8 Hz, 1H), 7.71-7.68 (m, 2H), 7.29 (t, J=8.0 Hz, 2H), 7.00 (d, J=4.8 Hz, 1H), 6.09 (s, 1H), 3.73 (t, J=5.6 Hz, 2H), 3.55 (q, J=5.6 Hz, 2H); ¹³C NMR (100 MHz, DMSO-d6): δ 168.5, 166.0, 165.5, 155.5, 151.2, 135.7, 135.6, 135.2, 133.3, 125.8, 122.9, 120.7, 120.4, 120.0, 49.7, 43.8. HRMS (ESI): Exact mass calcd for C₁₅H₁₂FN₄OS₂ [M+H]⁺ 347.0437, found 347.0441. The ¹H NMR spectrum is shown in FIG. 16a and the ¹³C NMR spectrum is shown in FIG. 16b.

### Tert-butyl (2-(6-(4-chlorophenyl)imidazo[2,1-b]thiazole-5-carboxamido)ethyl)carbamate

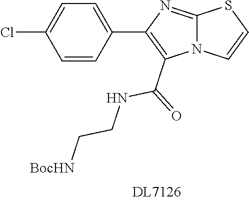 [SMILES: CC(C)(C)OC(=O)NCCNC(=O)c1c(-c2ccc(Cl)cc2)nc2sccn12]

¹H NMR (400 MHz, CDCl₃): δ 8.25 (d, J=4.8 Hz, 1H), 7.58 (d, J=8.4 Hz, 2H), 7.43 (d, J=8.4 Hz, 2H), 6.94 (d, J=4.8 Hz, 1H), 6.25 (s, 1H), 4.95 (s, 1H), 3.45-3.40 (s, 2H), 3.24-3.21 (m, 2H), 1.39 (s, 9H), ¹³C NMR (100 MHz, CDCl₃): δ 160.4, 156.2, 151.9, 147.4, 135.3, 132.1, 130.5, 129.2, 121.5, 117.5, 113.5, 76.6, 40.5, 39.4, 20.3.

### 6-(4-Chlorophenyl)-N-(2-isothiocyanatoethyl)imidazo[2,1-b]thiazole-5-carboxamide

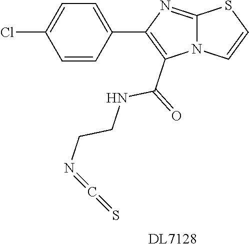 [SMILES: O=C(NCCN=C=S)c1c(-c2ccc(Cl)cc2)nc2sccn12]

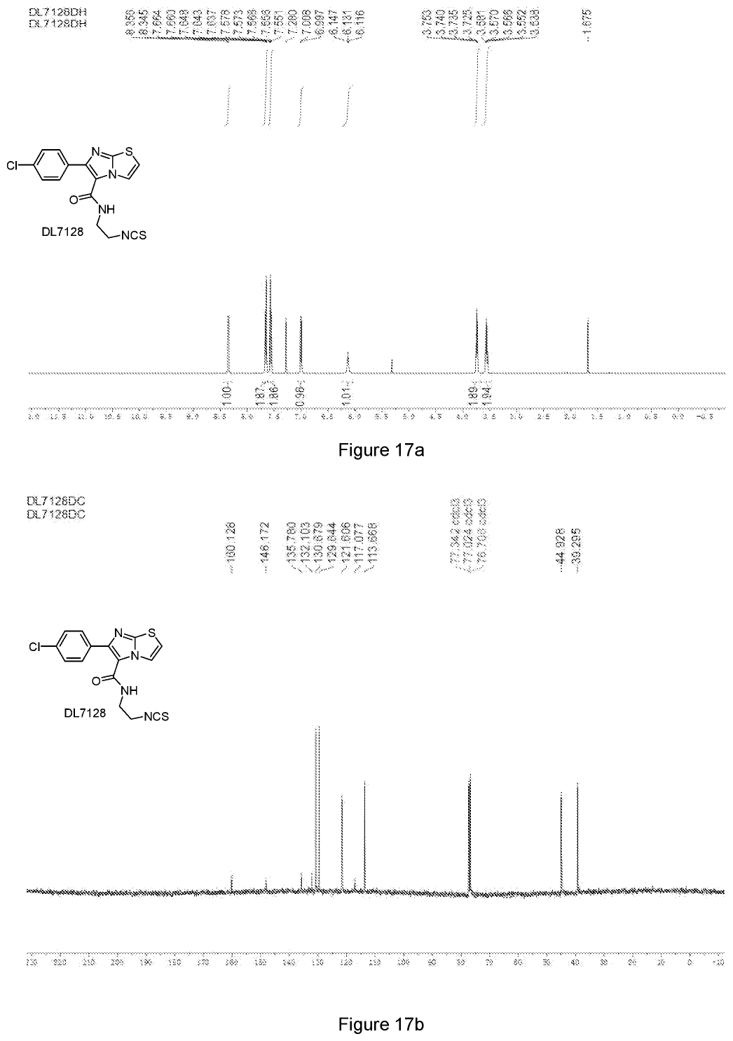

¹H NMR (400 MHz, CDCl₃): δ 8.35 (d, J=4.4 Hz, 1H), 7.65 (d, J=8.0 Hz, 2H), 7.56 (d, J=8.4 Hz, 2H), 7.00 (d, J=4.0 Hz, 1H), 6.13 (br s, 1H), 3.74 (t, J=5.2 Hz, 2H), 3.55 (q, J=5.6 Hz, 2H); ¹³C NMR (100 MHz, CDCl₃): δ 160.1, 148.2, 135.8, 132.1, 130.7, 129.7, 121.6, 117.1, 113.7, 44.9, 39.3. HRMS (ESI): Exact mass calcd for C₁₅H₁₂ClN₄OS₂ [M+H]⁺ 363.0142, found 363.0132. The ¹H NMR spectrum is shown in FIG. 17a and the ¹³C NMR spectrum is shown in FIG. 17b.

### 6-(Naphthalen-2-yl)imidazo[2,1-b]oxazol-5-amine

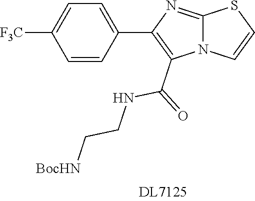 [SMILES: CC(C)(C)OC(=O)NCCNC(=O)c1c(-c2ccc(C(F)(F)F)cc2)nc2sccn12]

¹H NMR (400 MHz, CDCl₃): δ 8.30 (d, J=4.0 Hz, 1H), 7.84 (d, J=7.6 Hz, 2H), 7.76 (d, J=7.6 Hz, 2H), 6.98 (d, J=4.8 Hz, 1H), 6.23 (s, 1H), 4.85 (s, 1H), 3.48-3.44 (m, 2H), 3.27-3.24 (m, 2H), 1.37 (s, 9H); ¹³C NMR (100 MHz, CDCl₃): δ 160.4, 156.3, 152.1, 147.0, 139.8, 137.4, 131.2, 129.6, 125.9, 121.5, 117.9, 113.7, 79.7, 40.5, 39.8, 28.2.

### N-(2-Isothiocyanatoethyl)-6-(4-(trifluoromethyl)phenyl)imidazo[2,1-b]thiazole-5-carboxamide

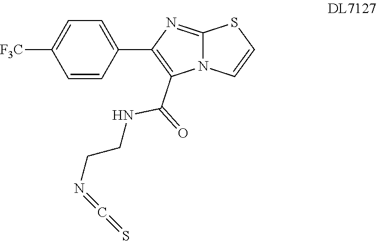 [SMILES: O=C(NCCN=C=S)c1c(-c2ccc(C(F)(F)F)cc2)nc2sccn12]

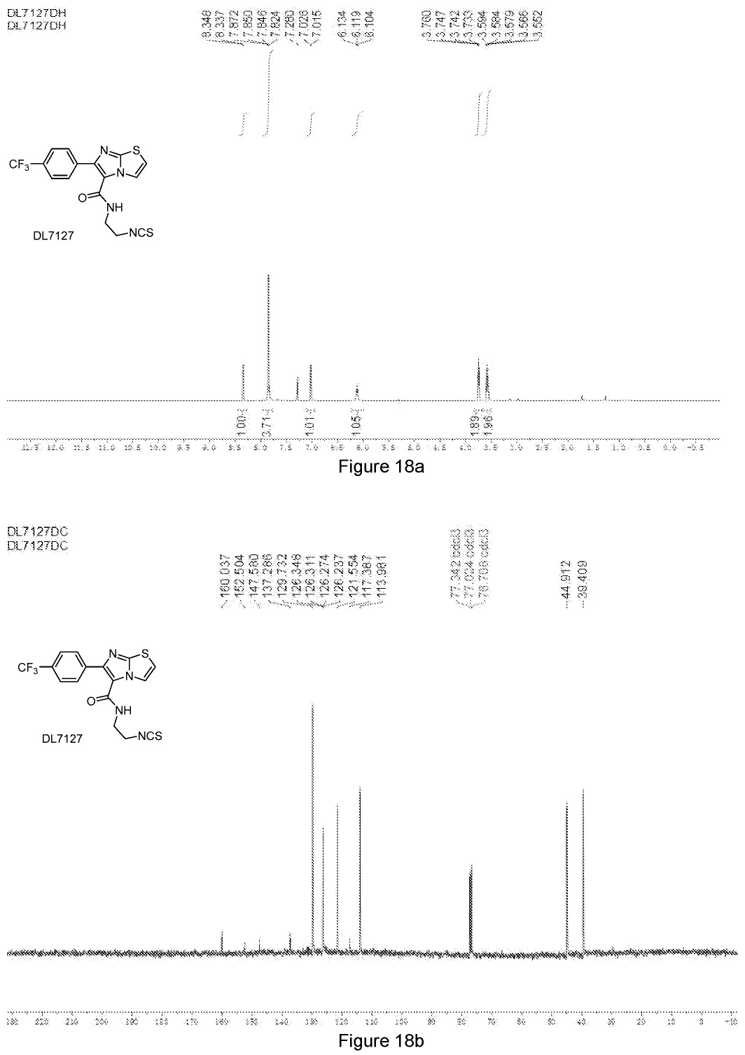

¹H NMR (400 MHz, CDCl₃): δ 8.34 (d, J=4.8 Hz, 1H), 7.87-7.82 (m, 4H), 7.02 (d, J=3.2 Hz, 1H), 6.12 (br s, 1H), 3.75 (t, J=5.2 Hz, 2H), 3.57 (q, J=5.2 Hz, 2H); ¹³C NMR (100 MHz, CDCl₃): δ 160.1, 152.5, 147.6, 139.1, 137.3, 132.9, 131.6, 131.3, 129.7, 126.32, 126.30, 121.6, 117.4, 114.0, 44.9, 39.4. HRMS (ESI): Exact mass calcd for C₁₆H₁₂F₃N₄OS₂ [M+H]⁺ 397.0405, found 397.0403. The ¹H NMR spectrum is shown in FIG. 18a and the ¹³C NMR spectrum is shown in FIG. 18b.

### Tert-butyl (1-(6-(naphthalen-2-yl)imidazo[2,1-b]thiazole-5-carboxamido)propan-2-yl)carbamate

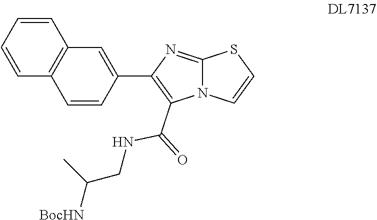 [SMILES: CC(CNC(=O)c1c(-c2ccc3ccccc3c2)nc2sccn12)NC(=O)OC(C)(C)C]

¹H NMR (400 MHz, CDCl₃): δ 8.25 (d, J=4.8 Hz, 1H), 8.14 (s, 1H), 7.93-7.85 (m, 3H), 7.74 (d, J=9.6 Hz, 1H), 7.54-7.51 (m, 2H), 6.88 (d, J=4.4 Hz, 1H), 6.10 (d, J=7.6 Hz, 1H), 5.00 (t, J=5.6 Hz, 1H), 4.21-4.16 (m, 1H), 3.14-3.08 (m, 1H), 3.04-2.98 (m, 1H), 1.32 (s, 9H), 0.97 (d, J=6.4 Hz, 3H).

### N-(2-Isothiocyanatopropyl)-6-(naphthalen-2-yl)imidazo[2,1-b]thiazole-5-carboxamide

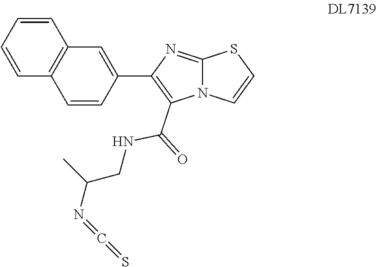 [SMILES: CC(CNC(=O)c1c(-c2ccc3ccccc3c2)nc2sccn12)N=C=S]

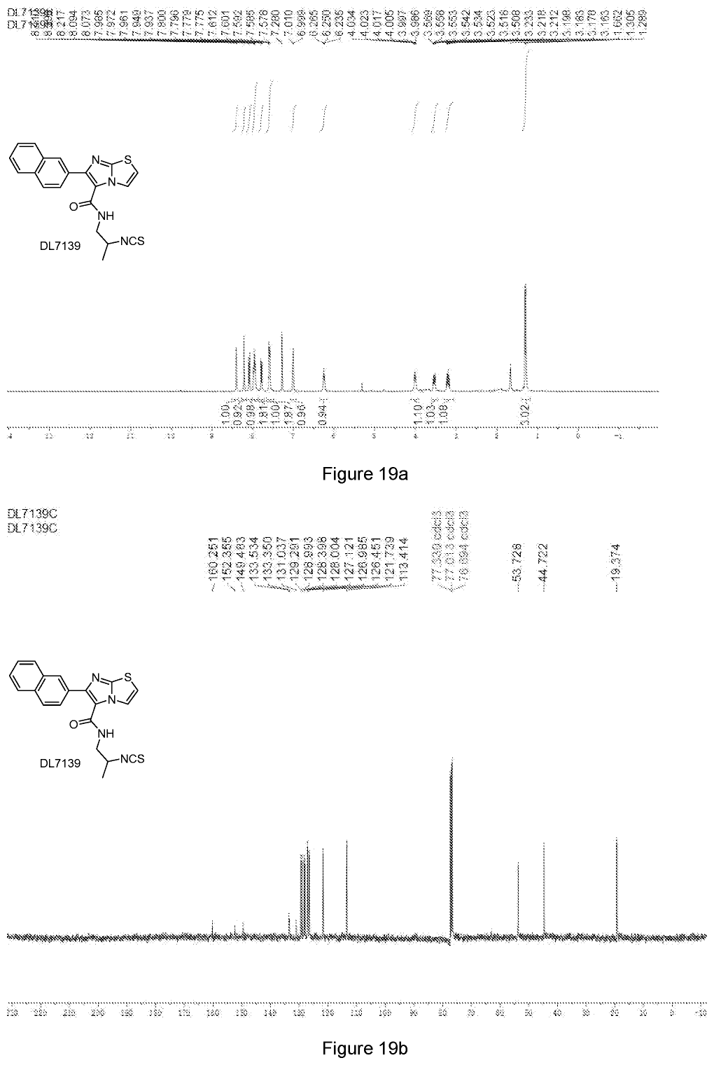

¹H NMR (400 MHz, CDCl₃): δ 8.40 (d, J=4.4 Hz, 1H), 8.22 (s, 1H), 8.08 (d, J=8.0 Hz, 1H), 7.99-7.84 (m, 2H), 7.79 (d, J=8.8 Hz, 1H), 7.60-7.58 (m, 2H), 7.00 (d, J=4.0 Hz, 1H), 6.25 (s, 1H), 4.03-3.99 (m, 1H), 3.57-3.51 (m, 1H), 3.23-3.16 (m, 1H), 1.29 (d, J=7.2 Hz, 3H); ¹³C NMR (100 MHz, CDCl₃): δ 160.3, 152.4, 149.5, 133.5, 133.4, 131.1, 129.3, 129.0, 128.4, 128.0, 127.1, 127.0, 126.5, 121.8, 113.4, 53.7, 44.7, 19.4. HRMS (ESI): Exact mass calcd for C₂₀H₁₇N₄OS₂ [M+H]⁺ 393.0844, found 393.0847. The ¹H NMR spectrum is shown in FIG. 19a and the ¹³C NMR spectrum is shown in FIG. 19b.

### t-Butyl(2-(6-(naphthalen-2-yl)imidazo[2,1-b]thiazole-5-carboxamido)propyl)carbamate

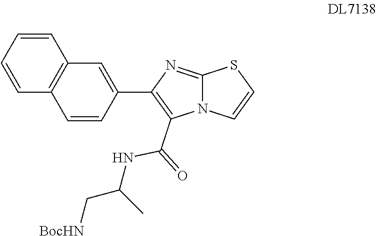 [SMILES: CC(CNC(=O)OC(C)(C)C)NC(=O)c1c(-c2ccc3ccccc3c2)nc2sccn12]

¹H NMR (400 MHz, CDCl₃): δ 8.36 (d, J=4.0 Hz, 1H), 8.19 (s, 1H), 7.99-7.89 (m, 3H), 7.76 (d, J=8.4 Hz, 1H), 7.57-7.54 (m, 2H), 6.96 (d, J=4.0 Hz, 1H), 6.25 (s, 1H), 4.64 (d, J=5.6 Hz, 1H), 3.68 (br s, 1H), 3.40 (br s, 1H), 3.27 (br s, 1H), 1.32 (s, 1H), 1.05 (d, J=6.4 Hz, 3H); ¹³C NMR (100 MHz, CDCl₃): δ 160.7, 155.5, 152.0, 148.8, 133.4, 133.3, 131.2, 128.9, 128.8, 128.3, 127.9, 127.0, 126.9, 126.6, 121.7, 117.6, 113.2, 79.3, 47.0, 44.2, 28.2, 18.6.

### N-(1-Isothiocyanatopropan-2-yl)-6-(naphthalen-2-yl)imidazo[2,1-b]thiazole-5-carboxamide

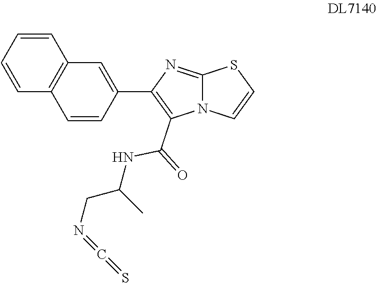 [SMILES: CC(CN=C=S)NC(=O)c1c(-c2ccc3ccccc3c2)nc2sccn12]

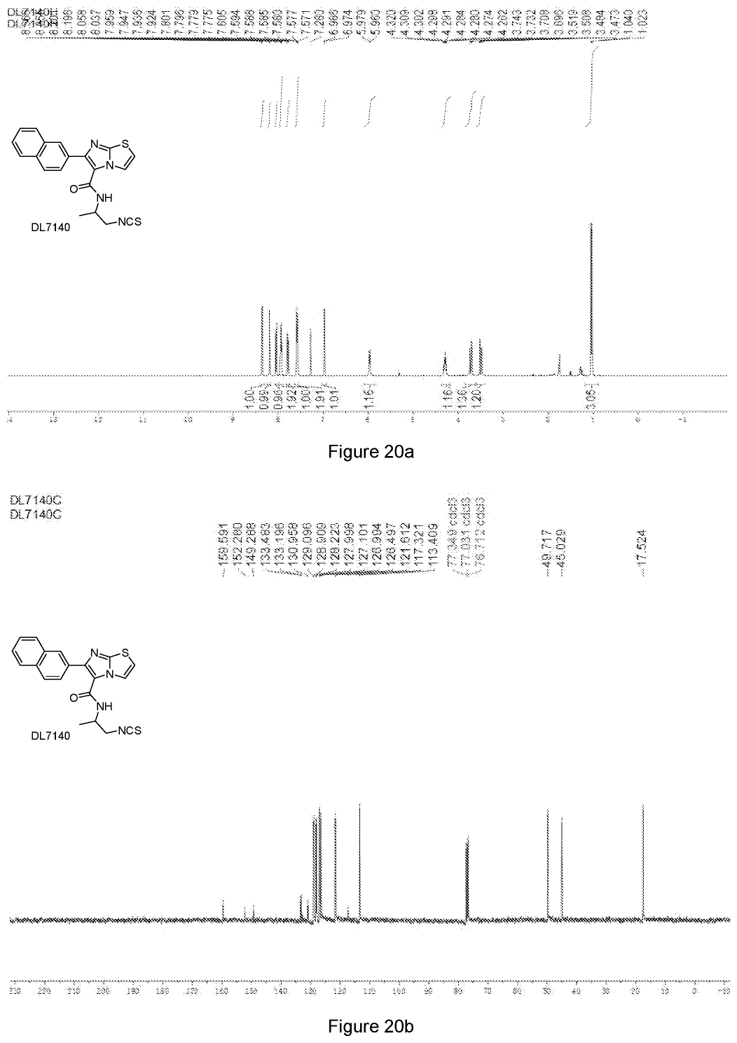

¹H NMR (400 MHz, CDCl₃): δ 8.36 (d, J=4.8 Hz, 1H), 8.20 (s, 1H), 8.05 (d, J=8.8 Hz, 1H), 7.96-7.92 (m, 2H), 7.80-7.78 (m, 1H), 7.60-7.57 (m, 2H), 6.98 (d, J=4.8 Hz, 1H), 5.97 (d, J=6.8 Hz, 1H), 4.32-4.26 (m, 1H), 3.74-3.70 (m, 1H), 3.52-3.47 (m, 1H), 1.03 (d, J=6.8 Hz, 3H); ¹³C NMR (100 MHz, CDCl₃): δ 159.6, 152.3, 149.3, 133.5, 133.2, 131.0. 129.1, 128.9, 128.2, 128.0, 127.1, 127.0, 126.5, 121.6, 117.3, 113.4, 49.7, 45.0, 17.5. HRMS (ESI): Exact mass calcd for C₂₀H₁₇N₄OS₂ [M+H]⁺ 397.0405, found 397.0839. The ¹H NMR spectrum is shown in FIG. 20a and the ¹³C NMR spectrum is shown in FIG. 20b.

### Synthesis and Characterization of DL7021

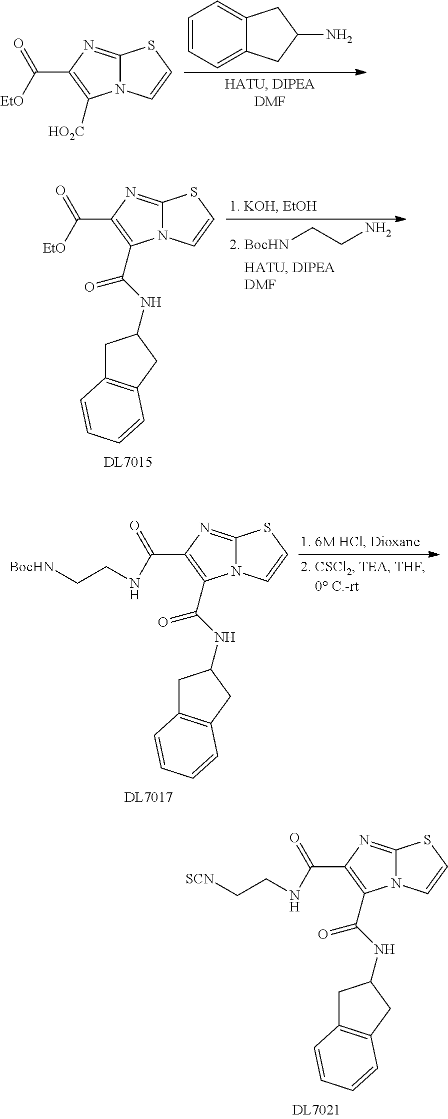

To a solution of 6-(ethoxycarbonyl)imidazo[2,1-b]thiazole-5-carboxylic acid (2 mmol, which was synthesized based on previously published methods: Eur. J Med. Chem. 2019, 179, 84-99, which is incorporated by reference herein in its entirety) in DMF (5 mL) was added HATU (1.2 mmol) and DIPEA (3 mmol), followed by 2,3-dihydro-1H-inden-2-amine (1 mmol). Upon completion of the reaction (followed by TLC), the reaction mixture was partitioned between NaCl (25 mL) and EtOAc (25 mL). The aqueous layer was extracted by EtOAc (25 mL×2). The combined organic layers were dried over Na₂SO₄, and concentrated. The crude material was purified by flash chromatography to give amide compound.

### Ethyl 5-((2,3-dihydro-TH-inden-2-yl)carbamoyl)imidazo[2,1-b]thiazole-6-carboxylate

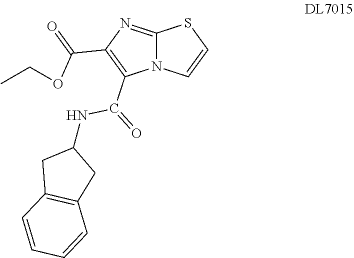 [SMILES: CCOC(=O)c1nc2sccn2c1C(=O)NC1Cc2ccccc2C1]

¹H NMR (400 MHz, CDCl₃): δ10.41 (d, J=6.8 Hz, 1H), 8.64 (d, J=4.8 Hz, 1H), 7.23-7.14 (m, 4H), 7.04 (d, J=4.4 Hz, 1H), 4.86-4.81 (m, 1H), 4.42 (q, J=7.2 Hz, 2H), 3.42 (dd, J=7.6 Hz, J=16 Hz, 2H), 3.01 (dd, J=6.4 Hz, J=16 Hz, 2H), 1.43 (t, J=7.2 Hz, 3H); ¹³C NMR (100 MHz, CDCl₃): δ 165.0, 158.3, 151.0, 140.9, 135.9, 126.7, 125.7, 124.6, 122.3, 116.0, 62.5, 50.8, 39.7, 14.2.

The ester (1.5 mmol) was dissolved in ethanol (5 mL). To this stirred solution potassium hydroxide (12.5 mmol) in water (5 mL) was added. The mixture was then heated under reflux for 3 h and thereafter evaporated to dryness. The residue was dissolved in water (5 mL) and acidified with concentrated hydrochloric acid. The separated solid was collected by filtration and dried without further purification for next step. (See J. Med. Chem. 2003, 46, 3914-3929, which is incorporated by reference herein in its entirety)

To a solution of above acid (1 mmol) in DMF (5 mL) was added HATU (1.2 mmol) and DIPEA (3 mmol), followed by tert-butyl (2-aminoethyl)carbamate (1 mmol). Upon completion of the reaction (followed by TLC), the reaction mixture was partitioned between NaCl (25 mL) and EtOAc (25 mL). The aqueous layer was extracted by EtOAc (25 mL×2). The combined organic layers were dried over Na₂SO₄, and concentrated. The crude material was purified by flash chromatography to give amide compound with Boc protected amine.

### Tert-butyl (2-(5-((2,3-dihydro-1H-inden-2-yl)carbamoyl)imidazo[2,1-b]thiazole-6-carboxamido)ethyl)carbamate

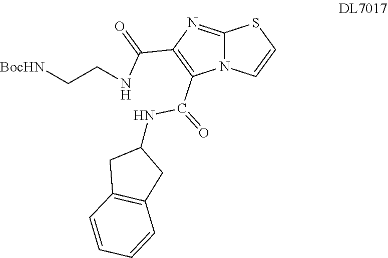 [SMILES: CC(C)(C)OC(=O)NCCNC(=O)c1nc2sccn2c1C(=O)NC1Cc2ccccc2C1]

¹H NMR (400 MHz, CDCl₃): δ 11.4 (d, J=6.4 Hz, 1H), 8.60 (d, J=4.4 Hz, 1H), 8.06 (s, 1H), 7.22-7.13 (m, 3H), 6.95 (d, J=4.8 Hz, 1H), 5.24 (t, J=6.4 Hz, 1H), 4.85-4.76 (m, 1H), 3.52-3.34 (m, 6H), 3.06-3.00 (m, 2H), 1.40 (s, 9H); ¹³C NMR (100 MHz, CDCl₃): δ 163.9, 158.7, 156.2, 149.8, 141.1, 138.7, 126.7, 126.6, 124.5, 122.3, 114.8, 79.5, 50.7, 40.3, 39.9, 39.7, 28.3.

The Boc protected amine (0.5 mmol) was taken up 6M HCl in dioxane at 0° C. and allowed to stir at room temperature for 2 h. The reaction mixture was diluted with EtOAc and concentrated under reduced pressure to afford the TFA salt of amine. To a solution of the TFA salt of amine (0.5 mmol) in 5 mL of THF was added dropwise triethylamine (1.5 mmol) at 0° C. After stirring for 5 min, thiphosgene (0.5 mmol) was added dropwise at 0° C. The resulting solution was slowly warmed to room temperature, stirred for 1.5 h and then cooled to 0° C. The reaction mixture was partitioned between H₂O (25 mL) and EtOAc (25 mL). The aqueous layer was extracted by EtOAc (25 mL×2). The combined organic layers were dried over Na₂SO₄, and concentrated. The crude material was purified by flash chromatography to give final compounds.

### N⁵-(2,3-Dihydro-1H-inden-2-yl)-N⁶-(2-isothiocyanatoethyl)imidazo[2,1-b]thiazole-5,6-dicarboxamide

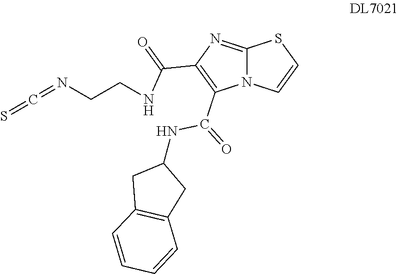 [SMILES: O=C(NCCN=C=S)c1nc2sccn2c1C(=O)NC1Cc2ccccc2C1]

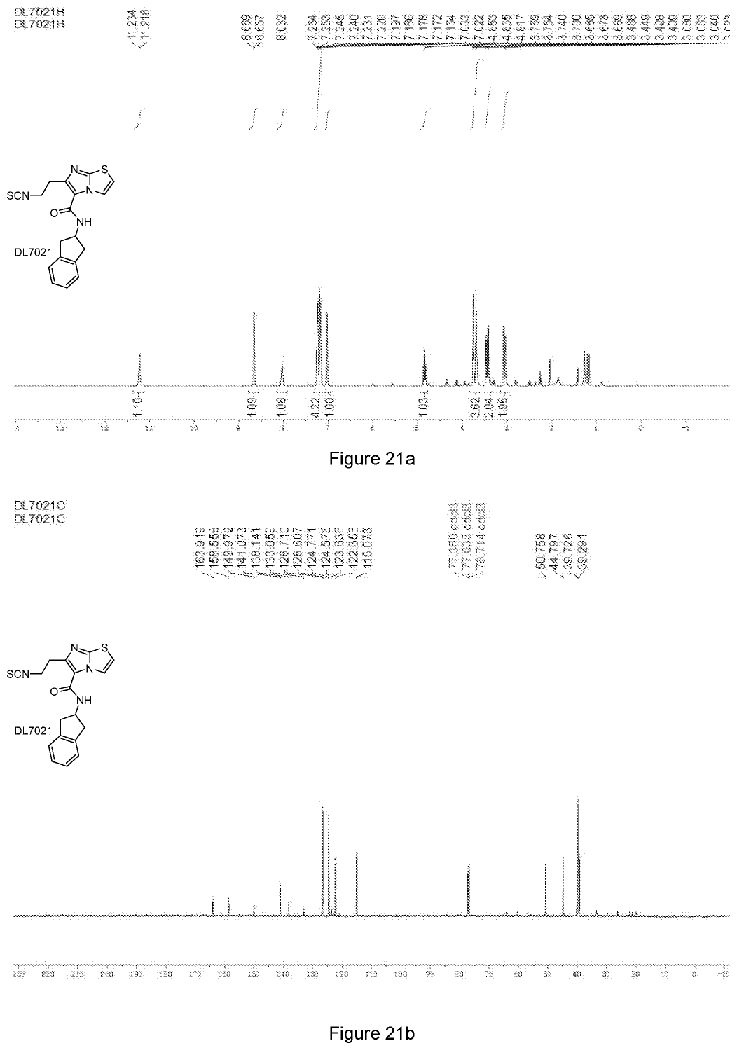

¹H NMR (400 MHz, CDCl₃): δ 11.23 (d, J=6.4 Hz, 1H), 8.66 (d, J=4.8 Hz, 1H), 8.03 (s, 1H), 7.25-7.16 (m, 4H), 7.03 (d, J=4.8 Hz, 1H), 4.87-4.82 (m, 1H), 3.77-3.66 (m, 4H), 3.47-3.39 (m, 2H), 3.08-3.02 (m, 2H); ¹³C NMR (100 MHz, CDCl₃): δ 163.9, 158.6, 150.0, 141.1, 138.2, 133.1, 126.7, 126.6, 124.8, 124.6, 122.4, 115.1, 50.8, 44.8, 39.7, 39.3. HRMS (ESI): Exact mass calcd for C₁₉H₁₈N₅O₂S₂ [M+H]⁺ 412.0902, found 389.412.0911. The ¹H NMR spectrum is shown in FIG. 21a and the ¹³C NMR spectrum is shown in FIG. 21b.

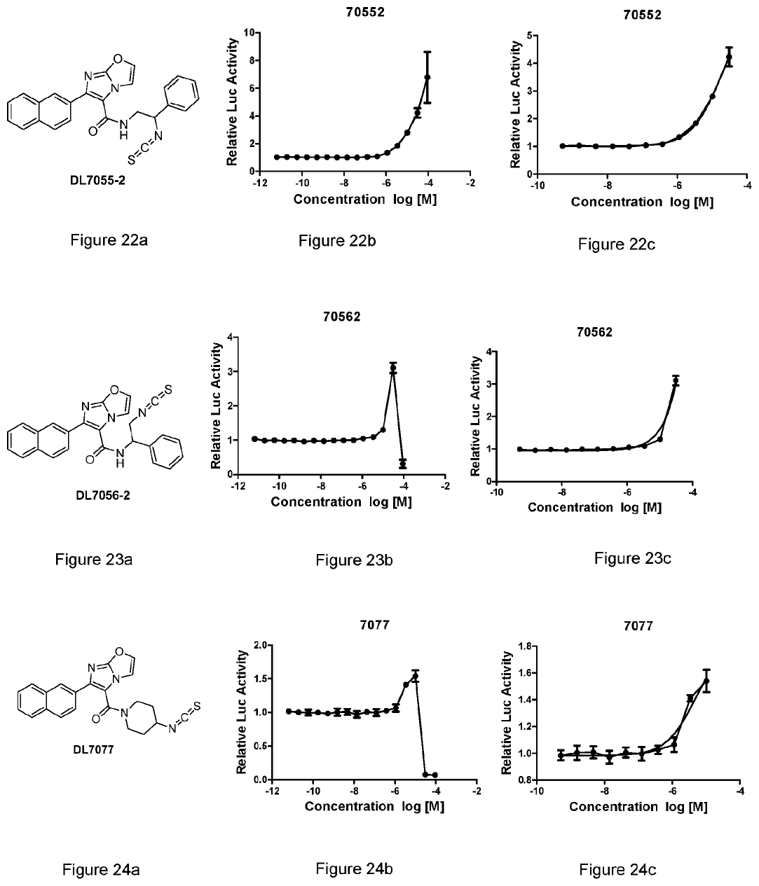

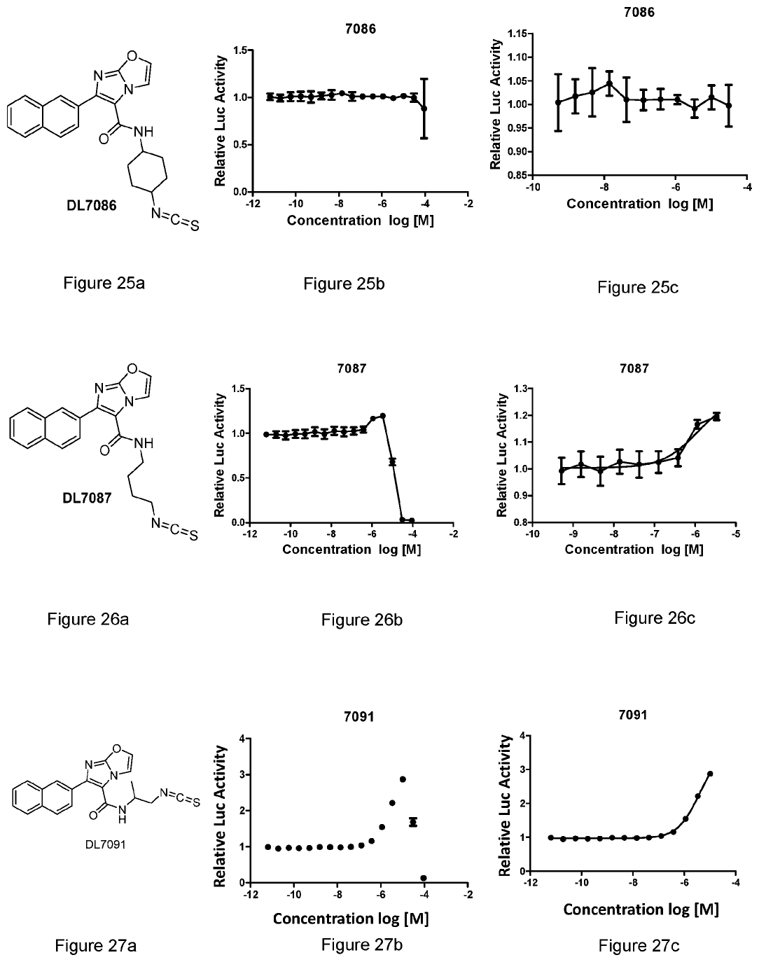

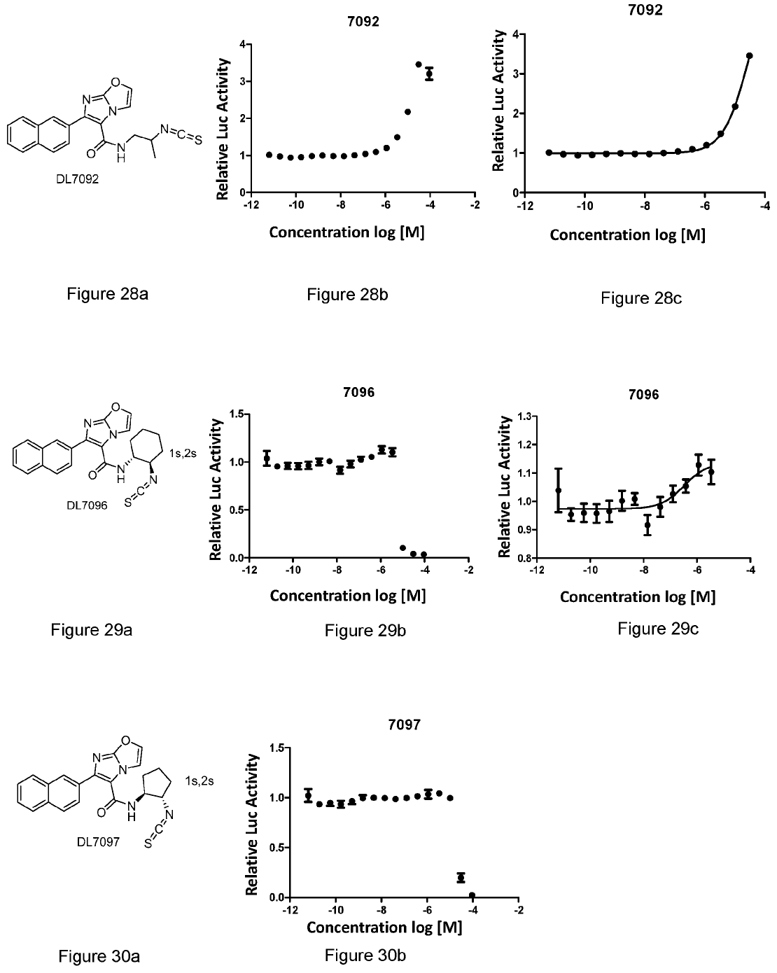

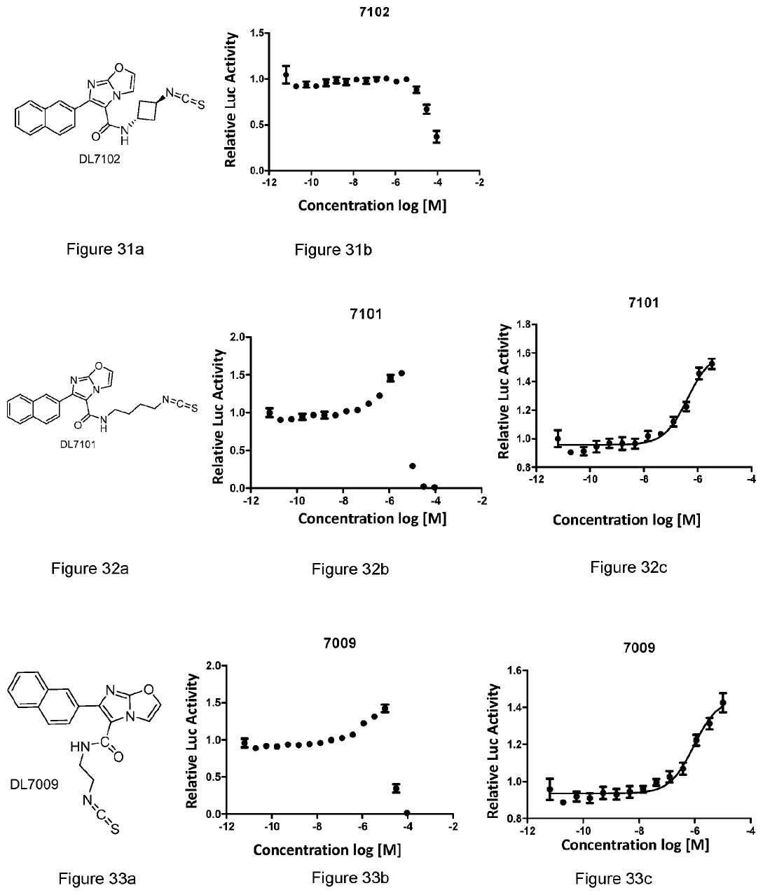

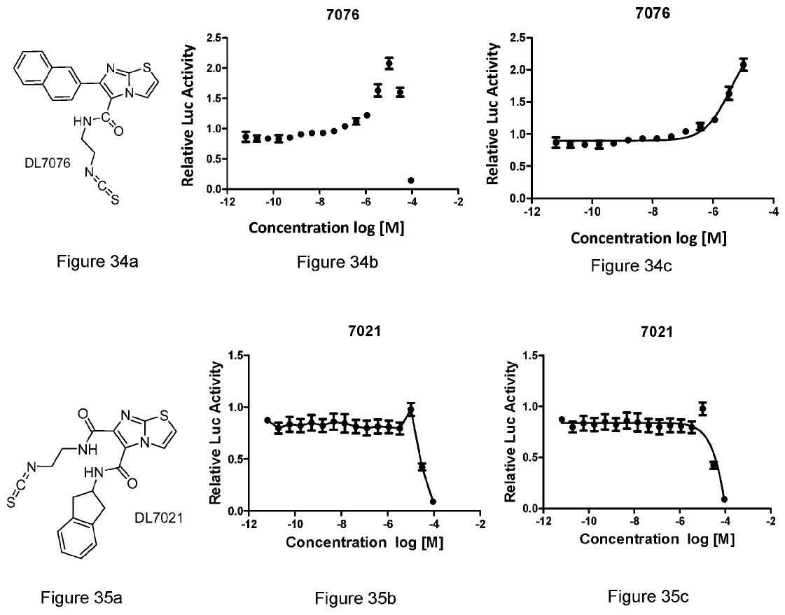

Experimental data demonstrating all curves and EC₅₀ curves of selected compounds can be found in FIG. 22a through FIG. 35c.

### Chemicals and Biological Reagents

CPA, doxorubicin, semicarbazide hydrochloride (SCZ) and 2′,7′-dichlorofluorescein diacetate were obtained from Sigma-Aldrich (St. Louis, Mo.), while dimethyl sulfoxide (DMSO), Phenobarbital (PB), 6-(4-Chlorophenyl)imidazo[2,1-b][1,3]thiazole-5-carbaldehyde-O-(3,4-dichlorobenzyl)oxime (CITCO) were purchased from Millipore Sigma (St. Louis, Mo.). Hybrid compounds were synthesized and kindly provided by Dr. Fengtian Xue (Department of Pharmaceutical Science, University of Maryland). Oligoniclueotide primers were synthesized by Integrated DNA Technologies (Coralville, Iowa). Insulin, ITS+, and Matrigel culture supplies were obtained from BD Biosciences (Bedford, Mass.) (Millipore Sigma).

### Culture of HPH

HPH were obtained from BIOIVT (Baltimore, Md.) and seeded at 1.5×10⁶ or 7.5×10⁵ cells/well in 6- or 12-well collagen I-coated plates, while 24-well collagen I-coated plates were seeded at 3.75×10⁵ cells/well as described previously. 24 hours after seeding, HPH were cultured cells were overlaid with 0.25 mg/mL Matrigel Corning, Corning, N.Y.) 24 h after seeding in serum-free William's E Medium (WEM) supplemented with ITS+(insulin, transferrin, and selenium, 100×; BD Biosciences, Bedford, Mass.), 0.1 μM dexamethasone (DEX; Millipore Sigma), 100 U/ml penicillin, and 100 μg/ml streptomycin (Pen-Strep; ThermoFisher Scientific, Waltham, Mass.), and 2 mM L-glutamine (Invitrogen, Carlsbad, Calif.). Overlaid HPH were treated with compounds in Complete WEM (CYP2B6 and CYP3A4 studies and coculture studies).

### Culture and Treatment of Triple Negative Breast Cancer Cells and Cardiomyocytes

Immortalized triple negative breast cancer cell lines MDA-MB-231 and BT549 were obtained from ATCC (Rockville, Md.). Additionally, the h9c2 cell line was purchased from ATCC (Rockville, Md.). MDA-MB-231 and h9c2 cell lines were cultured in DMEM medium containing 10% fetal bovine serum and 1% penicillin/streptomycin, while the BT549 cell line was cultured in RPMI-1640 containing 10% fetal bovine serum and 1% penicillin/streptomycin. Human iPSC-derived cardiomyocytes were maintained in Mtesr plus and supplemented according to protocol. These cells were treated for 2 hrs or an overnight as indicated. Following a 2 hr pretreatment, cells were co-treated with DOX at indicated concentrations for 6 or 22 hours. Following the overnight treatments, RNA or protein was isolated. HPH were seeded in collagen-coated 24 well plates and pretreated for 24 hrs. BT549 were seeded on inverted coverslips. After a 24 hr pretreatment, hepatocytes were co-administered as indicated and coverslips were inserted. The cell lines were used for less than 60 passages.

### Luciferase Reporter Assays

HepG2 cells were cultured in 24-well plates (1×10⁵ cells/well) and transfected with either a hCAR1+A expression vector (30 ng/well), a CYP2B6-2.2k reporter (60 ng/well) reporter, and pRL-TK (10 ng/well) or a ARE expression vector using X-tremeGENE 9 DNA Transfection Reagent (Sigma-Aldrich). After an overnight transfection, cells were treated with a vehicle control (0.1% DMSO), RIF (10 μM), DL7076 (1 μM, 5 μM, and 10 μM) for 24 hours. Cell lysates were assayed for luciferase activity and normalized against the activity of the Renilla luciferase using a Dual-Luciferase Kit (Promega).

### Ad/EYFP-hCAR Translocation Assay

HPH were seeded on collagen-coated 12-well plates. 24 hours after seeding, HPH were infected with Ad/EYFP-hCAR at a concentration of 5 μL/mL of media and incubated overnight. HPH infected cells were treated with a vehicle control (DMSO 0.01%), a positive control (1 mM PB), and compounds of interest for 8 h. Fluorescence was viewed using the YFP channel (Nikon Eclipse Ti) and quantified by identifying the number of total cells expressing EYFP-hCAR over EYFP-hCAR present in the nucleus. Representative images are shown, and quantitative data represents the mean±SD of 3 individual images for each treatment group.

### BT549 Hepatocyte Co-Culture

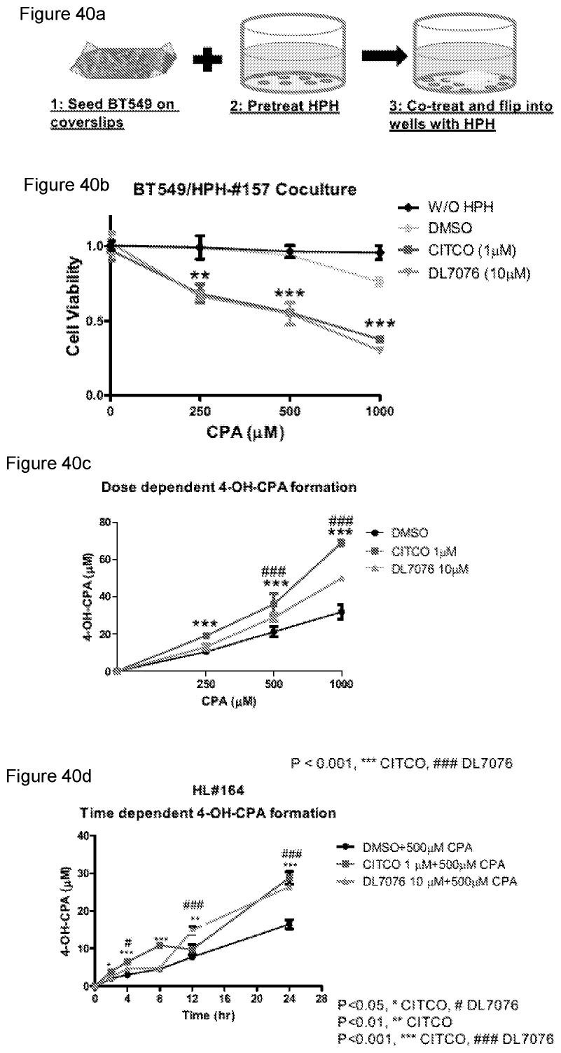

HPH were cultured in collagen-coated 24 well plates and pre-treated with vehicle control (0.1% DMSO), CITCO (1 μM) or DL7076 (5 μM or 10 μM) for 24 h. During the pre-treatment time, BT549 cells were seeded at 0.8×10⁵ on collagen-coated coverslips modified to have their corners bent (FIG. 40a). Following the 24 h pre-treatment, coverslips were combined in hepatocyte seeded wells and exposed to designated concentrations of cyclophosphamide in the presence or absence of DL7076 for various time intervals, as indicated.

### Multi-Organ Co-Culture System

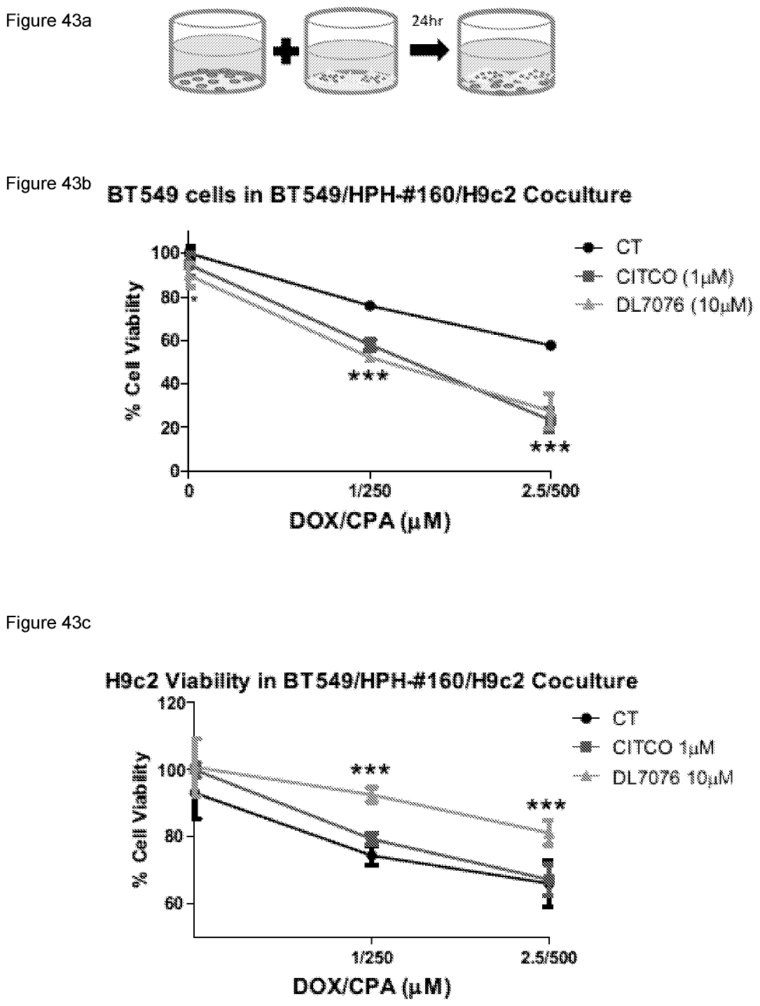

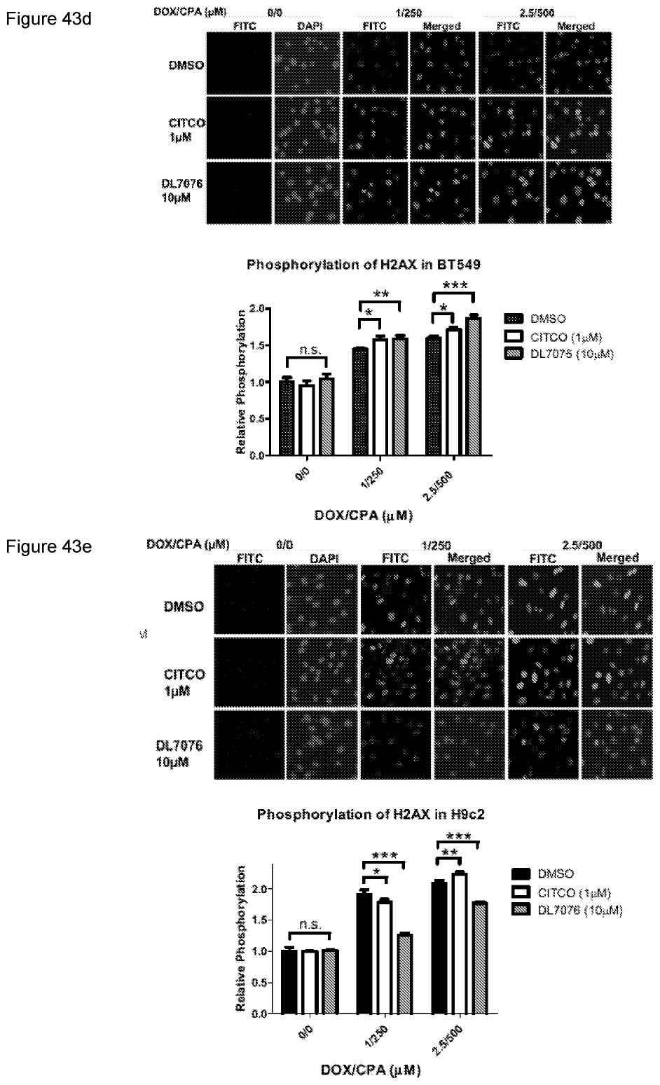

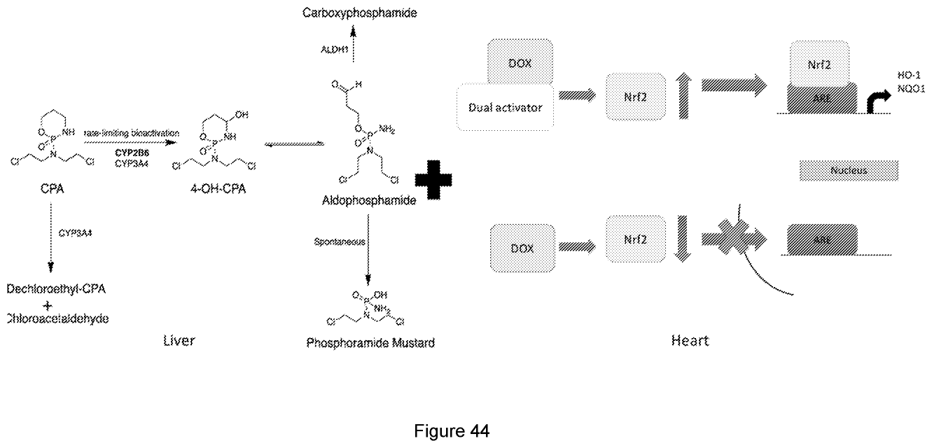

Additionally, HPH were cultured in collagen-coated 6-well plates and pre-treated as described in the previous co-culture system. BT549 and H9c2 cells were plated on similar modified coverslips 0.8×10⁵ cells and allowed to attach for 24 hours during hepatocyte pre-treatment. Following attachment, coverslips were pre-treated for 2 h with vehicle control (0.1% DMSO), CITCO (1 μM), or DL7076 (10 μM). Following pre-treatment, BT549 coverslips were inserted adjacent to H9c2 coverslips over the sandwich cultured HPH as depicted in FIG. 43a. The cultures were exposed to indicated concentrations of cyclophosphamide and doxorubicin in the presence or absence of DL7076 (10 μM). Both BT549 and H9c2 cells were harvested at indicated time points and assess for viability, apoptosis, and DNA damage as detailed further.

### RT-PCR

After 24 h treatment, HPH, BT549, MDA-MB-231, and H9c2 cells were washed and a phenol-chloroform extraction was used to isolate total RNA with the TRIzol reagent (ThermoFisher, Rockford, Ill.). Reverse transcription was performed on extracted RNA (1 μg) to produce cDNA using the High Capacity cDNA Archive kit according to manufacturer's instructions (Applied Biosystems, Foster, Calif.). PCR was performed on an ABI StepOnePlus Real-Time PCR system (Applied Biosystems) using Fast SYBR Green Master Mix (Thermofisher, Rockford, Ill.) to determine gene expression changes. Primer sequences for CYP2B6, CYP3A4, HO-1, and glyceraldehyde-3-phosphate dehydrogenase (GAPDH) are: CYP2B6, 5′-AGACGCCTTCAATCCTGACC-3′ (SEQ ID NO: 1) and 5′-CCTTCACCAAGACAAATCCGC-3′ (SEQ ID NO: 2); CYP3A4, 5′-GTGGGGCTTTTATGATGGTCA-3′ (SEQ ID NO: 3) and 5′-GCCTCAGATTTCTCACCAACACA-3′ (SEQ ID NO: 4); HO-1 5′-GGGTGATAGAAGAGGCCAAGACT-3′ (SEQ ID NO: 5) and 5′-AGCTCCTGCAACTCCTCAAAG-3′ (SEQ ID NO: 6) GAPDH, 5′-CCCATCACCATCTTCCAGGAG-3′ (SEQ ID NO: 7) and 5′-GTTGTCATGGATGACCTTGGC-3′ (SEQ ID NO: 8). Quantification of expression changes was done by the 2^{ΔΔCt} method, normalizing to GAPDH as the housekeeping gene and determining the fold change compared to control values (n=3).

### Western Blot Analysis

Protein was harvested using cell lysis buffer (Cell Signaling Technology, Danvers, Mass.) with a complete protease inhibitor cocktail (Roche, Mannheim, Germany) prior to homogenizing via sonification. Homogenized protein from treated HPH, H9c2, BT549, and MDA-MB-231 cells were resolved on NuPage Novex Bis-Tris 4-12% gels (Life Technologies) or NuPage Novex Bis-Tris 12% gel and transferred through electrophoresis onto immobilon-P polyvinylidene difluoride (PVDF) membranes. Membranes were blocked with 5% BSA or 5% milk in Tris-Buffer saline with Tween 20 (TBST). Further, membranes were incubated with specific antibodies against human CYP2B6 (1:1000, Abcam), CYP3A4 (1:1000, Millipore Sigma), HO-1 (1:1000, Abcam), cleaved caspase-3 (1:1000 Cell Signaling Technologies), Nrf2 (1:1000, Abcam), TATA-Binding Protein (1:500, Santa Cruz) β-actin (1:3000, Millipore Sigma) at 4° C. overnight. Following overnight incubation, horseradish peroxidase goat anti-mouse or anti-rabbit IgG antibodies were used for secondary incubation. Membranes were developed using SuperSignal West Pico Chemiluminescent Substrate (Thermo Scientific) or Radiance Q (Azure). Two TNBC cell lines, cardiomyocytes, and HPH protein samples were isolated and resolved on a 4-12% or 12% SDS PAGE gel followed by electrophoretic transfer to a PVDF membrane. Membranes were probed 1:1000 for CYP2B6, CYP3A4, Nrf2, cleaved caspase 3, and HO-1 using β-actin (1:3000) or TBP (1:500) as a loading control.

### Cell Viability Assays

H9c2 cells were seeded 0.8×10⁵ in 24-well plates. Following cell attachment, a 2 h pre-treatment with vehicle control (0.1% DMSO), SFN (2.5 μM) or DL7076 (1 μM, 5 μM, or 10 μM) was done followed by doxorubicin (2.5 μM) exposure for 22 h. Co-culture cells were assembled and treated with cyclophosphamide and doxorubicin as indicated. A Cell Counting Kit-8 (Enzo) assay was carried out. Cell viability was described as a percent of vehicle control (0.1% DMSO). Cardiomyocyte, human iPSC-derived cardiomyocytes, and BT549 viability was determined with a 1 hr incubation of 6% CCK8 in WEM.

### LC-MS/MS Determination of 4-OH-CPA Formation

200 μL of cell culture medium was removed from each treatment group was immediately mixed with 20 μL of semicarbazide (SCZ; 2M) to stabilize 4-OH-CPA to its derivative 4-OH-CPA-SCZ. Once removed, samples were vortexed and left at room temperature for 10 minutes. 2 μM HMP was added as an internal standard and 200 μL acetonitrile was used to precipitate protein. Mass transitions were monitored at m/z: 261-140 for CPA, m/z: 334-221 for 4-OH-CPA-SCZ, and m/z: 180-135 for HMP under the positive multiple reaction monitoring mode.

### Oxidative Stress Imaging

H9c2 rat cardiomyocytes seeded at 0.8×10⁵ in 24-well plates. After allowing 24 h for cell attachment, H9c2 cells were pre-treated with vehicle control (0.1% DMSO), SFN (2.5 μM), or DL7076 (5 μM) for 2 h prior to a 6 h exposure to doxorubicin (1 μM, 2.5 μM, and 5 μM). Cells were washed 3× with PBS. Subsequently, 0.5 mL of WEM containing 20 μM 2′-7′-dichlorofluorescein diacetate (DFDA) was added to each well. The cells were incubated at 37° C. and 5% CO₂ for 30 min. The oxidation of DFDA to the fluorescent dichlorofluorescein was viewed using fluorescence microscopy (Nikon Eclipse Ti) with a FITC filter at 900 nm and quantified using Brightspot detection for fluorescent detection of triplicate images and normalized to the vehicle control (0.1% DMSO). Cells on coverslips from co-culture experiments were fixed using 4% paraformaldehyde and permealized using Triton-X 100. A primary antibody for mouse anti-phospho-H2AX (SER139) was incubated overnight. Goat anti-mouse IgG Fluor Alexa 488 (1:200) was incubated for 2 hrs. Phosphorylation was calculated through fluorescence intensity all normalized to the vehicle control.

### Phosphorylated H2AX Imaging

The H9c2 cells were plated on modified coverslips and removed from the multi-organ co-culture system after 24 hrs of combination treatment of cyclophosphamide and doxorubicin in the presence of a vehicle control (0.1% DMSO), CITCO, or DL7076 as described. Cells were fixed using 4% paraformaldehyde in PBS for 15 minutes followed by permeabilization with 0.5% Triton-X 100 in PBS for an additional 15 minutes at room temperature. Prior to blocking with 5% Bovine Serum Albumin (BSA), 3 washes with PBS were done to remove excess Triton-X. Primary antibody to mouse anti-phospho-H2AX (SER139, clone JBW301, Millipore, 1:500 dilution) was prepared in PBS with 3% BSA and 0.3% Triton-X 100 and incubated overnight. Following washing 3× with PBS, cells were incubated at room temperature for 2 hrs with secondary antibody goat-anti-mouse IgG Alexa Fluor 488 (1:200 dilution). Nuclear DNA was stained using DAPI at 100 ng/ml. FITC channel was displayed as red to highlight the phosphorylation. The coverslips were visualized at 10× magnification using fluorescence microscopy (Nikon Eclipse Ti) and the amount of phosphorylation was quantified as mean intensity using brightspot detection of quadruplet images of the whole well and normalized to the vehicle control (0.1% DMSO).

### Statistical Analysis

All statistical analysis were preformed using GraphPad Prism's (v.5) One-Way Analysis of Variance (ANOVA utilizing post-hoc Dunnett's or Bonferroni analysis or Two-Way Analysis of Variance (ANOVA) utilizing post-hoc Bonferroni analysis. The mean of measurements ±S.D. are presented unless otherwise noted. Significance was determined at P<0.05 (*), P<0.01 (**), P<0.001 (***).

### Chemistry

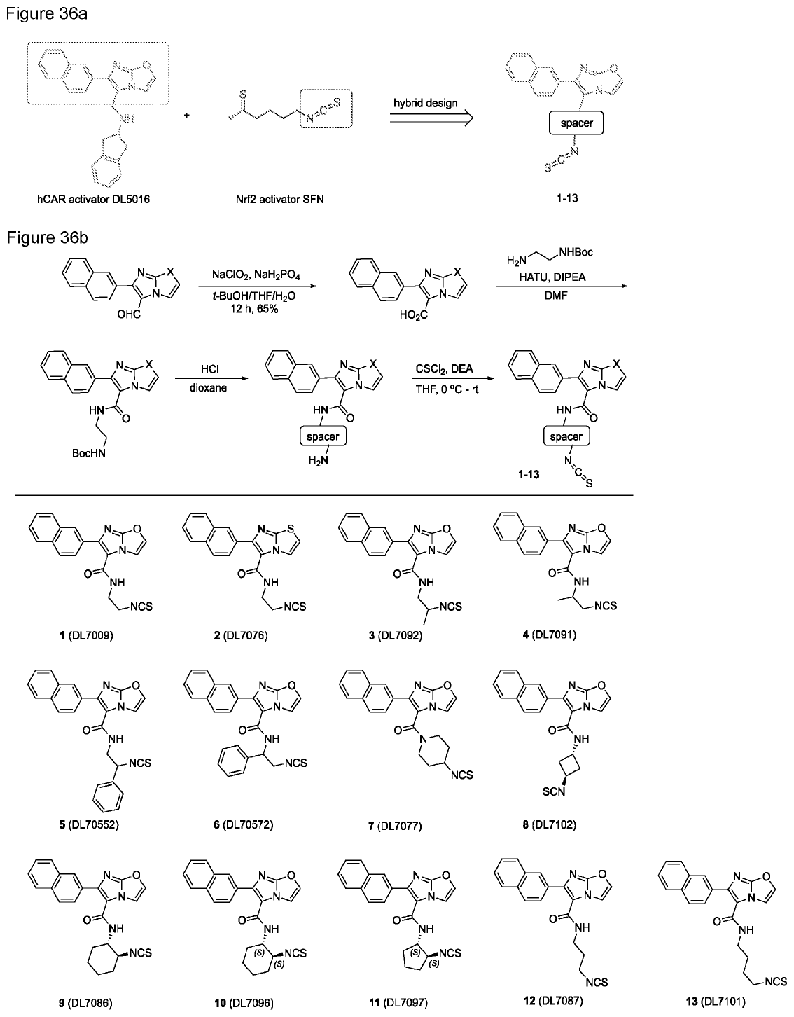

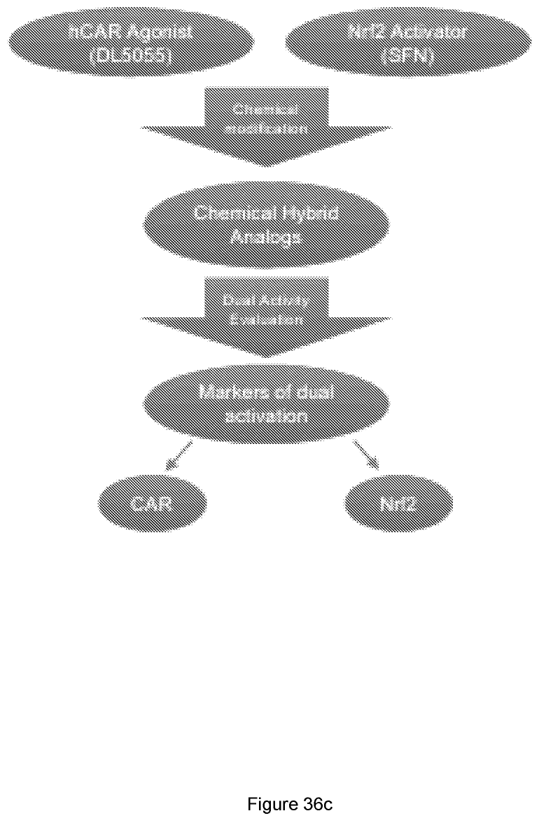

The syntheses of the dual activator compounds 1-13 are summarized in FIGS. 36a-36c. The synthesis of the imidazolethiazole or imidazoleoxazole core of DL5055 and the isothiocyanate group of the sulforaphane of compounds 1-13 is described in FIG. 36b.

### Results

### Structure-Activity Relationship (SAR)

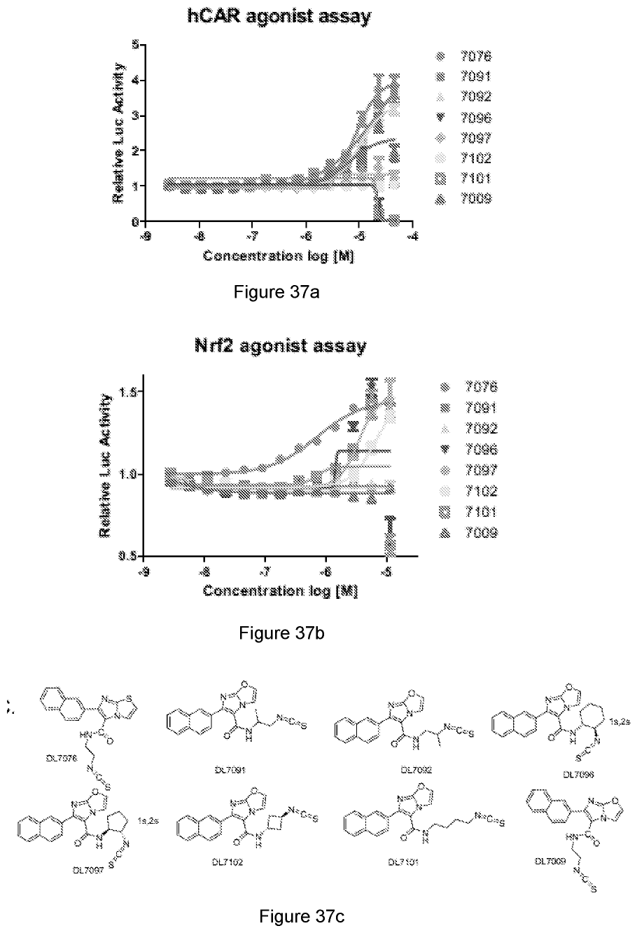

Through the chemical modification of various linkers and the imidazolethiazole or imidazoleoxazole core, the dual activation of these compounds was demonstrated using a hCAR or Nrf2 luciferase gene reporter assay (FIGS. 37a-37c). In order to identify a compound with dual activity, CAR activity was shown in FIG. 37a, where DL7076 was shown to have the highest efficacy. Therefore, moving forward to the Nrf2 luciferase assay in FIG. 37b, DL7076 was shown to have higher potency and efficacy when activating Nrf2. Compound 2, DL7076, was selected for further evaluation based on its effects in activating hCAR and Nrf2 using the HepG2 cell reporter assay (FIGS. 37a-37c).

DL7076 as a hCAR Activator and Inducer of Hepatic Expression of CYP2B6

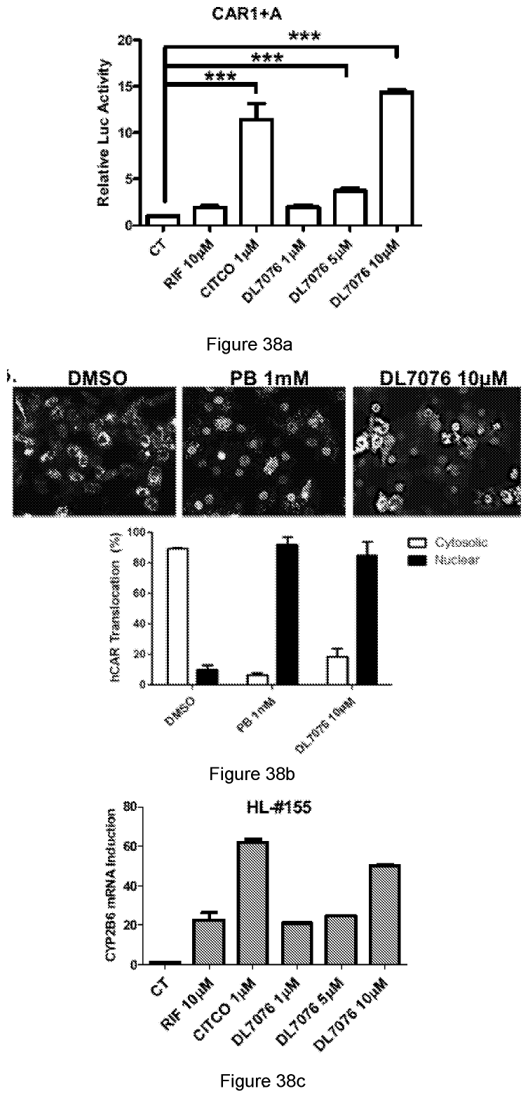

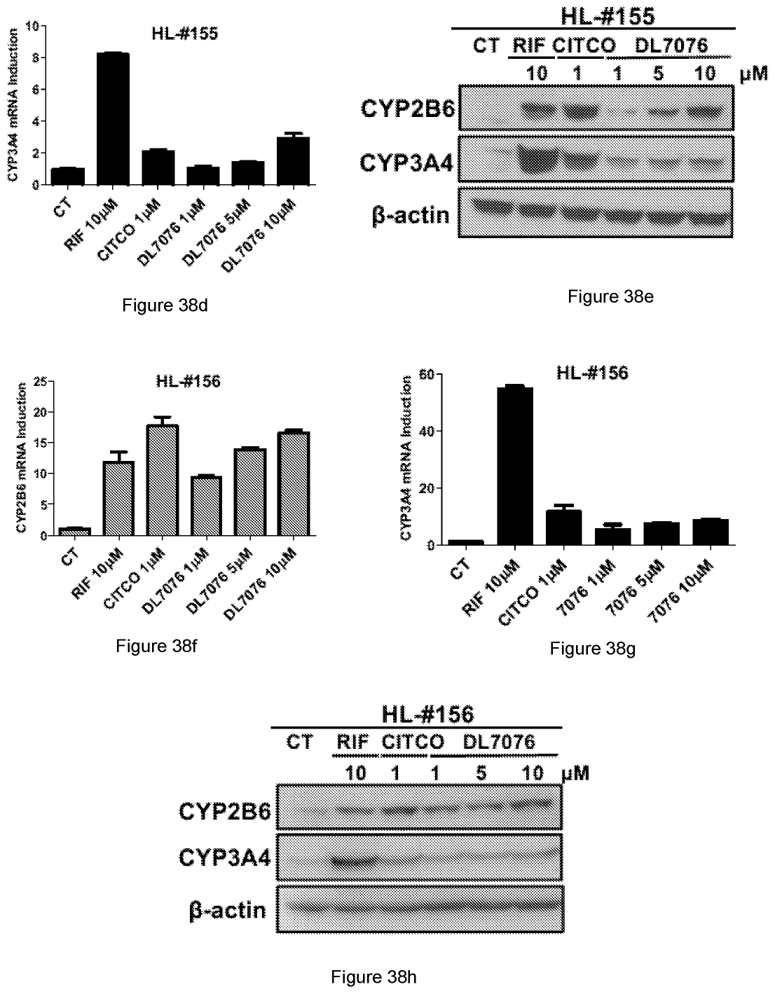

To further confirm compound DL7076 activation of hCAR, CAR activity was evaluated further using a HepG2 luciferase reporter assay, which demonstrated that DL7076 at 10 μM could activate CAR1+A in a comparable manner to the prototypical CAR activator, CITCO (FIG. 38a). Additionally, it has been well-established that CAR activation requires hCAR to translocate from the cytoplasm to the nucleus in HPH as well as in vivo. As shown in FIG. 38b, when administering vehicle control (0.1% DMSO), phenobarbital (PB), or DL7076, DL7076 triggered hCAR nuclear translocation using Ad-EYFP-hCAR-infected HPH. Efficient Ad-EYFP-hCAR translocation into the nucleus can be seen upon treatment with the positive control, PB, and with compound DL7076, while the vehicle control-treated HPH mostly remains in the cytoplasm. Without being bound by theory, this result indicates that DL7076 can be shown to mediate the crucial translocation steps in hCAR activation.

To investigate the effects of DL7076 on the expression of the major drug-metabolizing enzymes responsible for CPA biotransformation, HPH were treated with a positive control (CITCO), DL7076, and a known hPXR activator, Rifampin (RIF) (FIGS. 38c-38h). In two different preparations of human hepatocytes, DL7076 showed a concentration dependent increase in the induction in the expression of CYP2B6 mRNA, whereas minor modulation of the induction of CYP3A4, respectively (FIGS. 38c-38h). The clear preferential induction of CYP2B6 by DL7076 was further confirmed at the protein level in HPH prepared from the same 2 donors. Without being bound by theory, these results indicate that selective activation of hCAR by DL7076 led to robust selective induction of CYP2B6, favoring the biotransformation of CPA.

### DL7076 Modulates Nrf2 Activation Promoting Heme Oxygenase-1

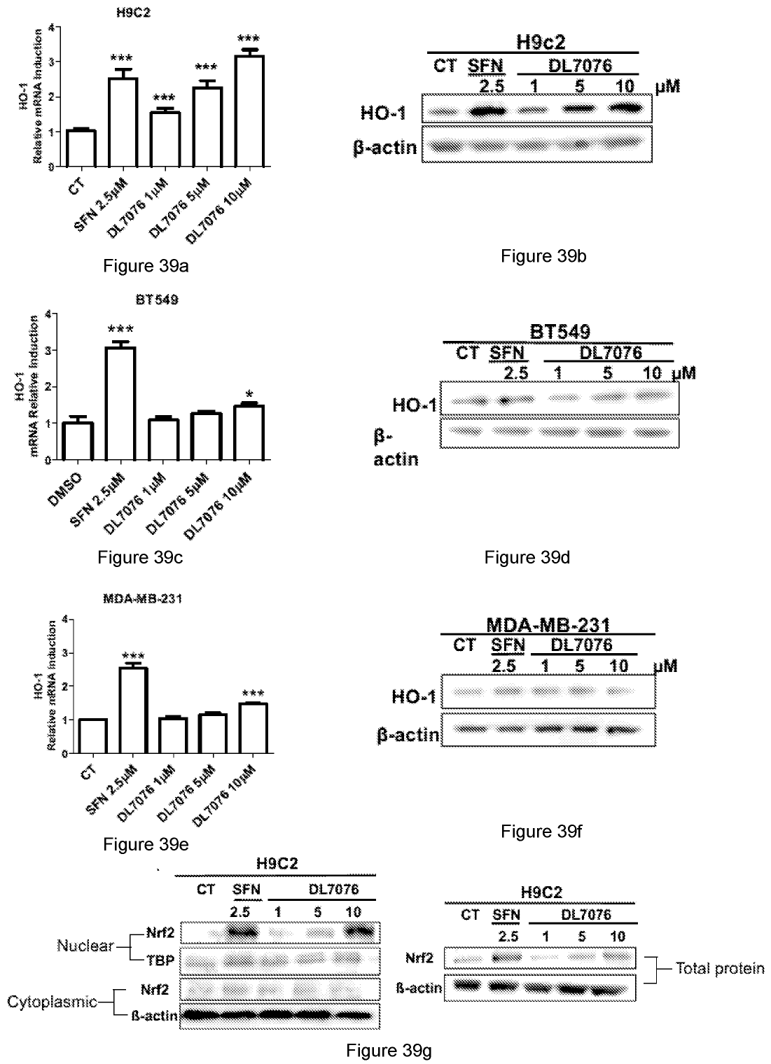

Following the examination of hCAR activation, the effects of DL7076 on the expression of the major Nrf2 target gene, heme oxygenase-1 (HO-1), was investigated. It has been previously demonstrated that HO-1 induction can be cardioprotective, through the reduction of reactive oxygen species. H9c2 cardiomyocytes and IPS-derived cardiomyocytes were treated with a vehicle control (0.1% DMSO), a positive control (SFN), and DL7076 in 3 concentrations (FIG. 39a). In H9c2 cardiomyocytes, DL7076 showed a concentration dependent induction in the expression of HO-1 mRNA, with 10 μM DL7076 producing 3- to 3.5-fold. This robust induction in the expression of HO-1 was confirmed at a protein level. Further, in order to show that DL7076 does not induce the expression of HO-1, leading to a protective effect, in triple negative breast cancer (TNBC) cells, two cell lines were used, BT549 and MDA-MB-231. As shown in FIGS. 39c-39f, both BT549 and MDA-MB-231 have minimal induction of Nrf2 target gene, HO-1, with 10 μM of DL7076 inducing 1.5-fold and 1.4-fold, respectively. Without being bound by theory, these results indicate a tissue specific induction of HO-1, favoring cardioprotection, in the H9c2 cardiomyocytes, but not in the TNBC cells. Whole H9c2 cell lysates were evaluated, demonstrating DL7076 increased Nrf2 expression in a concentration dependent manner (FIG. 39g). IPS-derived cardiomyocytes were used to further validate the induction in the expression of HO-1 by DL7076 in a human primary cell model. To determine DL7076 effect on the first essential step of Nrf2 activation, Nrf2 translocation was demonstrated in nuclear extracts of H9c2 cardiomyocytes. Nrf2 is shown accumulating in a concentration dependent manner within the nucleus, while the cytoplasmic lysates remain stagnant due to both the upregulation of Nrf2 expression and the translocation occurring.

### DL7076 Promotes CPA-Mediated Cytotoxicity of BT549 Cells in BT549/HPH Co-Culture System

The HPH/BT549 cell co-culture system has proven to be an efficient technique to measure efficacy in vitro while also mimicking in vivo conditions of the human body, allowing studies of hepatic metabolism, drug-drug interaction, and extrahepatic effects such as, anti-cancer effect and side toxicities to be considered in a simplified fashion. The HPH/BT549 co-culture system is depicted in FIG. 40a As shown in previously, 10 μM of DL7076 produces substantial hCAR activation and subsequent CYP2B6 induction in HPH, while also producing optimal HO-1 induction; therefore, this concentration was chosen for further studies. HPH were preincubated for 24 hours with a vehicle control (0.1% DMSO), CITCO (1 μM), or DL7076 (10 μM), allowing for gene induction to occur. Multiple pharmacologically relevant concentrations of CPA were co-administered to the coculture with the vehicle control, CITCO, or DL7076 circulating in medium. Without being bound by theory, results from the cocultures indicate that CPA decreased the viability of the BT549 cells in a concentration-dependent manner (FIG. 40b).

Markedly, cotreating CPA with DL7076 significantly enhanced the cytotoxicity of CPA in the BT549 cells. This result is represented in that 250 μM of CPA cotreated with DL7076 could achieve notably increased anti-cancer effect than CPA alone, even at 1000 μM. To further evaluate the effects of DL7076 on the CAR-mediated biotransformation of CPA in HPH/BT549 coculture system, culture medium from each treatment condition was collected and LC-MS/MS quantification of 4-OH-CPA. As shown in FIGS. 40c and 40d. formation of 4-OH-CPA was increased in a concentration and time-dependent manner, respectively, while DL7076 led to the increased rate and extent of CPA biotransformed. Additionally, at a fixed CPA concentration (500 μM), DL7076 enhanced 4-OH-CPA formation in the coculture medium at all time points, at time points ranging from 2 to 24 hours. Without being bound by theory, these results indicate that CPA-mediated cytotoxicity of TNBC cells can be enhanced through CAR-mediated amplification of hepatic 4-OH-CPA formation.

### DL7076 Selectively Recovers H9c2 Cardiomyocyte Viability, but not TNBC

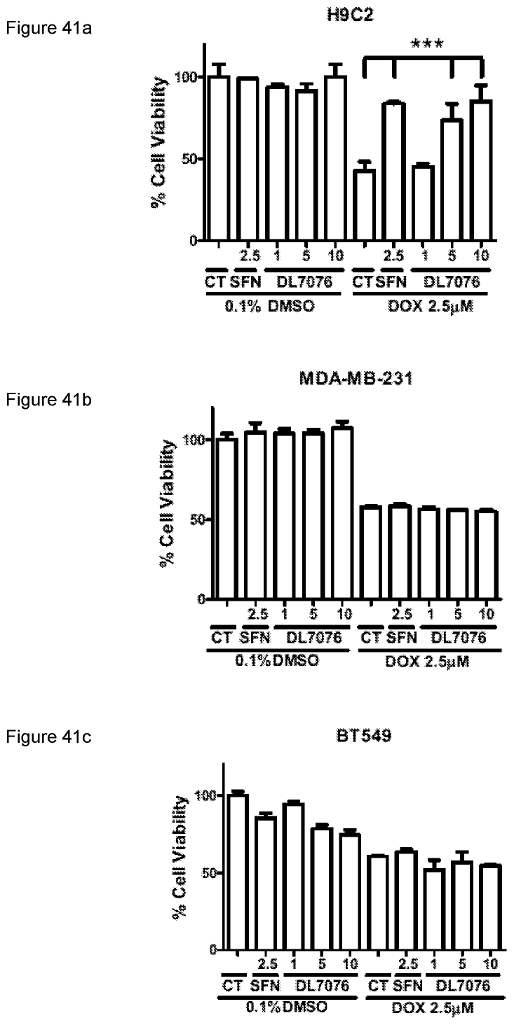

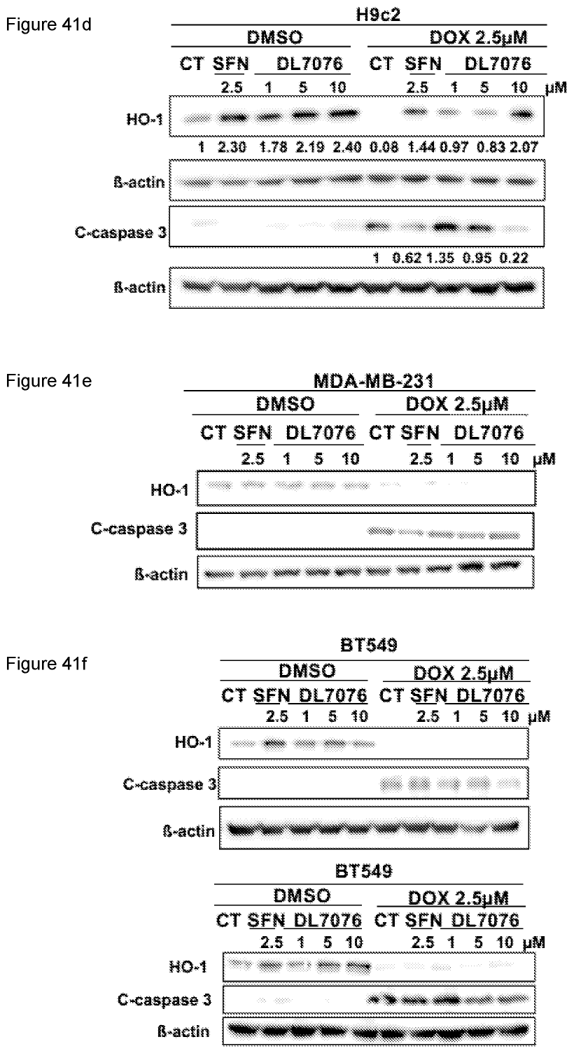

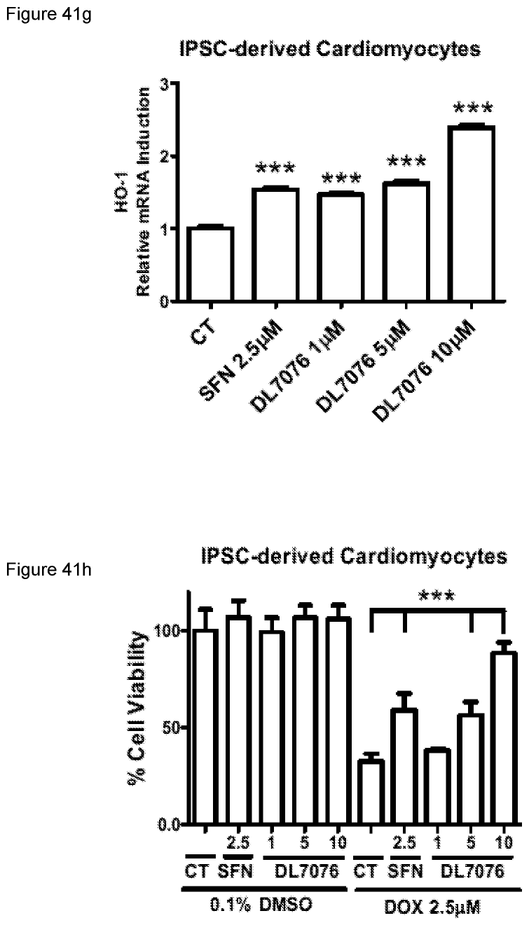

To investigate the protective effects of Nrf2 activation against DOX-induced cardiotoxicity, h9c2 cardiomyocytes were treated with a vehicle control (0.1% DMSO), SFN, and DL7076 at 3 concentrations for 2 hours followed by a 22-hour cotreatment with 2.5 μM doxorubicin (DOX). Without DOX, there was no cytotoxicity seen from DL7076; however, when DOX was administered, there was a concentration dependent reversal in the DOX-induced cardiotoxicity in h9c2 cardiomyocytes (FIG. 41a). Notably, this efficient reversal was not shown in two TNBC cell lines, MDA-MB-231 (FIG. 41b) and BT549 (FIG. 41c), likely due to the lack of HO-1 induction. The mechanism of the reversal in the cardiotoxicity in the h9c2 cardiomyocytes can be confirmed further in FIG. 41d. It was found that DL7076 induces the expression of HO-1, which serves as a defense against ROS, which upon DOX administration, is repressed. However, when there is a pretreatment with DL7076, there was a concentration dependent protection in the expression of HO-1, lending itself to the same trend as the viability data.

Apoptosis is one of the major modes in which DOX leads to cell death; therefore a well-established biomarker, cleaved caspase-3, was used as a biomarker for apoptosis. It was found that DL7076 alone led to no expression in cleaved caspase-3, but when DOX (2.5 μM) was administered to cells there was an abundance in the expression of cleaved caspase-3. This abundance of expression of cleaved caspase-3 is notably reduced in a concentration dependent manner when h9c2 cardiomyocytes are additionally pretreated with DL7076. The recovery in HO-1, additional to the reduction in cleaved caspase 3 expression, is not shown in the MDA-MB-231 (FIG. 41e) or BT549 (FIG. 41f), highlighting the benefits of tissue specificity Nrf2 activation. Taken together, DL7076 led to increased HO-1 expression and decreased cleaved caspase-3 expression in the presence of DOX, showing a tissue specific reversal of the DOX-induced apoptosis in H9c2 cardiomyocytes and not the other TNBC cells.

FIGS. 41g and 41h shows graphs of experimental data demonstrating HO-1 relative mRNA induction (FIG. 41g) and relative mRNA expression of HO-1 (FIG. 41g) for IPSC-derived cardiomyocytes treated with control, SFN (2.5 μM), or DL7076 (1 μM, 5 μM, 10 μM).

### DL7076 Selectively Reduces DOX-Induced Generation of ROS in H9c2 Cardiomyocytes

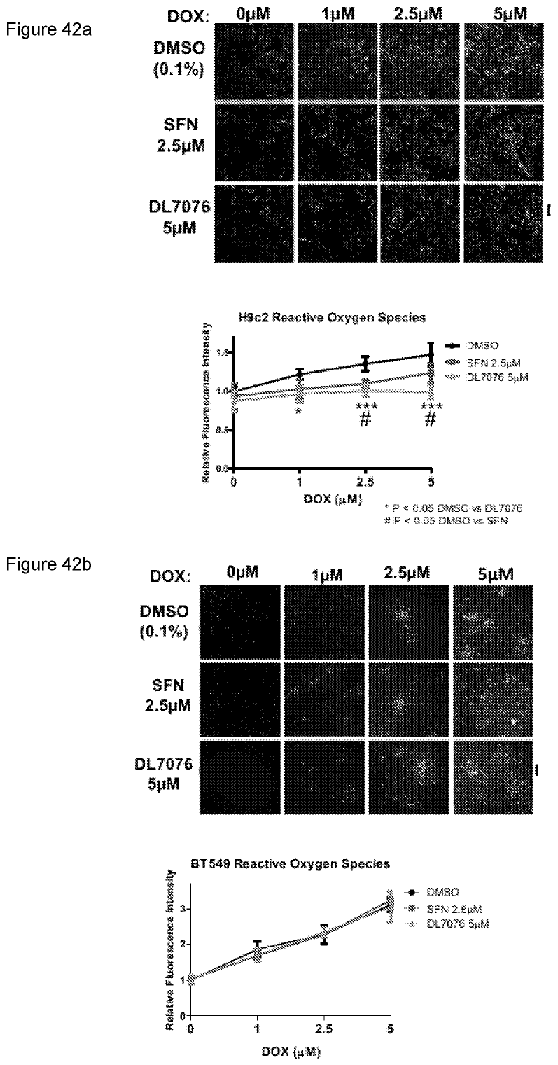

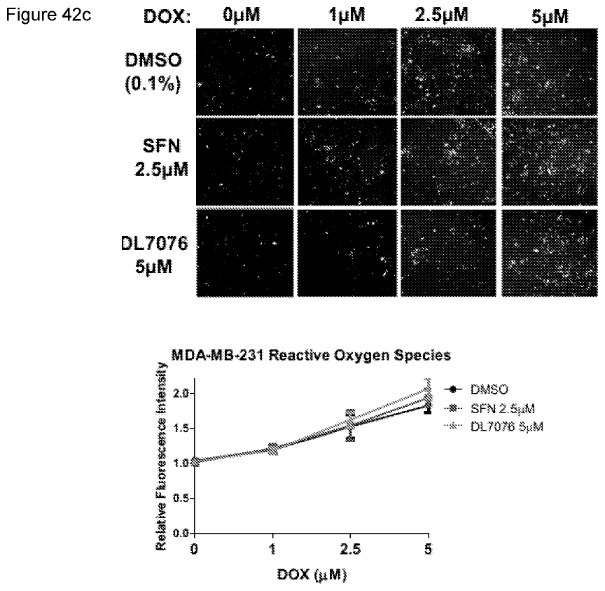

Oxidative stress and the modulation of mitochondrial ROS are important mechanisms through which DOX asserts its cardiotoxicity, leading to apoptosis. Oxidative stress in H9c2 cardiomyocytes induced by DOX and relieved by DL7076 cotreatment was thus investigated. As shown in FIG. 42a, DOX administration concentration dependently increases ROS, while a 2-hour pretreatment and 6-hour cotreatment of DL7076 with DOX lead to significantly reduced ROS, quantitated as a reduction in the fluorescence intensity. As described above, DL7076 has a tissue specific protection which is highlighted in FIG. 42b and FIG. 42c, showing significant ROS without reduction in the BT549 and MDA-MB-231 TNBC cell lines. Taken together, these results support further demonstrate that inclusion of DL7076 resulted in lesser ROS presence in H9c2 cardiomyocytes, but not in target TNBC cells.

### DL7076 Enhance CPA-Mediated Cytotoxicity and Reduced DOX-Induced H2AX Phosphorylation in an BT549/HPH/H9c2 Coculture System

To examine the effects of DL7076 on cardiotoxicity generated by DOX exposure and CPA-mediated target cell cytotoxicity, a novel multi-organ co-culture system was developed, as shown in FIG. 43a. This system incorporates H9c2 cardiomyocytes into the previous HPH/BT549 co-culture, allowing for cells to easily be identified from one another and further analyzed. Results from cell viability assays demonstrated that DL7076 treatment selectively enhanced CPA/DOX regimen cytotoxicity in BT549 cells (FIG. 43b). In the same co-culture system, the h9c2 cell cytotoxicity can be concurrently monitored, which demonstrated h9c2 cardiomyocyte viability can be protected during CPA/DOX treatment (FIG. 43c), reducing a major side-toxicity of CPA/DOX containing regimens. DNA double-stranded breaks (DSB) caused by chemotherapies lead to the phosphorylation of a variant of histone H2A, H2AX, which occurs rapidly, making H2AX a representative marker for DSB in cells. Through the utilization of immunochemical staining of H2AX, it was shown that the addition of DL7076 effectively enhanced the CPA/DOX-mediated DNA damage in the BT549 cells (FIG. 43d); however, in the H9c2 cardiomyocytes, a measured decrease in histone H2A phosphorylation was shown (FIG. 43e). Collectively, consistent with our earlier findings, these results confirm that the inclusion of DL7076 in CPA/DOX containing regimens led to significantly greater DNA damage in target TNBC cells and reduced DNA damage in off-target cardiomyocytes.

FIG. 44 shows a schematic illustration of DL7076 enhancement of CPA/DOX anticancer activity in target cells (BT549 and MDA-MB-231) and reduction of off-target toxicity in off target cells (H9c2).

### Discussion

As described herein, it was established that a dual activator selective for CAR and Nrf2 leads to enhanced CPA-mediated cytotoxicity in target TNBC cells and reduced cardiotoxicity in the off-target cardiomyocytes. Importantly, in the presence of DL7076, significantly lower concentrations CPA and DOX may be used, which led to comparable anticancer effects and a protective effect against DOX-induced cardiotoxicity.

As described herein, a novel multi-organ coculture system which incorporates HPH, BT549 TNBC cells, and H9c2 cells, allowing medium and nutrients to be shared was developed. This co-culture builds upon a previous co-culture system, wherein this new design enabled synchronous evaluation of CPA and DOX anticancer activity in BT549 cells and cardiotoxicity in H9c2 cells in the presence of functional human hepatocytes. The H9c2 cell line has been widely recognized as an acceptable in vitro cell model for DOX-induced cardiotoxicity.

Markedly, this study has shown, with the use of this novel coculture system, treatment with DL7076 in combination with CPA/DOX led to enhanced cytotoxicity in BT549, while reducing cytotoxicity in the H9c2 cells. While in vitro cell-based models cannot fully elucidate clinical benefit, these results indicate the potential that inclusion of a hCAR/Nrf2 dual activator may improve the therapeutic index of CPA/DOX regimens by lowering the required dose while achieving efficacy and lowered side toxicities in TNBC patients. Although this initial HPH/BT549/H9c2 coculture system represented promising anticancer activity in the BT549 cells and hindered cardiotoxicity in the H9c2 cells with the inclusion of DL7076 in the CPA/DOX containing regimen, this simplified in vitro model does not include other side toxicities that may contribute to the overall discontinuation of the regimen.

Collectively, the results show the overall improvement in cytotoxicity and cardiotoxicity of CPA and DOX containing regimens provided by selective activation of CAR and Nrf2 in TNBC cells. The enhanced formation of 4-OH-CPA, the rate-limiting metabolite, is mediated by the activation of CAR, and subsequent selective induction of CYP2B6 in human hepatocytes enables tissue specific cytotoxicity towards TNBC cells, while modulating Nrf2 alleviated cardiotoxicity in cardiomyocyte cells via HO-1 upregulation. This may benefit the readily clinically used CPA and DOX containing treatment regimens for TNBC. While considering that cell-based in vitro models have clear limitations and only represent a simplified view, the newly established multi-organ coculture system provides an effective cellular environment, allowing monitoring of hepatic metabolism, drug-drug interaction, antineoplastic activity, and off-target side effects of antineoplastic agents in multiple cell types, concomitantly.

### REFERENCES

1. U.S. Breast Cancer Statistics. BreastCancer.org. https://www.breastcancer.org/symptoms/understand_bc/statistics. Published 2019.
2. Triple Negative Breast Cancer Outlook: Survival Rates. Healthline.
3. Reis-filho J S, Ph D. Triple-Negative Breast Cancer. 2010.
4. Zeichner S B, Terawaki H, Gogineni K. A review of systemic treatment in metastatic triple-negative breast cancer. Breast Cancer Basic Clin Res. 2016. doi:10.4137/BCBCr.s32783.
5. Sharma P, Barlow W E, Godwin A K, et al. Impact of homologous recombination deficiency biomarkers on outcomes in patients with triplenegative breast cancer treated with adjuvant doxorubicin and cyclophosphamide (SWOG 59313). Ann Oncol. 2018. doi:10.1093/annonc/mdx821.
6. Myles A, Friedman O M, Rao S S, Colvin M. Identification of Aldophosphamide as a Metabolite of Cyclophosphamide in Vitro and in Vivo in Humans. Cancer Res. 1977.
7. Hedrich W D, Xiao J, Heyward S, et al. Activation of the Constitutive Androstane Receptor Increases the Therapeutic Index of CHOP in Lymphoma Treatment. Mol Cancer Ther. 2016. doi:10.1158/1535-7163.mct-15-0667.
8. Huang Z, Roy P, Waxman D J. Role of human liver microsomal CYP3A4 and CYP2B6 in catalyzing N-dechloroethylation of cyclophosphamide and ifosfamide. Biochem Pharmacol. 2000. doi:10.1016/S0006-2952(99)00410-4.
9. Code E L, Crespi C L, Penman B W, Gonzalez F J, Chang T K H, Waxman D J. Human cytochrome P4502B6: Interindividual hepatic expression, substrate specificity, and role in procarcinogen activation. Drug Metab Dispos. 1997.
10. Hedrich W, Xiao J, Heyward S, et al. No Title. Mol Cancer Ther. 2016; 15(3):392-401. doi:10.1158/1535-7163.MCT-15-0667.
11. Roy P, Yu L J, Crespi C L, Waxman D J. Development of a substrate-activity based approach to identify the major human liver P-450 catalysts of cyclophosphamide and ifosfamide activation based on cDNA-expressed activities and liver microsomal P-450 profiles. Drug Metab Dispos. 1999.
12. Yu L, Waxman D J. Role of cytochrome P450 in oxazaphosphorine metabolism: Deactivation via N-dechloroethylation and activation via 4-hydroxylation catalyzed by distinct subsets of rat liver cytochromes P450. Drug Metab Dispos. 1996.
13. Schwartz P S, Waxman D J. Cyclophosphamide induces caspase 9-dependent apoptosis in 9 L tumor cells. Mol Pharmacol. 2001. doi:10.1124/mol.60.6.1268
14. Hickman J A. Apoptosis induced by anticancer drugs. Cancer Metastasis Rev. 1992. doi:10.1007/BF00048059.
15. Wang D, Li L, Yang H, et al. The constitutive androstane receptor is a novel therapeutic target facilitating cyclophosphamide-based treatment of hematopoietic malignancies. Blood. 2013. doi:10.1182/blood-2012-06-436691.
16. Hedrich W D, Hassan H E, Wang H. Insights into CYP2B6-mediated drug-drug interactions. Acta Pharm Sin B. 2016; 6(5):413-425. doi:10.1016/j.apsb.2016.07.016.
17. Sueyoshi T, Kawamoto T, Zelko I, Honkakoski P, Negishi M. The repressed nuclear receptor CAR responds to phenobarbital in activating the human CYP2B6 gene. J Biol Chem. 1999. doi:10.1074/jbc.274.10.6043.
18. Mackowiak B, Hodge J, Stem S, Wang H. The roles of xenobiotic receptors: Beyond chemical disposition. Drug Metab Dispos. 2018; 46(9):1361-1371. doi:10.1124/dmd.118.081042.
19. Faucette S R, Sueyoshi T, Smith C M, Negishi M, Lecluyse E L, Wang H. Differential regulation of hepatic CYP2B6 and CYP3A4 genes by constitutive androstane receptor but not pregnane X receptor. J Pharmacol Exp Ther. 2006. doi:10.1124/jpet.105.098160.
20. Bai Y, Chen Q, Sun Y P, et al. Sulforaphane protection against the development of doxorubicin-induced chronic heart failure is associated with Nrf2 Upregulation. Cardiovasc Ther. 2017; 35(5):1-12. doi:10.1111/1755-5922.12277.
21. Chlebowski R T. Adriamycin (doxorubicin) cardiotoxicity: A review. West J Med. 1979.
22. Raber I, Asnani A. Cardioprotection in cancer therapy: Novel insights with anthracyclines. Cardiovasc Res. 2019. doi:10.1093/cvr/cvz023.
23. Liu Y, Asnani A, Zou L, et al. Visnagin protects against doxorubicin-induced cardiomyopathy through modulation of mitochondrial malate dehydrogenase. Sci Transl Med. 2014. doi:10.1126/scitranslmed.3010189.
24. Songbo M, Lang H, Xinyong C, Bin X, Ping Z, Liang S. Oxidative stress injury in doxorubicin-induced cardiotoxicity. Toxicol Lett. 2019. doi:10.1016/j.toxlet.2019.02.013.
25. Farias J G, Molina V M, Carrasco R A, et al. Antioxidant therapeutic strategies for cardiovascular conditions associated with oxidative stress. Nutrients. 2017. doi:10.3390/nu9090966.
26. Deidda M, Madonna R, Mango R, et al. Novel insights in pathophysiology of antiblastic drugs-induced cardiotoxicity and cardioprotection. J Cardiovasc Med. 2016. doi:10.2459/JCM.0000000000000373.
27. Cappetta D, De Angelis A, Sapio L, et al. Oxidative stress and cellular response to doxorubicin: A common factor in the complex milieu of anthracycline cardiotoxicity. Oxid Med Cell Longev. 2017. doi:10.1155/2017/1521020.
28. Renu K, V. G. A, Tirupathi T P, Arunachalam S. Molecular mechanism of doxorubicin-induced cardiomyopathy—An update. Eur J Pharmacol. 2018. doi:10.1016/j.ejphar.2017.10.043.
29. Singh P, Sharma R, McElhanon K, et al. Sulforaphane protects the heart from doxorubicin-induced toxicity. Free Radic Biol Med. 2015. doi:10.1016/j.freeradbiomed.2015.05.028.
30. Li B, Kim D S, Yadav R K, Kim H R, Chae H J. Sulforaphane prevents doxorubicin-induced oxidative stress and cell death in rat H9c2 cells. Int J Mol Med. 2015; 36(1):53-64. doi:10.3892/ijmm.2015.2199.
31. Childs A C, Phaneuf S L, Dirks A J, Phillips T, Leeuwenburgh C. Doxorubicin treatment in vivo causes cytochrome c release and cardiomyocyte apoptosis, as well as increased mitochondrial efficiency, superoxide dismutase activity, and Bcl-2:Bax ratio. Cancer Res. 2002.
32. Shan Q, Li X, Zheng M, et al. Protective effects of dimethyl itaconate in mice acute cardiotoxicity induced by doxorubicin. Biochem Biophys Res Commun. 2019; 517(3):538-544.
33. Menna P, Paz O, Chello M, Convino E, Salvatorelli E, Minotti G. No Title. Expert Opin Drug Saf. 2012; Suppl 1:S21-36. doi:10.1517/14740338.2011.589834.
34. Loboda A, Damulewicz M, Pyza E, Jozkowicz A, Dulak J. Role of Nrf2/HO-1 system in development, oxidative stress response and diseases: an evolutionarily conserved mechanism. Cell Mol Life Sci. 2016; 73(17):3221-3247. doi:10.1007/s00018-016-2223-0.
35. Dinkova-Kostova A T, Fahey J W, Kostov R V., Kensler T W. KEAP1 and done?Targeting the NRF2 pathway with sulforaphane. Trends Food Sci Technol. 2017; 69:257-269. doi:10.1016/j.tifs.2017.02.002.
36. Canning P, Sorrell F J, Bullock A N. Structural basis of Keap1 interactions with Nrf2. Free Radic Biol Med. 2015; 88(Part B):101-107. doi:10.1016/j.freeradbiomed.2015.05.034.
37. Mi-Kyoung Kwak and Thomas W. Kensler. Targeting NRF2 Signaling for Cancer Chemoprevention. Toxicol Appl Pharmacol. 2013; 244(1):66-76. doi:10.1016/j.taap.2009.08.028.
38. Faucette S R, Zhang T C, Moore R, et al. Relative activation of human pregnane X receptor versus constitutive androstane receptor defines distinct classes of CYP2B6 and CYP3A4 inducers. J Pharmacol Exp Ther. 2007. doi:10.1124/jpet.106.112136.
39. Hull T D, Boddu R, Guo L, et al. Heme oxygenase-1 regulates mitochondrial quality control in the heart. JCI Insight. 2016. doi:10.1172/jci.insight.85817.
40. Piantadosi C A, Carraway M S, Babiker A, Suliman H B. Heme oxygenase-1 regulates cardiac mitochondrial biogenesis via nrf2-mediated transcriptional control of nuclear respiratory factor-1. Circ Res. 2008. doi:10.1161/01.RES.0000338597.71702.ad.
41. Issan Y, Komowski R, Aravot D, et al. Heme oxygenase-1 induction improves cardiac function following myocardial ischemia by reducing oxidative stress. PLoS One. 2014. doi:10.1371/journal.pone.0092246.
42. Liang D, Li L, Lynch C, et al. Human constitutive androstane receptor agonist DL5016: A novel sensitizer for cyclophosphamide-based chemotherapies. Eur J Med Chem. 2019. doi:10.1016/j.ejmech.2019.06.031.
43. Kannan K, Jain S K. Oxidative stress and apoptosis. Pathophysiology. 2000. doi:10.1016/50928-4680(00)00053-5.
44. Kim S Y, Kim S J, Kim B J, et al. Doxorubicin-induced reactive oxygen species generation and intracellular Ca2+ increase are reciprocally modulated in rat cardiomyocytes. Exp Mol Med. 2006. doi:10.1038/emm.2006.63.
45. Tsang W P, Chau S P Y, Kong S K, Fung K P, Kwok T T. Reactive oxygen species mediate doxorubicin induced p53-independent apoptosis. Life Sci. 2003. doi:10.1016/S0024-3205(03)00566-6.
46. Kuo L J, Yang L X. ??-H2AX—A novel biomaker for DNA double-strand breaks. In Vivo (Brooklyn). 2008.
47. Giacchetti S, Porcher R, Lehmann-Che J, et al. Long-term survival of advanced triple-negative breast cancers with a dose-intense cyclophosphamide/anthracycline neoadjuvant regimen. Br J Cancer. 2014; 110(6):1413-1419. doi:10.1038/bjc.2014.81.
48. Wahba H A, El-Hadaad H A. Current approaches in treatment of triple-negative breast cancer. Cancer Biol Med. 2015. doi:10.7497/j.issn.2095-3941.2015.0030.
49. Bose C, Awasthi S, Sharma R, et al. Sulforaphane potentiates anticancer effects of doxorubicin and attenuates its cardiotoxicity in a breast cancer model. PLoS One. 2018; 13(3):1-22. doi:10.1371/journal.pone.0193918.
50. Das B N, Kim Y W, Keum Y S. Mechanisms of Nrf2/keap1-dependent phase II cytoprotective and detoxifying gene expression and potential cellular targets of chemopreventive isothiocyanates. Oxid Med Cell Longev. 2013; 2013. doi:10.1155/2013/839409.
51. Daher I N, Daigle T R, Bhatia N, Durand J B. The prevention of cardiovascular disease in cancer survivors. Texas Hear Inst J. 2012.
52. Turakhia S, Venkatakrishnan C D, Dunsmore K, et al. Doxorubicin-induced cardiotoxicity: Direct correlation of cardiac fibroblast and H9c2 cell survival and aconitase activity with heat shock protein 27. Am J Physiol—Hear Circ Physiol. 2007. doi:10.1152/ajpheart.00328.2007.
53. Singh P, Sharma R, Mcelhanon K, et al. Sulforaphane protects the heart from doxorubicin-induced toxicity HHS Public Access Graphical abstract. Free Radic Biol Med. 2015; 86:90-101. doi:10.1016/j.freeradbiomed.2015.05.028.
54. Teraoka K, Hirano M, Yamaguchi K, Yamashina A. Progressive cardiac dysfunction in adriamycin-induced cardiomyopathy rats. Eur J Heart Fail. 2000. doi:10.1016/S1388-9842(00)00111-2.
55. Scott J M, Khakoo A, MacKey J R, Haykowsky M J, Douglas P S, Jones L W.

Modulation of anthracycline-induced cardiotoxicity by aerobic exercise in breast cancer: Current evidence and underlying mechanisms. Circulation. 2011. doi:10.1161/CIRCULATIONAHA.111.021774.

1. A compound of formula (I), or a pharmaceutically acceptable salt, solvate, hydrate, cocrystal, or prodrug thereof: 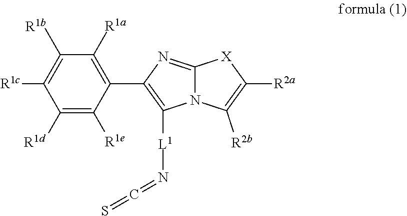
wherein in formula (I):
R^{1a}, R^{1b}, R^{1c}, R^{1d}, R^{1e}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl, and wherein R^{1a} and R^{1b}, R^{1b} and R^{1C}, R^{1c} and R^{1d}, R^{1d} and R^{1e}, and/or R^{2a} and R^{2b} are optionally joined together to form an optionally substituted aryl ring;
X is O or S;
L¹ is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

2. The compound of claim 1, or a pharmaceutically acceptable salt, solvate, hydrate, cocrystal, or prodrug thereof, wherein the compound has formula (1): 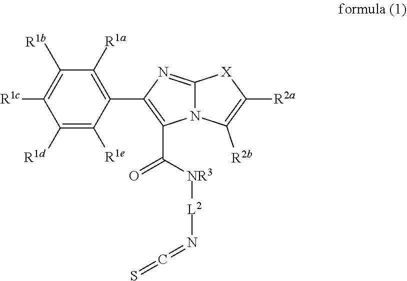
wherein in formula (1):
R³ is H or optionally substituted alkyl; and
L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

3. The compound of claim 1, wherein R^{1a}, R^{1b}, R^{1c}, R^{1d}, and R^{1e} are each independently selected from H, halo, and trifluoromethyl.

4. The compound of claim 1 or 2, wherein Rio is selected from F, Cl, and trifluoromethyl.

5. The compound of claim 1 or 2, wherein R^{1a}, R^{1b}, R^{1c}, R^{1d}, and R^{1e} are each H.

6. The compound of claim 1 or 2, wherein R^{1b} and R^{1c} are joined together to form an optionally substituted aryl ring.

7. The compound of claim 1, or a pharmaceutically acceptable salt, solvate, hydrate, cocrystal, or prodrug thereof, wherein the compound has formula (2): 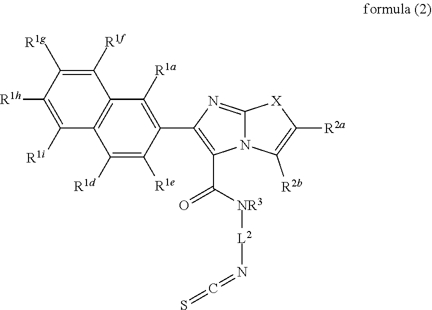
wherein in formula (2):
R^{1h}, R^{1g}, R^{1h}, and R¹ⁱ are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heterocyclyl;
R³ is H or optionally substituted alkyl; and
L² is a linker selected from optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, and optionally substituted heteroaryl, and combinations thereof, or L is joined to R³ to form a ring.

8. The compound of claim 7, wherein R^{1a}, R^{1d}, R^{1e}, R^{1f}, R^{1g}, R^{1h}, and R¹ⁱ are each H.

9. The compound of any one of claims 1-8, wherein R^{2a} and R^{2b} are each H.

10. The compound of any one of claims 1-9, wherein R³ is H.

11. The compound of any one of claims 1-10, wherein X is O.

12. The compound of any one of claims 1-10, wherein X is S.

13. The compound of any one of claims 2-12, wherein L² is selected from optionally substituted C₂-C₄ alkyl, optionally substituted cycloalkyl, and optionally substituted heterocyclyl.

14. The compound of any one of claims 2-12, wherein L² is selected from,

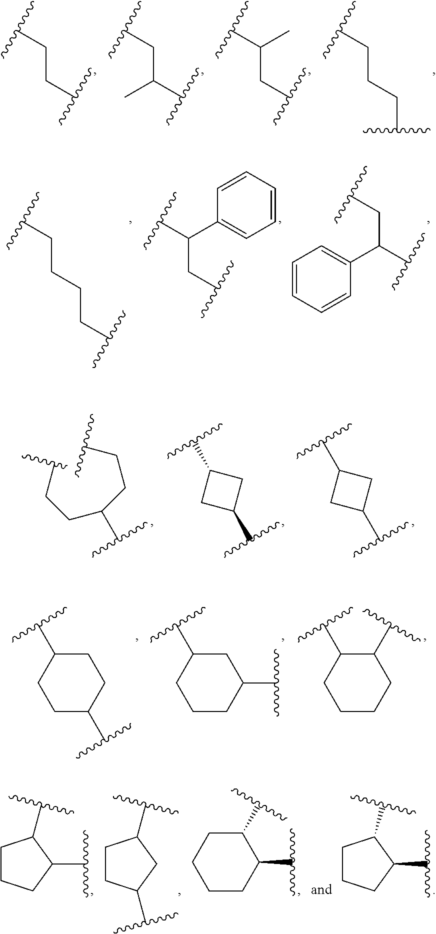

15. The compound of claim 2 or 7, wherein the compound of formula (1) or formula (2) is a compound of formula (11) or (12), or a pharmaceutically acceptable salt, solvate, hydrate, cocrystal, or prodrug thereof:

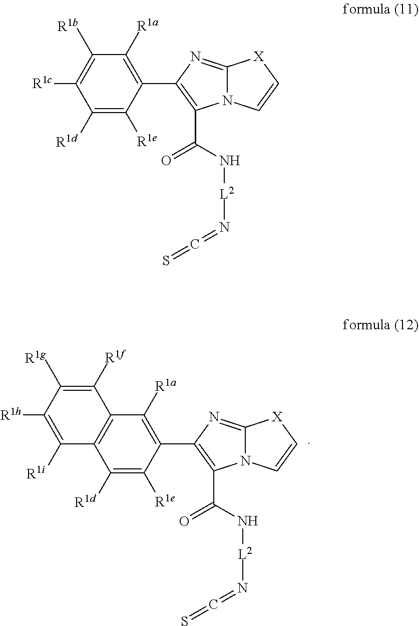

16. The compound of claim 2 or 7, wherein the compound of formula (I) is a compound of formula (21) or (22), or a pharmaceutically acceptable salt, solvate, hydrate, cocrystal, or prodrug thereof:

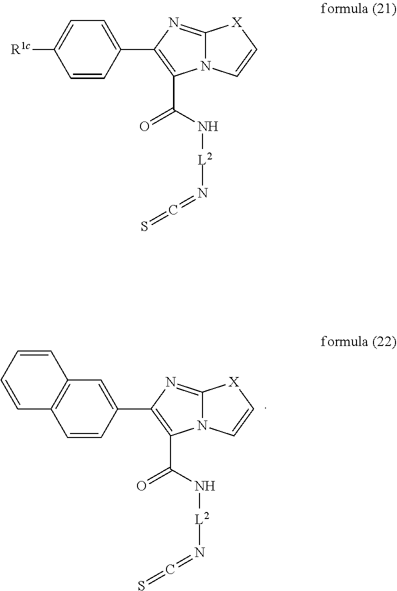

17. The compound of claim 2 or claim 2, wherein the compound of formula (1) or (2) is a compound of formula (31) or (32), or a pharmaceutically acceptable salt, solvate, hydrate, cocrystal, or prodrug thereof:

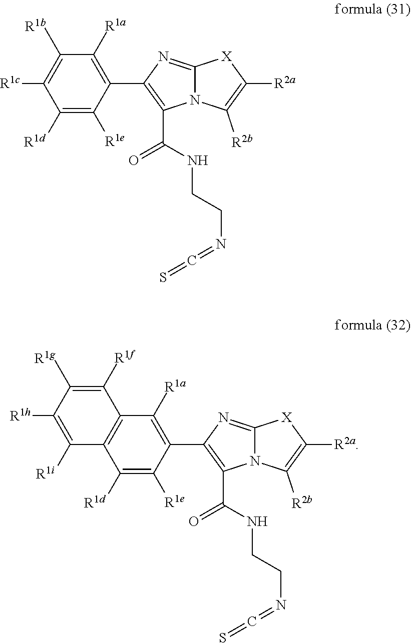

18. The compound of claims 15-17, wherein R^{1c} is selected from F, Cl, and trifluoromethyl.

19. The compound of claim 2, wherein the compound is of any one of formulas 1001 to 1096, or a pharmaceutically acceptable salt, solvate, hydrate, cocrystal, or prodrug thereof:

| | | | formula (21) |
|---|---|---|---|
| 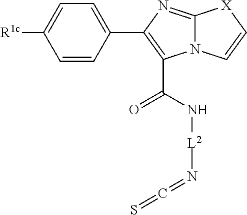 | | | |
| Compound | | | |
| # | R^{1c} | X | L² |
| 1001 | H | O | 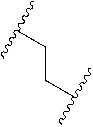 [SMILES: *CC*] |
| 1002 | —F | O | 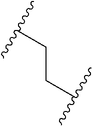 [SMILES: *CC*] |
| 1003 | —Cl | O | 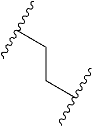 [SMILES: *CC*] |
| 1004 | —CF₃ | O | 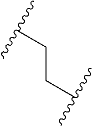 [SMILES: *CC*] |
| 1005 | H | O | 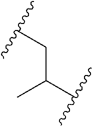 [SMILES: *CC(*)C] |
| 1006 | —F | O | 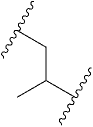 [SMILES: *CC(*)C] |
| 1007 | —Cl | O | 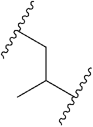 [SMILES: *CC(*)C] |
| 1008 | —CFs | O | 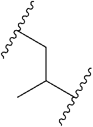 [SMILES: *CC(*)C] |
| 1009 | H | O | 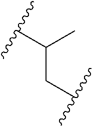 [SMILES: *CC(*)C] |
| 1010 | —F | O | 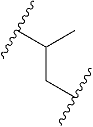 [SMILES: *CC(*)C] |
| 1011 | —Cl | O | 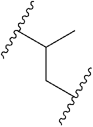 [SMILES: *CC(*)C] |
| 1012 | —CFs | O | 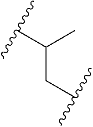 [SMILES: *CC(*)C] |
| 1013 | H | O | 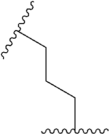 [SMILES: *CCC*] |
| 1014 | —F | O | 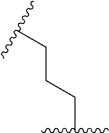 [SMILES: *CCC*] |
| 1015 | —Cl | O | 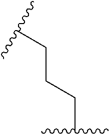 [SMILES: *CCC*] |
| 1016 | —CF₃ | O | 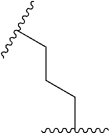 [SMILES: *CCC*] |
| 1017 | H | O | 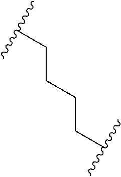 [SMILES: *CCCC*] |
| 1018 | —F | O | 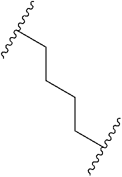 [SMILES: *CCCC*] |
| 1019 | —Cl | O | 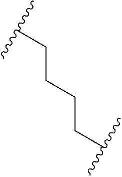 [SMILES: *CCCC*] |
| 1020 | —CFs | O | 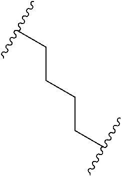 [SMILES: *CCCC*] |
| 1021 | H | O | 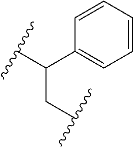 [SMILES: *CC(*)c1ccccc1] |
| 1022 | —F | O | 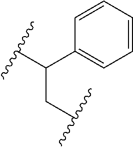 [SMILES: *CC(*)c1ccccc1] |
| 1023 | —Cl | O | 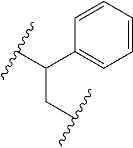 [SMILES: *CC(*)c1ccccc1] |
| 1024 | —CFs | O | 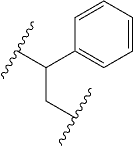 [SMILES: *CC(*)c1ccccc1] |
| 1025 | H | O | 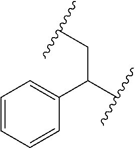 [SMILES: *CC(*)c1ccccc1] |
| 1026 | —F | O | 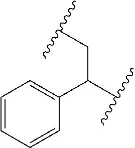 [SMILES: *CC(*)c1ccccc1] |
| 1027 | —Cl | O | 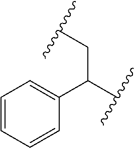 [SMILES: *CC(*)c1ccccc1] |
| 1028 | —CF₃ | O | 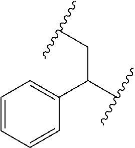 [SMILES: *CC(*)c1ccccc1] |
| 1029 | H | O | 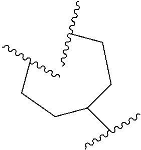 [SMILES: *CCC(*)CC*] |
| 1030 | —F | O | 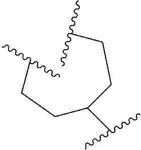 [SMILES: *CCC(*)CC*] |
| 1031 | —Cl | O | 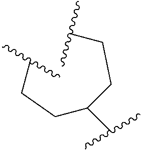 [SMILES: *CCC(*)CC*] |
| 1032 | —CFs | O | 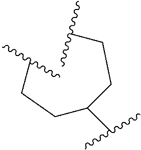 [SMILES: *CCC(*)CC*] |
| 1033 | H | O | 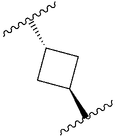 [SMILES: *[C@H]1C[C@H](*)C1] |
| 1034 | —F | O | 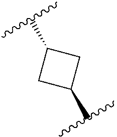 [SMILES: *[C@H]1C[C@H](*)C1] |
| 1035 | —Cl | O | 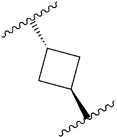 [SMILES: *[C@H]1C[C@H](*)C1] |
| 1036 | —CFs | O | 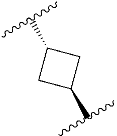 [SMILES: *[C@H]1C[C@H](*)C1] |
| 1037 | H | O | 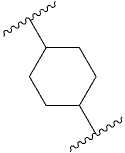 [SMILES: *C1CCC(*)CC1] |
| 1038 | —F | O | 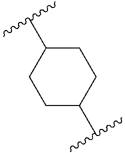 [SMILES: *C1CCC(*)CC1] |
| 1039 | —Cl | O | 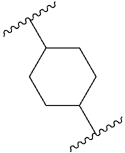 [SMILES: *C1CCC(*)CC1] |
| 1040 | —CF₃ | O | 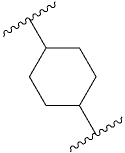 [SMILES: *C1CCC(*)CC1] |
| 1041 | H | O | 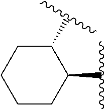 [SMILES: *[C@H]1CCCC[C@@H]1*] |
| 1042 | —F | O | 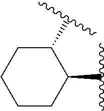 [SMILES: *[C@H]1CCCC[C@@H]1*] |
| 1043 | —Cl | O | 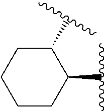 [SMILES: *[C@H]1CCCC[C@@H]1*] |
| 1044 | —CFs | O | 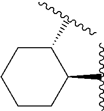 [SMILES: *[C@H]1CCCC[C@@H]1*] |
| 1045 | H | O | 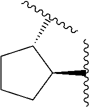 [SMILES: *[C@H]1CCC[C@@H]1*] |
| 1046 | —F | O | 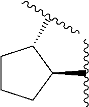 [SMILES: *[C@H]1CCC[C@@H]1*] |
| 1047 | —Cl | O | 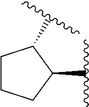 [SMILES: *[C@H]1CCC[C@@H]1*] |
| 1048 | —CFs | O | 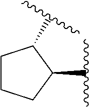 [SMILES: *[C@H]1CCC[C@@H]1*] |
| 1049 | H | S | 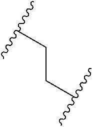 [SMILES: *CC*] |
| 1050 | —F | S | 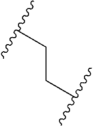 [SMILES: *CC*] |
| 1051 | —Cl | S | 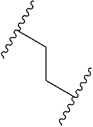 [SMILES: *CC*] |
| 1052 | —CF₃ | S | 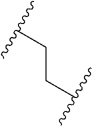 [SMILES: *CC*] |
| 1053 | H | S | 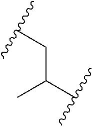 [SMILES: *CC(*)C] |
| 1054 | —F | S | 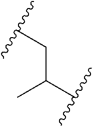 [SMILES: *CC(*)C] |
| 1055 | —Cl | S | 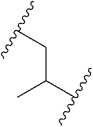 [SMILES: *CC(*)C] |
| 1056 | —CFs | S | 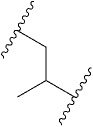 [SMILES: *CC(*)C] |
| 1057 | H | S | 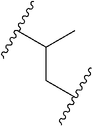 [SMILES: *CC(*)C] |
| 1058 | —F | S | 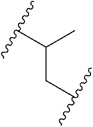 [SMILES: *CC(*)C] |
| 1059 | —Cl | S | 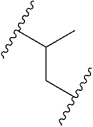 [SMILES: *CC(*)C] |
| 1060 | —CFs | S | 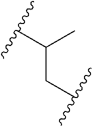 [SMILES: *CC(*)C] |
| 1061 | H | S | 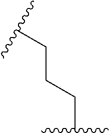 [SMILES: *CCC*] |
| 1062 | —F | S | 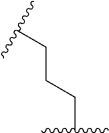 [SMILES: *CCC*] |
| 1063 | —Cl | S | 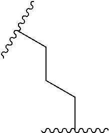 [SMILES: *CCC*] |
| 1064 | —CF₃ | S | 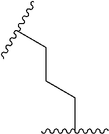 [SMILES: *CCC*] |
| 1065 | H | S | 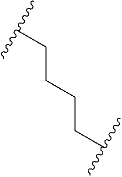 [SMILES: *CCCC*] |
| 1066 | —F | S | 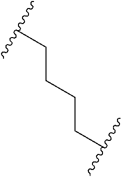 [SMILES: *CCCC*] |
| 1067 | —Cl | S | 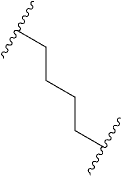 [SMILES: *CCCC*] |
| 1068 | —CFs | S | 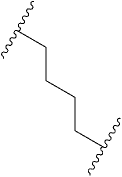 [SMILES: *CCCC*] |
| 1069 | H | S | 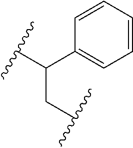 [SMILES: *CC(*)c1ccccc1] |
| 1070 | —F | S | 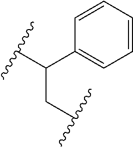 [SMILES: *CC(*)c1ccccc1] |
| 1071 | —Cl | S | 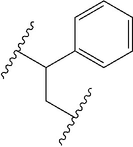 [SMILES: *CC(*)c1ccccc1] |
| 1072 | —CFs | S | 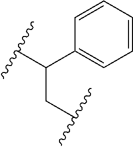 [SMILES: *CC(*)c1ccccc1] |
| 1073 | H | S | 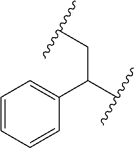 [SMILES: *CC(*)c1ccccc1] |
| 1074 | —F | S | 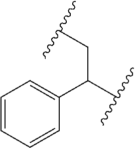 [SMILES: *CC(*)c1ccccc1] |
| 1075 | —Cl | S | 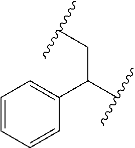 [SMILES: *CC(*)c1ccccc1] |
| 1076 | —CF₃ | S | 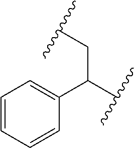 [SMILES: *CC(*)c1ccccc1] |
| 1077 | H | S | 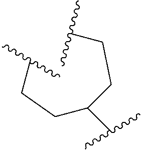 [SMILES: *CCC(*)CC*] |
| 1078 | —F | S | 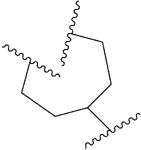 [SMILES: *CCC(*)CC*] |
| 1079 | —Cl | S | 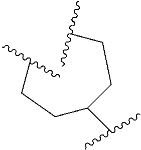 [SMILES: *CCC(*)CC*] |
| 1080 | —CFs | S | 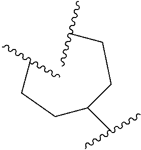 [SMILES: *CCC(*)CC*] |
| 1081 | H | S | 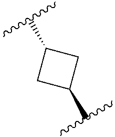 [SMILES: *[C@H]1C[C@H](*)C1] |
| 1082 | —F | S | 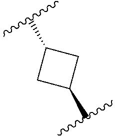 [SMILES: *[C@H]1C[C@H](*)C1] |
| 1083 | —Cl | S | 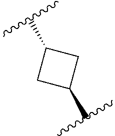 [SMILES: *[C@H]1C[C@H](*)C1] |
| 1084 | —CF3 | S | 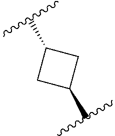 [SMILES: *[C@H]1C[C@H](*)C1] |
| 1085 | H | S | 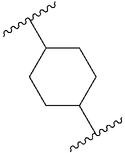 [SMILES: *C1CCC(*)CC1] |
| 1086 | —F | S | 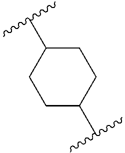 [SMILES: *C1CCC(*)CC1] |
| 1087 | —Cl | S | 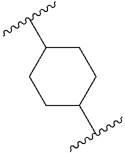 [SMILES: *C1CCC(*)CC1] |
| 1088 | —CF₃ | S | 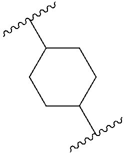 [SMILES: *C1CCC(*)CC1] |
| 1089 | H | S | 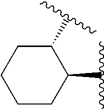 [SMILES: *[C@H]1CCCC[C@@H]1*] |
| 1090 | —F | S | 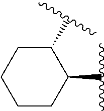 [SMILES: *[C@H]1CCCC[C@@H]1*] |
| 1091 | —Cl | S | 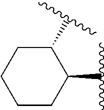 [SMILES: *[C@H]1CCCC[C@@H]1*] |
| 1092 | —CFs | S | 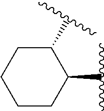 [SMILES: *[C@H]1CCCC[C@@H]1*] |
| 1093 | H | S | 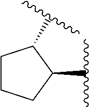 [SMILES: *[C@H]1CCC[C@@H]1*] |
| 1094 | —F | S | 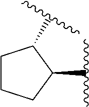 [SMILES: *[C@H]1CCC[C@@H]1*] |
| 1095 | —Cl | S | 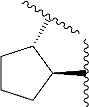 [SMILES: *[C@H]1CCC[C@@H]1*] |
| 1096 | —CFs | S | 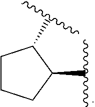 [SMILES: *[C@H]1CCC[C@@H]1*] |

19. The compound of claim 2, wherein the compound is of any one of formulas 2001 to 2024, or a pharmaceutically acceptable salt, solvate, hydrate, cocrystal, or prodrug thereof:

| | | formula (22) |
|---|---|---|
| 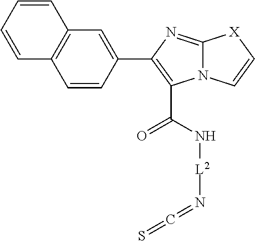 | | |
| Compound | | |
| # | X | L² |
| 2001 | O | 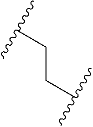 [SMILES: *CC*] |
| 2002 | O | 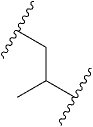 [SMILES: *CC(*)C] |
| 2003 | O | 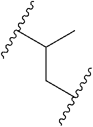 [SMILES: *CC(*)C] |
| 2004 | O | 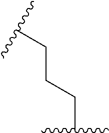 [SMILES: *CCC*] |
| 2005 | O | |
| 2006 | O | 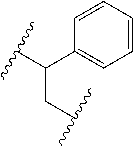 [SMILES: *CC(*)c1ccccc1] |
| 2007 | O | 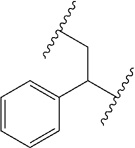 [SMILES: *CC(*)c1ccccc1] |
| 2008 | O | 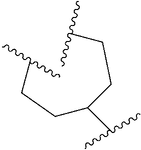 [SMILES: *CCC(*)CC*] |
| 2009 | O | 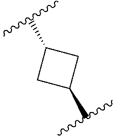 [SMILES: *[C@H]1C[C@H](*)C1] |
| 2010 | O | 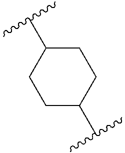 [SMILES: *C1CCC(*)CC1] |
| 2011 | O | 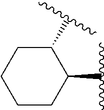 [SMILES: *[C@H]1CCCC[C@@H]1*] |
| 2012 | O | 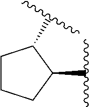 [SMILES: *[C@H]1CCC[C@@H]1*] |
| 2013 | S | 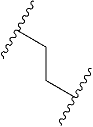 [SMILES: *CC*] |
| 2014 | S | 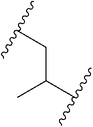 [SMILES: *CC(*)C] |
| 2015 | S | 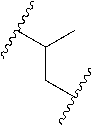 [SMILES: *CC(*)C] |
| 2016 | S | 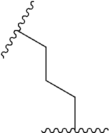 [SMILES: *CCC*] |
| 2017 | S | 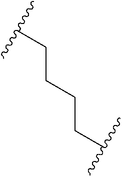 [SMILES: *CCCC*] |
| 2018 | S | 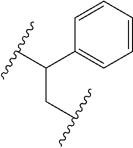 [SMILES: *CC(*)c1ccccc1] |
| 2019 | S | 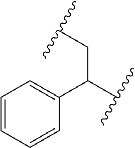 [SMILES: *CC(*)c1ccccc1] |
| 2020 | S | 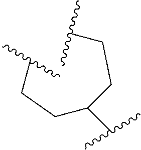 [SMILES: *CCC(*)CC*] |
| 2021 | S | 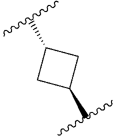 [SMILES: *[C@H]1C[C@H](*)C1] |
| 2022 | S | 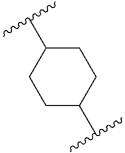 [SMILES: *C1CCC(*)CC1] |
| 2023 | S | 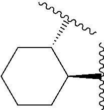 [SMILES: *[C@H]1CCCC[C@@H]1*] |
| 2024 | S | 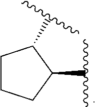 [SMILES: *[C@H]1CCC[C@@H]1*] |

21. The compound of claim 1, wherein the compound is selected from:

| Compound # | Structure |
|---|---|
| 2013 (DL7076) | 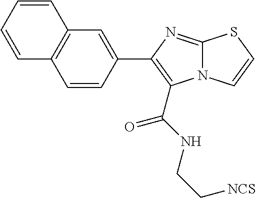 |
| 2001 (DL7009) | 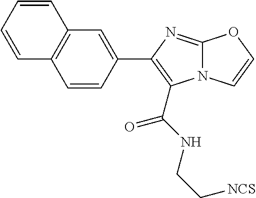 |
| 2002 (DL7092) | 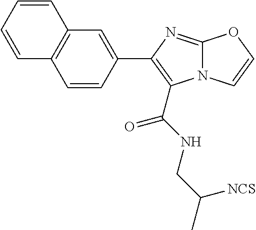 |
| 2003 (DL7091) | 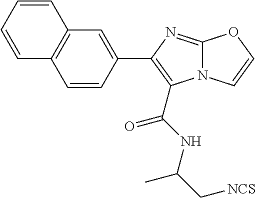 |
| 2007 (DL70552) | 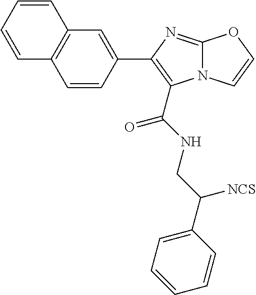 |
| 2006 (DL70562) | 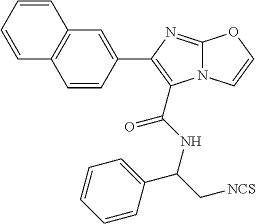 |
| 2008 (DL7077) | 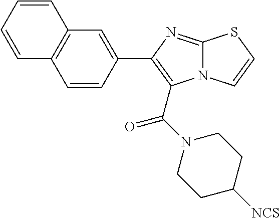 |
| 2009 (DL7102) | 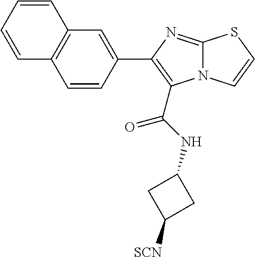 |
| 2010 (DL7086) | 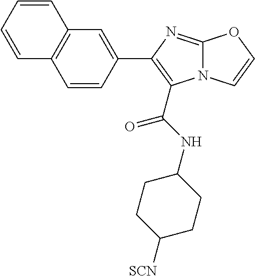 |
| 2011 (DL7096) | 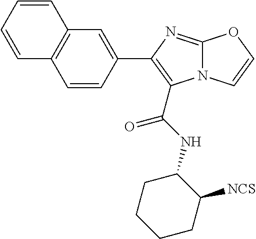 |
| 2012 (DL7097) | 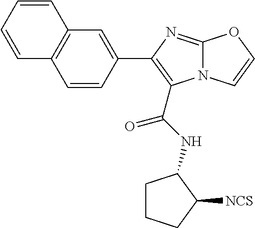 |
| 2004 (DL7087) | 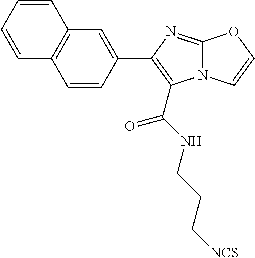 |
| 2005 (DL7101) | 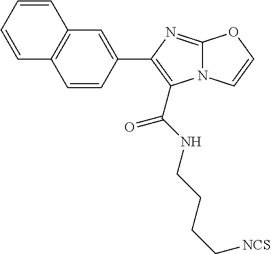 |
| 1049 (DL7134) | 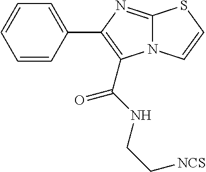 |
| 1050 (DL7135) | 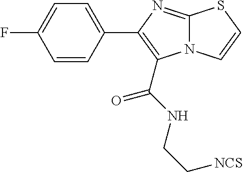 |
| 1051 (DL7128) | 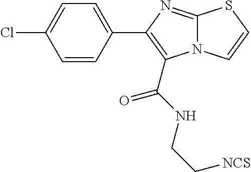 |
| 1052 (DL7127) | 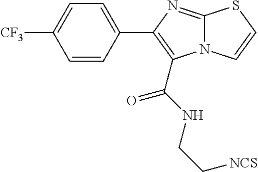 |
| 2015 (DL7139) | 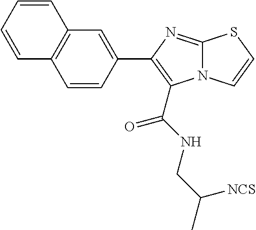 |
| 2014 (DL7140) | 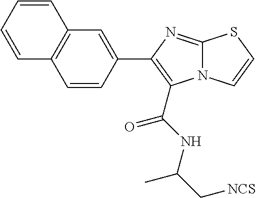 |

or a pharmaceutically acceptable salt, solvate, hydrate, cocrystal, or prodrug thereof.

22. The compound of claim 1, wherein the compound is 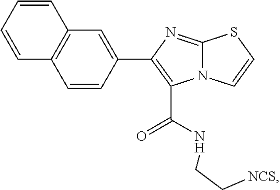
or a pharmaceutically acceptable salt, solvate, hydrate, cocrystal, or prodrug thereof.

23. The compound of any one of claims 1-22, wherein the compound is a hCAR activator.

24. The compound of any one of claims 1-22, wherein the compound is a Nrf2 activator.

25. The compound of any one of claims 1-21, wherein the compound is a dual hCAR and Nrf2 activator.

26. A pharmaceutical composition comprising a compound of any one of claims 1-25, or a pharmaceutically acceptable salt, solvate, hydrate, cocrystal, or prodrug thereof, and a physiologically compatible carrier medium.

27. A method of treating a disease alleviated by activating hCAR in a patient in need thereof, the method comprising administering to the patient a therapeutically effective amount of a compound of any one of claims 1-25, or a pharmaceutically acceptable salt, solvate, hydrate, cocrystal, or prodrug thereof.

28. A method of treating a disease alleviated by activating Nrf2 in a patient in need thereof, the method comprising administering to the patient a therapeutically effective amount of a compound of any one of claims 1-25, or a pharmaceutically acceptable salt, solvate, hydrate, cocrystal, or prodrug thereof.

29. A method of treating a disease alleviated by activating hCAR and Nrf2 in a patient in need thereof, comprising administering to the patient a therapeutically effective amount of a compound of any one of claims 1-25, or a pharmaceutically acceptable salt, solvate, hydrate, cocrystal, or prodrug thereof.

30. The method of any one of claims 27-29, wherein CYP2B6 is selectively induced over CYP3A4.

31. The method of any one of claims 27-30, the method further comprising administering to the patient a therapeutically effective amount of cyclophosphamide (CPA) and doxorubicin (DOX), or a pharmaceutically acceptable salt, solvate, hydrate, cocrystal, or prodrug thereof.

32. The method of claim 31, wherein CPA and DOX is administered as part of the CHOP regimen (CPA, doxorubicin, vincristine, and prednisone).

33. The method of any one of claims 31 or 32, wherein co-administration of a compound of any one of claims 1-25, or a pharmaceutically acceptable salt, solvate, hydrate, cocrystal, or prodrug thereof, CPA, and DOX, promotes the formation of therapeutically active CPA metabolite 4-OH-CPA and decreased cleaved caspase-3 expression.

34. The method of any one of claims 27-33, wherein the compound, or pharmaceutically acceptable salt, solvate, hydrate, cocrystal, or prodrug thereof, is administered in a dosage unit form.

35. The method of claim 35, wherein the dosage unit form comprises a physiologically compatible carrier medium.

36. The method of any one of claims 27-35, wherein the disease is cancer.

37. The method of claim 36, wherein the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

38. The method of claim 36, wherein the cancer is a triple negative breast cancer (TNBC).


SEQUENCE LISTING (8 sequences)

SEQ ID NO: 1 - DNA, 20 bp, artificial sequence
  Chemically synthesized
agacgccttcaatcctgacc

SEQ ID NO: 2 - DNA, 21 bp, artificial sequence
  Chemically synthesized
ccttcaccaagacaaatccgc

SEQ ID NO: 3 - DNA, 21 bp, artificial sequence
  Chemically synthesized
gtggggcttttatgatggtca

SEQ ID NO: 4 - DNA, 23 bp, artificial sequence
  Chemically synthesized
gcctcagatttctcaccaacaca

SEQ ID NO: 5 - DNA, 23 bp, artificial sequence
  Chemically synthesized
gggtgatagaagaggccaagact

SEQ ID NO: 6 - DNA, 21 bp, artificial sequence
  Chemically synthesized
agctcctgcaactcctcaaag

SEQ ID NO: 7 - DNA, 21 bp, artificial sequence
  Chemically synthesized
cccatcaccatcttccaggag

SEQ ID NO: 8 - DNA, 21 bp, artificial sequence
  Chemically synthesized
gttgtcatggatgaccttggc